(19) United States
(12) Patent Application Publication
(10) Pub. No.: US 2016/0058584 A1
(43) Pub. Date: Mar. 3, 2016

(54) PROSTHETIC SOCKET WITH AN ADJUSTABLE HEIGHT ISCHIAL SEAT

(71) Applicant: LIM INNOVATIONS, INC., San Francisco, CA (US)

(72) Inventors: Juan Jacobo Cespedes, San Francisco, CA (US); Garrett Ray Hurley, San Francisco, CA (US); Jesse Robert Williams, San Francisco, CA (US)

(21) Appl. No.: 14/844,462

(22) Filed: Sep. 3, 2015

Related U.S. Application Data
(63) Continuation-in-part of application No. 14/659,433, filed on Mar. 16, 2015.
(60) Provisional application No. 62/045,433, filed on Sep. 3, 2014.
(60) Provisional application No. 62/128,218, filed on Mar. 4, 2015.
(60) Provisional application No. 62/163,717, filed on May 19, 2015.
(60) Provisional application No. 62/161,132, filed on May 13, 2015.

(51) Int. Cl. A61F 2/80; B29C 67/00; G05B 19/4099; A61F 2/50; A61F 2/76

(52) U.S. Cl. CPC A61F 2/80; A61F 2/5046; A61F 2/76; G05B 19/4099; B29C 67/0088; A61F 2002/5024; A61F 2002/505; A61F 2002/5083; A61F 2002/608

(57) ABSTRACT

A modular prosthetic socket for a residual limb of a lower extremity of a patient may include an adjustable height ischial seat for facilitating the distribution of body weight away from the distal end of the residual limb and channeling the weight preferentially through the ischial tuberosity. In one aspect, the modular prosthetic socket may include a base plate, multiple longitudinal struts, and an ischial seat pad adjustably coupled with the proximal end of a medial strut, such that the ischial seat pad is vertically adjustable relative to the medial strut to adjust a total length of the medial strut measured from a proximal end of the ischial seat pad to the distal end of the medial strut. The ischial seat pad is configured to engage an ischium of the patient when the prosthetic socket is worn by the patient.

### CROSS-REFERENCE TO RELATED APPLICATIONS

This patent application claims the priority and benefit of U.S. Provisional Patent Applications: 62/045,433, filed Sep. 3, 2014, entitled “Modular Prosthetic Socket: Softgood Arrangements, Hardware, and a Flexible Inner Liner”; 62/128,218, filed Mar. 4, 2015, entitled “Modular Prosthetic Socket”; Ser. No. 62/163,717, filed May 19, 2015, entitled “Prosthetic Socket Distal Cup with a Strap Lanyard Suspension Mechanism and a Variable Elastic Modulus Cushion”; and 62/161,132, filed May 13, 2015, entitled “Prosthetic Socket that is Sensor Enabled to Provide Data for Clinical Use and Mechanical Adjustments.” These referenced provisional patent applications are hereby incorporated by reference in their entirety into the present patent application.

The present application is a continuation-in-part of U.S. patent application Ser. No. 14/659,433, entitled “Modular Prosthetic Sockets and Methods for Making Same,” filed Mar. 16, 2015.

The present application is related to U.S. patent application Ser. No. 13/675,761, entitled “Modular Prosthetic Sockets and Methods for Making Same,” filed Nov. 13, 2012; Ser. No. 14/213,788, entitled “Modular Prosthetic Sockets and Methods for Making and Using Same,” filed Mar. 14, 2014; and 62/007,742, entitled “Apparatus and Method for Transferring a Digital Profile of a Residual Limb to a Prosthetic Socket Strut,” filed Jun. 4, 2014. The above-referenced patent applications are hereby incorporated by reference in their entirety into the present patent application.

### INCORPORATION BY REFERENCE

All publications and patent applications identified in this specification are herein incorporated by reference to the same extent as if each such individual publication or patent application were specifically and individually indicated to be so incorporated by reference.

### TECHNICAL FIELD

The invention relates to medical devices and methods. More specifically, the invention relates to components of a modular prosthetic socket system, and to embodiments of an adjustable height ischial seat assembly applicable to prosthetic sockets.

### BACKGROUND

Prosthetic limbs for the upper and lower extremities typically include a residual limb socket, an alignment system, and a distal prosthetic component, such as a knee, foot, arm, or hand. For any prosthetic limb, the prosthetic socket is the portion of the prosthesis that fits on and grasps the residual limb and functionally connects the residual limb to more distal prosthetic components. If the prosthetic socket does not fit properly, indeed, if it does not fit extremely well, and if it cannot be adjusted easily by the patient, the function of distal components of the prosthetic can be severely compromised, and the patient may reject the prosthesis or use it only under duress.

Currently available methods for designing and making a prosthetic socket are very patient-specific, labor intensive and technically demanding. In a traditional and widespread “cottage industry” approach, the process begins by a prosthetist evaluating a patient's condition and needs and taking measurements of the patient's residual limb. Typically, the prosthetist then casts a negative mold of the residual limb with casting tape. This negative mold is filled with Plaster of Paris and allowed to harden. The negative cast is then peeled off to reveal the formed positive mold. The prosthetist may then modify the positive mold in an effort to create a form that best supports the creation of a limb socket that distributes pressure optimally on the residual limb when the socket is worn. The prosthetic socket is then built up by an iterative laminating of layers of polymer material over the positive mold. Finally, the positive mold is broken and removed from within the formed socket. The socket can then be cut or further modified to fit the residual limb of the patient.

When fabrication of the socket is complete, it is typically tested on the patient for fit and for the patient's sense of how it feels and works. Although a few minor modifications of the socket are possible at this stage, the scope of these possible modifications is limited. Accordingly, it is common practice to make a number of “check sockets” or “diagnostic sockets,” from which the best option is chosen as the final product for the patient.

Various aspects of this conventional prosthetic fabrication process are not ideal. The central role of physical molds in the fitting process and the transfer of size and shape information from the residual limb to the final prosthetic socket are limiting technological factors. The fabricating process itself can take weeks or even a month or more. And although the finished prosthetic socket product may often be quite satisfactory when produced by skilled prosthetists, it is still substantially fixed in form and cannot be easily modified, if at all. The residual limb itself, however, is not fixed in form. In fact, the residual limb often changes shape and condition radically, both in the short term and the long term.

Even if a prosthetic socket seems to fit perfectly in a prosthetist's office, the socket may rub or place pressure on the patient's residual limb when the limb is used repeatedly over days and weeks. Additionally, patients often lose or gain weight rather quickly as a result of their amputations or other complications of their life, thus causing the residual limb to grow or shrink. Similarly, as patients use their residual limbs with their prosthetic devices, they can build muscle and/or portions of the residual limb may change shape due to stresses placed on the residual limb during use. Finally, as the patient ages, the residual limb will continue to change, in response to continued use and environmental conditions. Using currently available techniques for making prosthetic sockets, anytime a significant change is needed in a socket for a patient, the only solution is to start the process again from step one and make a brand new socket.

Although improvements in prosthetic socket technology have been made, currently available prosthetic sockets still remain substantially fixed in shape and circumferential dimensions, particularly at in their distal portion. Additionally, the manufacturing process for prosthetic sockets continues to be labor intensive, time consuming, and technically demanding. Due to all the shortcomings of conventional prosthetic sockets and their manufacture, it would be desirable to have new prosthetic socket devices and systems, as well as new methods for making them. Ideally, such prosthetic sockets and manufacturing methods would lend themselves to modern manufacturing techniques and would help provide improved quality control and ability to scale production. Also welcome would be prosthetic sockets that could be manufactured at large scale, while still being highly customizable for each individual patient. It would also be advantageous to have a method of prosthetic socket manufacture that significantly shortened the average time required to deliver a finished, individualized prosthetic socket to a patient, compared to current techniques. Finally, it would be ideal to have prosthetic sockets that could be easily adjusted, to handle changes in residual limb size and shape over time, including both short and long term changes. At least some of these objectives will be met by the embodiments described herein.

### BRIEF SUMMARY

In one aspect, embodiments of the invention are directed to a prosthetic socket for a residual limb of a lower extremity of a patient. These prosthetic socket embodiments include a base plate having multiple peripherally disposed strut connecting sites. These embodiments further include multiple longitudinal struts, each strut having a thermoplastic-fiber composite material, a proximal end and a distal end, wherein the distal end of each strut is connected to peripherally disposed strut connecting sites of the base plate, and wherein one of the multiple struts is a medial strut that occupies a medial position with respect to the residual limb when the prosthetic socket is being worn by the patient. These embodiments further include an ischial seat pad adjustably coupled with the proximal end of the medial strut such that the ischial seat pad is vertically adjustable relative to the medial strut to adjust a total length of the medial strut measured from a proximal end of the ischial seat pad to the distal end of the medial strut, wherein the ischial seat pad is configured to engage an ischium of the patient when the prosthetic socket is worn by the patient.

In some of these prosthetic socket embodiments, the ischial seat pad is one component of an adjustable-height medial strut cap assembly of the socket, wherein the strut cap assembly further includes a strut cap base attached to and supporting the ischial seat pad for adjustably coupling the ischial seat pad with the medial strut. In particular examples of these embodiments, in a modular manner, the strut cap base is drawn from a collection of strut cap bases that includes multiple different sizes and shapes of strut cap bases configured to fit over multiple sizes and shapes of medial struts. And in other particular examples, in a modular manner, the ischial seat pad is drawn from a collection of ischial seat pads that includes multiple different sizes and shapes of ischial seat pads.

Some of these prosthetic socket embodiments further include a strut cap base for coupling the ischial seat pad with the medial strut, the strut cap base including an adjustable mechanism that allows a vertical position of the strut cap base relative to the medial strut to be adjusted and locked. In particular examples of these embodiments, the ischial seat pad is horizontally slidable with respect to the strut cap base.

In some of these prosthetic socket embodiments, the strut cap base fits over the proximal end of the medial strut; the strut cap base including an internal face, an external face, a proximal end, and a distal end having an opening to accommodate the proximal end of the medial strut. In these embodiments, the ischial seat pad fits over the proximal end of the strut cap base and includes a contoured proximal ischium-contacting face, a distally extending internal portion, and a distal surface configured to align against the proximal end of the strut cap base.

Some of these prosthetic socket embodiments further include a sensor in the ischial seat pad for sensing an amount of force exerted on the ischial seat pad by the patient during use of the prosthetic socket.

In another aspect, embodiments of the invention are directed to a height adjustable strut cap assembly for a prosthetic socket for a residual limb of a lower extremity of a patient. Such a height adjustable strut cap assembly includes a strut cap base including an adjustable, lockable attachment mechanism for attaching to a medial strut of the prosthetic socket such that the strut cap base is vertically adjustable relative to the medial strut and is configured to be locked in position at a desired position relative to the medial strut. Such a height adjustable strut cap assembly further includes an ischial seat pad including a distal surface configured to be securable to a proximal aspect of the strut cap base and a proximal surface configured to engage an ischium of a patient wearing the prosthetic socket.

Some embodiments of the height adjustable strut cap assembly include an open distal end sized and configured to fit over a proximal end of the medial strut. And in some embodiments, the adjustment mechanism of the height adjustable assembly includes a friction-based arrangement such that the strut cap base can be releasably pressed against the medial strut, wherein in the absence of friction the strut cap base can move vertically with respect the medial strut, and wherein when friction is applied, the strut cap base is locked into a fixed vertical position on the medial strut.

Particular embodiments of the height adjustable strut cap assembly further include a sensor in or proximal the ischial seat pad for sensing an amount of force exerted on the ischial seat pad by the patient during use of the prosthetic socket.

Some embodiments of the height adjustable strut cap assembly, the ischial seat pad includes a contoured proximal ischium-contacting face, a distally extending internal portion sized and configured to align against an internal face of a proximal portion of the medial strut, and a distal surface configured to align against the proximal end of the strut cap base.

In another aspect, embodiments of the invention are directed to a method for generating a computer-aided design (CAD) model of an ischial seat pad for a prosthetic socket that is suitable for 3D printing, such CAD model including a constant portion and a variable portion. These method embodiments include generating a constant portion of the CAD model of the ischial seat pad, wherein the constant portion includes a shape complementary to a mounting element configured to allow mounting of the ischial seat pad on the prosthetic socket. Embodiments may further include generating a variable portion of the CAD model of the ischial seat pad, wherein the variable portion is variable according to at least one of quantitative or geometric parameters within each of a series of shaping steps that are applied to a founding central vertical cross sectional profile for an ischial seat pad model, the central vertical cross sectional profile having a proximal surface with an angle of inclination. Such a founding central vertical cross sectional profile may be drawn from a collection of central vertical cross sectional profiles of multiple different sizes and shapes.

In some embodiments of this method of generating a CAD model, generating the variable portion includes applying the series of shaping steps, and wherein the series of shaping steps includes any one or more the following steps: establishing the founding central vertical cross sectional profile for the ischial seat pad model; extruding bilaterally symmetrically from the central cross sectional profile to create an in-progress 3D model of the ischial seat pad; filleting a rectangular external face of the ischial seat pad model being prepared to create a contoured external face on the in-progress 3D model; cutting a radially aligned scallop in the proximal surface of the in-progress 3D model; filleting an internal-facing edge of the contoured scallop of the in-progress 3D model so as to smooth a transition into the radially aligned scallop; filleting a distal internal rectangular aspect of the in-progress 3D model to create a contoured distal edge; contouring right angle corners on a periphery of a proximal surface of the in-progress 3D model; contouring right angle corners on a periphery of a distal surface of the in-progress 3D model; and cutting a distally open laterally aligned pocket on the distal surface of the ischial seat pad to complete the 3D model of the ischial seat pad.

Some embodiment of the method of generating a CAD model further include manufacturing the ischial seat pad by printing, using 3D printing technology, the ischial seat pad from the 3D model. Particular embodiments may further include repeating the series of shaping steps while varying at least one of a dimensionality or an angulation of at least one of the series of shaping steps to provide a different 3D model for a different ischial seat pad. Such embodiments may further include manufacturing the ischial seat pad and the different ischial seat pad by printing, using 3D printing technology, the ischial seat pad and the different ischial seat pad from the 3D model and the different 3D model, respectively.

In yet another aspect, embodiments of the invention are directed to a method for manufacturing an ischial seat pad by way of an ischial seat pad model for a prosthetic socket that is suitable for 3D printing, the method. Embodiments of the method include generating a constant portion of the CAD model of the ischial seat pad, wherein the constant portion comprises a shape complementary to a mounting element configured to allow mounting of the ischial seat pad on the prosthetic socket. Embodiments of the method further include generating a variable portion of the CAD model of the ischial seat pad, wherein the variable portion is variable according to at least one of quantitative or geometric parameters within each of a series of shaping steps that are applied to a founding central vertical cross sectional profile for an ischial seat pad model, the central vertical cross sectional profile having a proximal surface with an angle of inclination. Embodiments of the method conclude by using the CAD model to manufacture the ischial seat pad.

In particular embodiments of the method, using the CAD model includes printing the ischial seat pad from the CAD model using a 3D printing technology. Embodiments of the method, before generating the variable portion of the CAD model, may include acquiring a 3D digital profile of a region of a patient's pelvis surrounding an ischial tuberosity in the form of an STL file; and importing the STL file into a CAD application for further processing. By this approach, a CAD model for an ischial seat pad may be custom made to complement the ischial tuberosity and surrounding region of the patient's pelvis in order to optimize the fit of the ischial seat pad to the patient.

These and other aspects and embodiments are described more fully below, in reference to the attached drawing figures.

### BRIEF DESCRIPTION OF DRAWINGS

FIGS. 1A-1D are top perspective, side, bottom and bottom perspective views, respectively, of a distal base of a modular prosthetic socket, with a default offset distal connection site, according to one embodiment;

FIG. 1E is a bottom perspective view of the distal base of FIGS. 1A-1D with a strut connector attached to the top (proximal) surface of the distal base, according to one embodiment;

FIG. 1F is a cross-sectional side view of the distal base of FIGS. 1A-1E, showing the longitudinal axis of the distal element hosting receptacle to be flexed at an angle of 7° with respect to the longitudinal axis of the base as a whole;

FIGS. 2A-2C are top perspective, bottom perspective and bottom views, respectively, of a distal base embodiment with a zero offset distal connection site, according to one embodiment;

FIG. 3A is a top perspective view of a strut connector of a modular prosthetic socket, according to one embodiment;

FIGS. 3B and 3C are top perspective views of the strut connector of FIG. 3A, with a pivot tab in place within a wedge-shaped recess of the strut connector, the pivot tab positioned to one side and centered, respectively, in FIGS. 3B and 3C, according to one embodiment;

FIGS. 3D-3H are front, side, rear, bottom and top views, respectively, of the strut connector of FIG. 3A;

FIGS. 3I-3J are top views of the strut connector and pivot tab of FIGS. 3B and 3C;

FIGS. 3K-3N are top perspective, front, side and back views, respectively, of the strut connector of FIGS. 3A-3J, with the pivot tab in place and centered within a wedge-shaped recess of the base portion of the strut connector and a strut disposed within the strut connector and secured by three bolts, according to one embodiment;

FIG. 4A is a perspective, exploded view of a modular prosthetic socket, with four longitudinal struts differentiated by their circumferential positions—a medial or ischial strut, an anterior strut, a lateral strut, and a posterior strut, according to one embodiment;

FIG. 4B is a perspective, assembled view of the modular prosthetic socket of FIG. 4A;

FIGS. 5A and 5B are perspective and top views of a distal end of a thermoplastic fiber composite strut of a modular prosthetic socket, which includes a longitudinally aligned concavity, according to one embodiment;

FIGS. 6A-6C are top perspective, bottom perspective and bottom views of a distal end of a strut of a modular prosthetic socket, with metal cladding pieces attached to internal and external surfaces of the distal end, according to one embodiment;

FIGS. 7A-7M are rear (7A), top (7B), rear perspective (7C), front perspective (7D), rear perspective (7E), side (7F), cross-sectional side (7G), cross-sectional top (7H), rear exploded (7I), side exploded (7J), cross-sectional side exploded (7K), front perspective exploded (7L), and rear perspective exploded (7M), views, respectively, of an adjustable ischial strut cap assembly, according to one embodiment;

FIGS. 7N and 7O are both rear perspective views of a modular prosthetic socket, including the adjustable ischial strut cap assembly of FIGS. 7A-7M, showing a user loosening the adjustable height locking mechanism, which allows the strut cap assembly and seat pad to telescopically adjust up or down, according to one embodiment;

FIGS. 7P-7Q show an adjustable height ischial strut cap assembly in an elevated height position (FIG. 7P) and in a low-height position (FIG. 7Q).

FIGS. 7R-7S show an embodiment of an ischial strut cap assembly that is equipped with a force sensor and microprocessor and transmitter unit;

FIGS. 7T-7U show an embodiment of an ischial strut cap assembly in which the ischial seat is horizontally slidable with respect to the ischial strut cap base supporting it;

FIG. 7V shows a face view of an ischial seat pad that includes non-Newtonian foam disposed proximally, along the ischium-contacting surface.

FIG. 7W shows a vertical cross sectional view of an ischial seat pad that includes a temperature controlled material.

FIG. 7X shows a transparent face view of an ischial seat pad that includes an inflatable air bladder.

FIG. 8A is a side view of a seat pad for an ischial strut cap assembly for a modular prosthetic socket, according to one embodiment;

FIGS. 8B-8E are internal perspective views of various strut cap embodiments: in columns from left to right, strut cap embodiments having (1) a simple surface (FIG. 8B), (2) a concave surface (FIG. 8C), (3) a high medial wall, the left-side version (FIG. 8D), and (4) a high medial wall, the right side version (FIG. 8E). Rows 1 and 2 of columns 1 and 2 show, from top to bottom, a base-to-ischium contacting surface tangent angle of 5°, 15°, 25°, and 35°. Rows 3 and 4 show, from top to bottom, strut cap widths of 60 mm, 70 mm, and 80 mm, according to various embodiments;

FIGS. 8F-8I are external perspective views of the same embodiments, shown in the same order, of FIGS. 8B-8E;

FIGS. 8J-8M are side views of the same embodiments, shown in the same order, of FIGS. 8B-8I;

FIGS. 8N-8Q are internal face views of the same embodiments, shown in the same order, of FIGS. 8B-8M;

FIGS. 8R-8Z are face perspective, internal perspective, face perspective, external perspective, top, cross-sectional side, bottom, top and side views, respectively, of a seat pad that is formed from two materials, one with a durometer of about 95D (hard) and one with a durometer of about 50D-60D, according to one embodiment;

FIG. 9A is a perspective view of a modular prosthetic socket being worn by a patient, according to one embodiment;

FIGS. 9B-9E are anterior/lateral, lateral, posterior and medial views, respectively, of the modular prosthetic socket of FIG. 9A;

FIG. 9F shows the modular prosthetic socket of FIGS. 9A-9E, with the components disassembled, the components including a brim (butterfly wrapping segment, trochanteric pad segment, adjustable ischial or medial strut cap assembly with seat pad, tensioning belt with ratchet buckle), four struts, four strut sleeves, a distal base, and a flexible distal cup with an anchoring base, according to one embodiment;

FIGS. 10A-10J are anterior, medial, posterior, lateral, top anterior perspective (left), top posterior perspective (right), bottom anterior perspective (left), bottom posterior perspective (right), top face, and bottom face views, respectively, of a proximal brim member for a modular prosthetic socket, according to one embodiment;

FIGS. 11A and 11B are front and side views, respectively, of a butterfly wrapping component of a prosthetic socket brim member in a laid out flat position, according to one embodiment;

FIGS. 11C and 11D are front and cross-sectional side views, respectively, of a trochanteric pad component of a prosthetic socket brim member, according to one embodiment;

FIG. 12A is a perspective, partially cutaway view of flexible distal cup with an anchoring plate bonded to distal end of the flexible distal cup, according to one embodiment. A 2-way valve is shown on the left (medial side of liner); a 1-way expulsion valve is shown on the right (lateral side of liner);

FIG. 12B is a top view, looking down into flexible distal cup of FIG. 12A, rendered transparently, and an anchoring plate, partially exposed, at the distal end of the flexible inner liner. A 2-way valve is shown on the left (medial side of liner); a 1-way expulsion valve on the right (lateral side of liner);

FIG. 12C is a bottom view of the flexible distal cup anchoring plate at distal end of a flexible distal cup of FIG. 12A;

FIG. 12D is a bottom perspective view of the anchoring plate of FIG. 12A;

FIG. 12E is a close-up bottom perspective view of the anchoring plate at the distal end of a flexible distal cup of FIG. 12A;

FIG. 12F is a close-up bottom perspective view of the anchoring plate at distal end of a flexible distal cup of FIG. 12A;

FIG. 12G is a longitudinal cross sectional view of the flexible distal cup with the anchoring plate bonded at the distal end of FIG. 12A. Three connecting pedestals are shown, medial, lateral, and central, according to one embodiment;

FIG. 13A is a medial side view of a flexible distal cup situated within a modular prosthetic socket, according to one embodiment;

FIG. 13B is a top view of the flexible distal cup and modular prosthetic socket of FIG. 13A, with the anterior aspect of the flexible distal cup at the 6 o'clock position;

FIG. 13C is a posterior side perspective view of the flexible distal cup and modular prosthetic socket of FIG. 13A;

FIG. 13D is a bottom view of the flexible distal cup and modular prosthetic socket of FIG. 13A, with the anterior aspect of the flexible distal cup at the 6 o'clock position;

FIG. 13E is a top perspective view of the flexible distal cup and modular prosthetic socket of FIG. 13A;

FIG. 13F is a lateral side view of the flexible distal cup and modular prosthetic socket of FIG. 13A, with the posterior projecting portion of the base plate on the right;

FIG. 13G is a medial side face view of the flexible distal cup and modular prosthetic socket of FIG. 13A, with the posterior projecting portion of the base plate on the left;

FIG. 13H is a longitudinal cross sectional view of a distal portion of the flexible distal cup and modular prosthetic socket of FIG. 13A, the flexible distal cup being supported on a distal base and within a set of struts;

FIG. 14 shows a central cross sectional of a CAD model of an ischial seat model that has a constant portion and a variable portion;

FIGS. 15A-15I show ischial seat pad models as they progress through a series of shaping steps: FIG. 15A shows an initial step of establishing a founding central vertical cross sectional profile for an ischial seat pad model;

FIG. 15B shows a step of extruding bilaterally symmetrically from the central cross sectional profile of the ischial seat pad model being prepared;

FIG. 15C shows a step of filleting the rectangular external face of the ischial seat pad model being prepared to create a contoured external face;

FIG. 15D shows a step of cutting a radially aligned scallop in the proximal surface of the ischial seat pad model being prepared;

FIG. 15E shows a step of filleting the internal-facing edge of the contoured scallop so as to smooth transition into the radially aligned scallop;

FIG. 15F shows a step of filleting a distal internal rectangular aspect of the ischial seat pad to create a contoured distal edge;

FIG. 15G shows a step of contouring the right angle corners on the periphery of the proximal surface of the ischial seat pad being prepared;

FIG. 15H shows a step of contouring the right angle corners on the periphery of the distal surface of the ischial seat pad being prepared;

FIG. 15I shows a step of cutting a distally open laterally aligned pocket on the distal surface of the ischial seat pad being prepared;

FIGS. 15J-15L show various approaches to creating regions of variable fill in a 3D printed ischial seat, and accordingly creating regions of varied durometer: FIG. 15J shows an ischial seat pad wherein variable fill density is created by gradually varying the distribution of sized void volumes within printed thermoplastic media;

FIG. 15K shows an ischial seat pad with a proximal region of low thermoplastic media fill density and a distal region of high fill density;

FIG. 15L shows an ischial seat pad with an internal region of homogenous fill density and a proximal skin surface of high fill density;

FIG. 16 is a flow diagram of an embodiment of a method of creating a CAD model of an ischial seat pad suitable for driving a 3D printing process to print an embodiment of an ischial seat pad.

FIG. 17 is a flow diagram of an embodiment of a method of creating an inventory of CAD models of an ischial seat pad that vary in form that are suitable for 3D printing of an inventory of ischial seat pads.

FIG. 18 is a flow diagram of an embodiment of a method of preparing an integral model of an ischial seat pad suitable for driving a 3D printing process

FIG. 19A shows a top perspective view of a circumferentially configured laminated prosthetic socket with a height adjustable ischial seat disposed on its medial aspect.

FIG. 19B shows a side view of a circumferentially configured laminated prosthetic socket with a height adjustable ischial seat disposed on its medial aspect.

### DETAILED DESCRIPTION

The present disclosure relates to modular prosthetic sockets and components thereof. Sections that follow below include: (A) embodiments of prosthetic socket hardware, (B) embodiments of soft goods that serve as rigging for hardware components, (C) a composite polymer material and a flexible distal cup fabricated therefrom, (D) improvements in technology related to translating digital residual limb data (size, shape, and tissue density) into custom, patient-specific shapes for assembly into a modular prosthetic socket, and (E) custom fitting of an ischial seat pad and methods of fabricating via 3D printing technology.

### A. Hardware Components

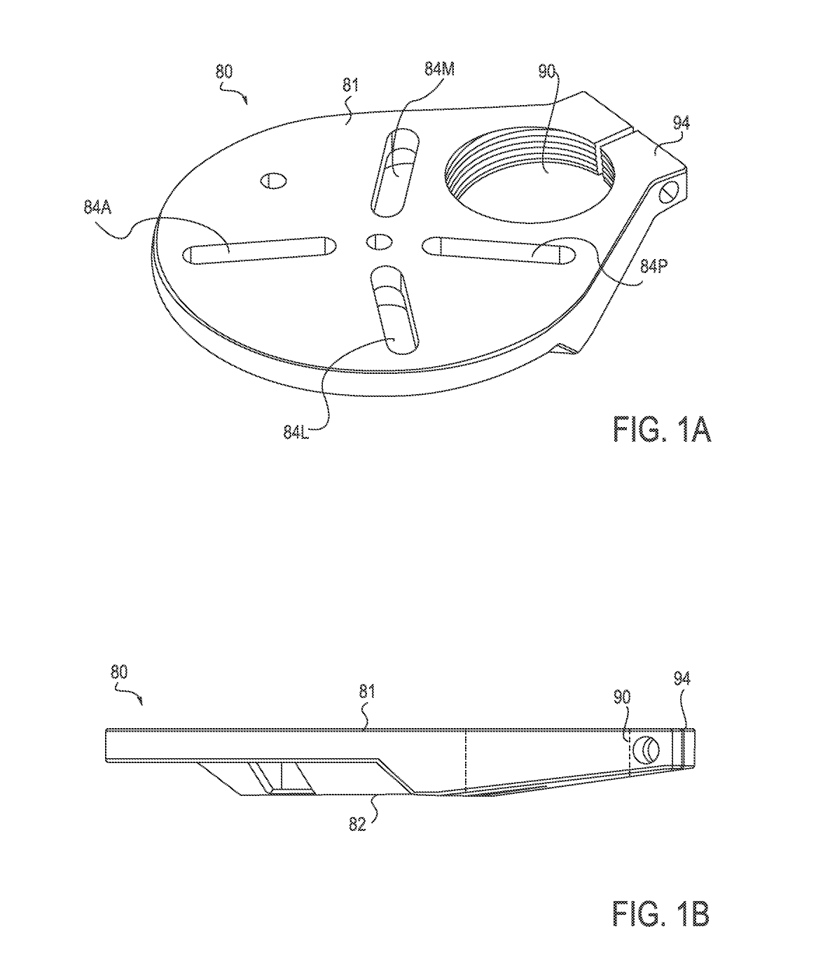

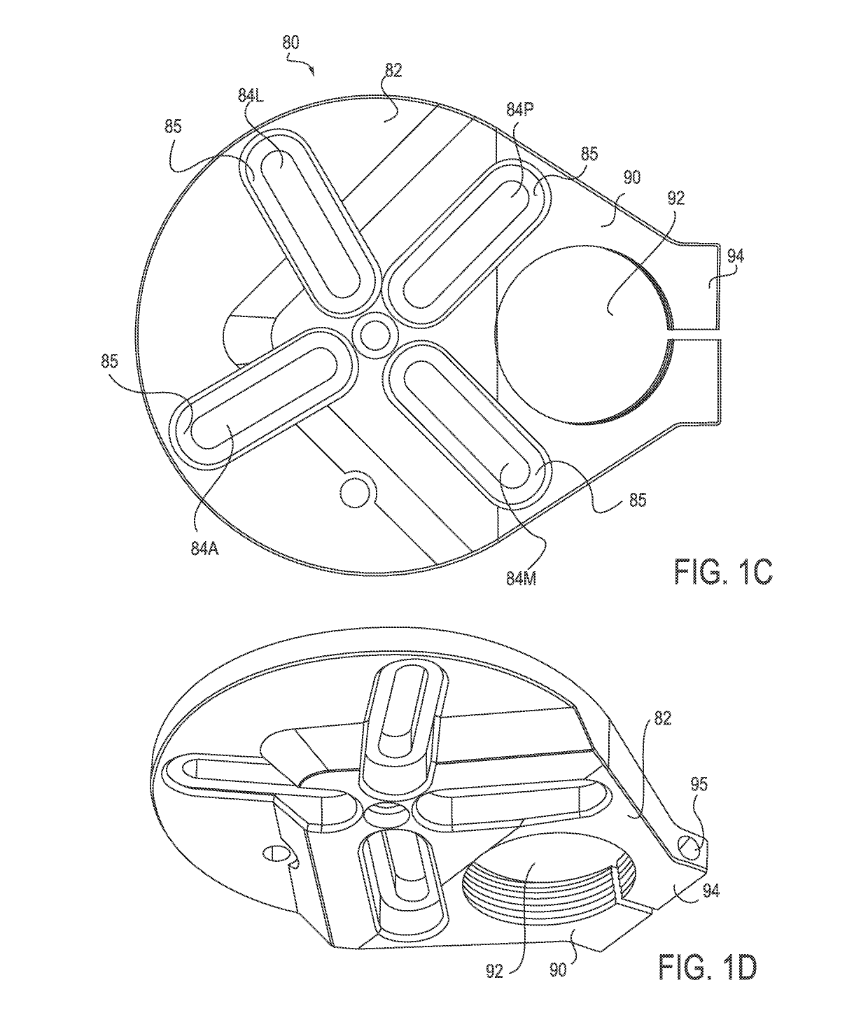

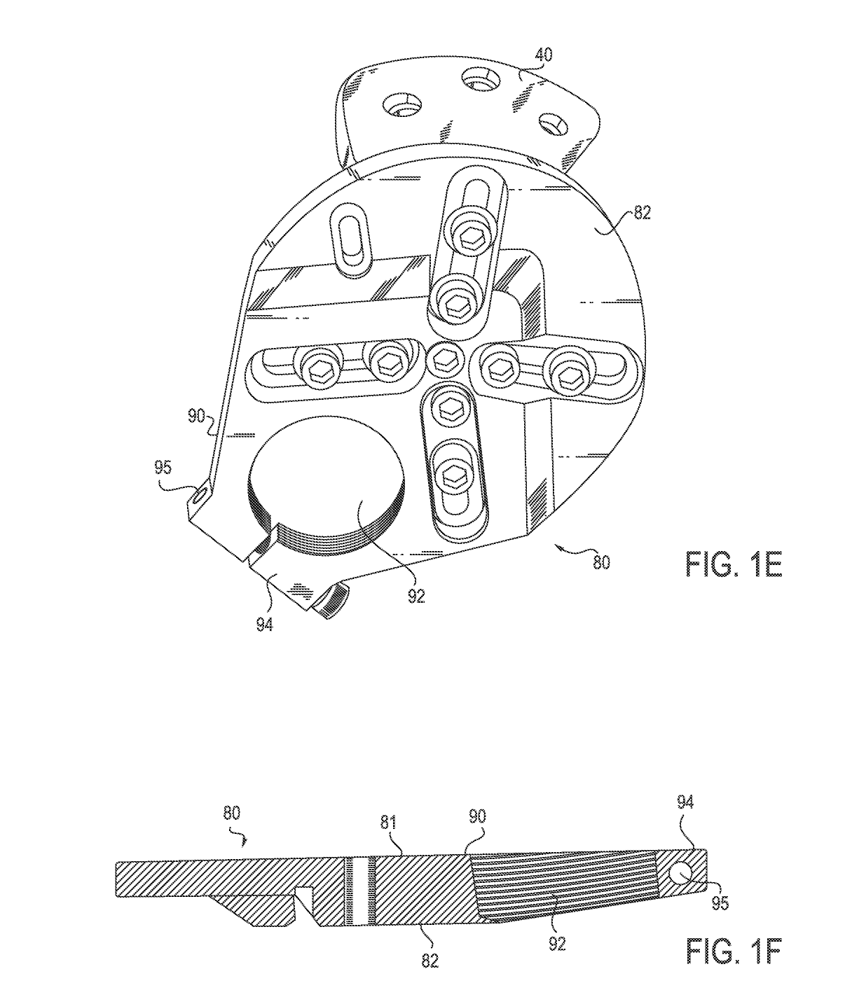

The technology provided herein relates to new components, features, and improvements for a modular prosthetic socket device and system, as described in U.S. patent application Ser. Nos. 13/675,761 and 14/213,788, which were previously incorporated by reference. New features and improvements may be divided into two categories—hardware and soft goods—which together form a modular prosthetic socket system. “Hardware” (or “hard goods”) refers to modular components that form the prosthetic socket structural frame, such as distal base plate embodiments, strut connector embodiments, longitudinal strut embodiments, and telescoping adjustable ischial strut cap embodiments. Some embodiments relate to previously described components provided in the referenced patent applications, and other embodiments are described in the present patent application for the first time. Aspects and embodiments of presently described components are depicted in FIGS. 1A-13H.

Modularity or modular assembly refers to the use of components that are provided as groups, or inventories of like components, broadly similar in structure. These structurally similar components vary in some aspect of form or size, but retain common aspects that allow them to be assembled together, regardless of such variation in form or size. The overall goal of such an approach to assembling a modular prosthetic socket is to be able to create a highly customized product, specific for each individual patient, from a manageable group of components. As their category designation indicates, hardware components are typically hard materials such as metal, plastics, or thermoplastic-fiber composite materials. A distal cup is also described in this present application, which is formed from a composite thermoplastic material. Although a distal cup may be considered to be a soft good type of component, not as “structural” in the sense that struts are, it nevertheless can be modular, varying in shape and/or size, and provided in an inventory.

Some modular components of a prosthetic socket 10, such as a distal base 80 or a strut connector 40 are fabricated from metal, and are thus substantially fixed in form, once fabricated. Other modular components, such as thermoplastic fiber composite struts 20 or 24 and a flexible distal cup 200, described herein, are thermally reformable after their initial fabrication. Thus, a modular assembly may be a hybrid of two types of components, some of which are prefabricated in a fixed (albeit variable) form, and some of which are heat reformable, and thus customizable into individually bespoke forms (i.e., forms that are not included in an inventory).

Customization of the fit of a modular prosthetic socket 10 to an individual patient, accordingly, occurs through one or more approaches: (1) selection of appropriately sized and shaped fixed form components; and (2) selection of appropriately sized and shaped thermally reformable components. Fixed form components may include, by way of example, a distal base or a strut connector. A thermally reformable component is selected from an inventory to provide at least a first approximation of fit, and then may be reformed to optimize fit. Such components may include a strut or a flexible distal cup. Some embodiments of a brim 100, being formed from low density polyethylene (LDPE) may also be thermally reformable.

Soft goods, in contrast, are made of fabrics, soft plastic components, and associated connecting and tensioning mechanisms. Soft goods generally serve as elements that connect hardware components together, acting as pressure-distributing elements, in conjunction with hardware, distributing pressure away from focal sites of contact between the hardware and the residual limb. Soft goods are described in detail below.

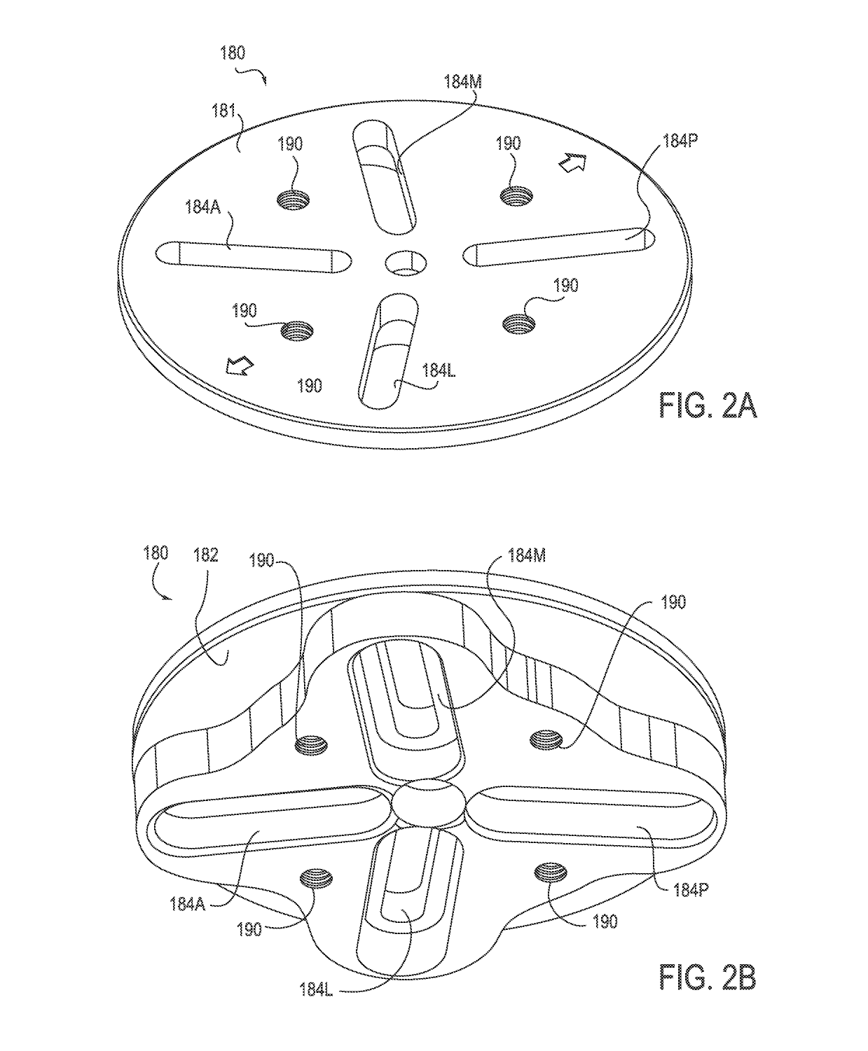

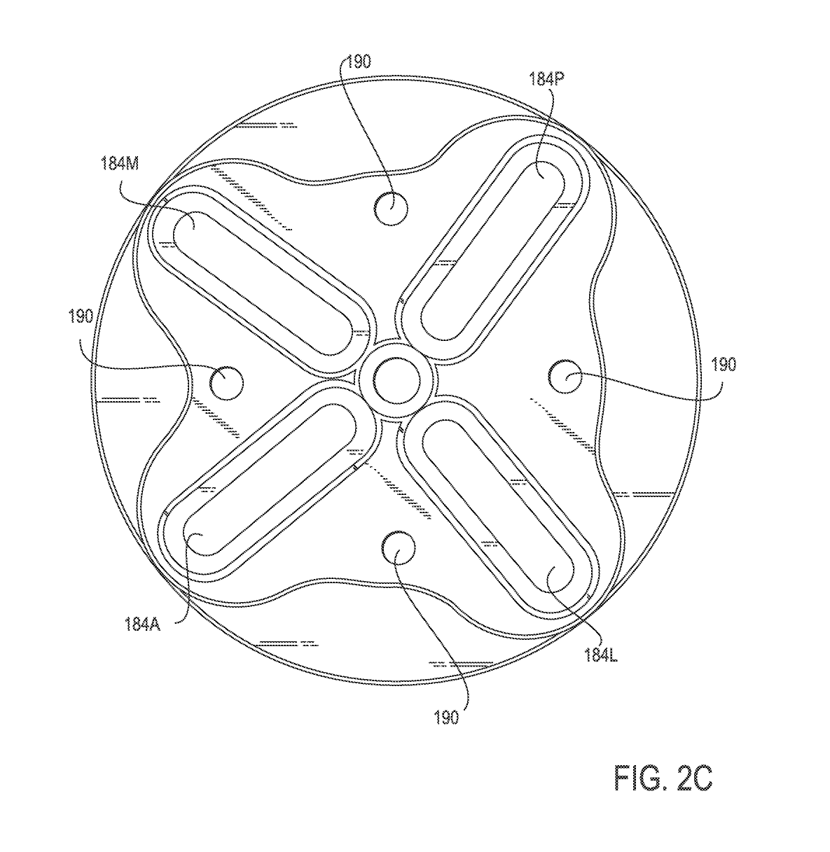

U.S. patent application Ser. Nos. 13/675,761 and 14/213,788 describe various aspects and embodiments of a distal base for a modular prosthetic socket. For example, various distal base embodiments are depicted in FIGS. 2A, 15A-15G, and 17A-19B of U.S. patent application Ser. No. 14/213,788. The present application describes one embodiment of a distal base 80 in relation to FIGS. 1A-1F and an alternative distal base embodiment 180 in relation to FIGS. 2A-2C. The distal bases 80, 180 are compatible with modular prosthetic socket struts 20 coupled with strut connectors 40. Distal bases 80, 180 are also compatible with modular prosthetic socket strut 24 that include metal cladded elements 26-I and 26-E. Both types of struts are described in detail below.

### Distal Base

Referring now to FIGS. 1A-1F, distal base 80 for a modular prosthetic socket is a single (or “unitary”) base plate, with a proximal surface 81, a distal surface 82, and a distal prosthetic component mounting portion 90. In some embodiments, the distal prosthetic component mounting portion 90 includes a circular through hole or receptacle 92 that is configured to host a distally projecting distal prosthetic element (not shown). Portion 90 further includes a compressible split annular feature 94 (and a bolt hole 95 therethrough) within the circular profile, which is compressible to secure the distal prosthetic element. This split annular feature 94 of receptacle 92 may also be understood as a rotational lock, in that it is able, by its closure via bolt 96, to lock a male threaded portion of a distal prosthetic element within the receptacle 92. The distal prosthetic component mounting portion 90 of distal base 80 has a default off-center position.

Distal base 80 occupies a central structural role in a prosthesis, serving both as a distal base for a proximal structure (modular prosthetic socket 10), as well as a proximal base for distal portions of a complete prosthesis (not shown). Further, distal base 80 serves an aligning function, as proximal and distal prosthetic structures are not collinear. Thus, distal base 80 is subject to considerable stress, and a material with a high strength/density ratio is desirable. Accordingly, in some particular embodiments, distal base 80 is fabricated from 7075 t6 aluminum.

It is helpful in understanding the spatial orientation of distal base 80 to refer to the major planes of the residual limb and a modular prosthetic socket 10 disposed on the limb, and to relate those planes to aspects of distal base 80. Accordingly, distal base 80, as a substantially flat plate, generally occupies a transverse plane. The coronal plane provides lateral and medial reference directions applicable to edges, or general aspects of the distal base 80. The sagittal plane similarly provides anterior and posterior reference directions to applicable to distal base 80 edges or regions. By this approach, an anterior-posterior/medial-lateral (AP/ML) map can be applied to distal base 80.

With these general orienting references, in some distal base embodiments 80, the distal element prosthetic component mounting portion 90 occupies a generally posterior position on distal base 80, and generally provides it a non-circular asymmetric face profile. “Distal prosthetic element”, in this context, refers to any prosthetic element beyond or below the prosthetic socket, such as a pylon or a knee. Further details of distal prosthetic component mounting portion 90 and circular receptacle 92 are provided below.

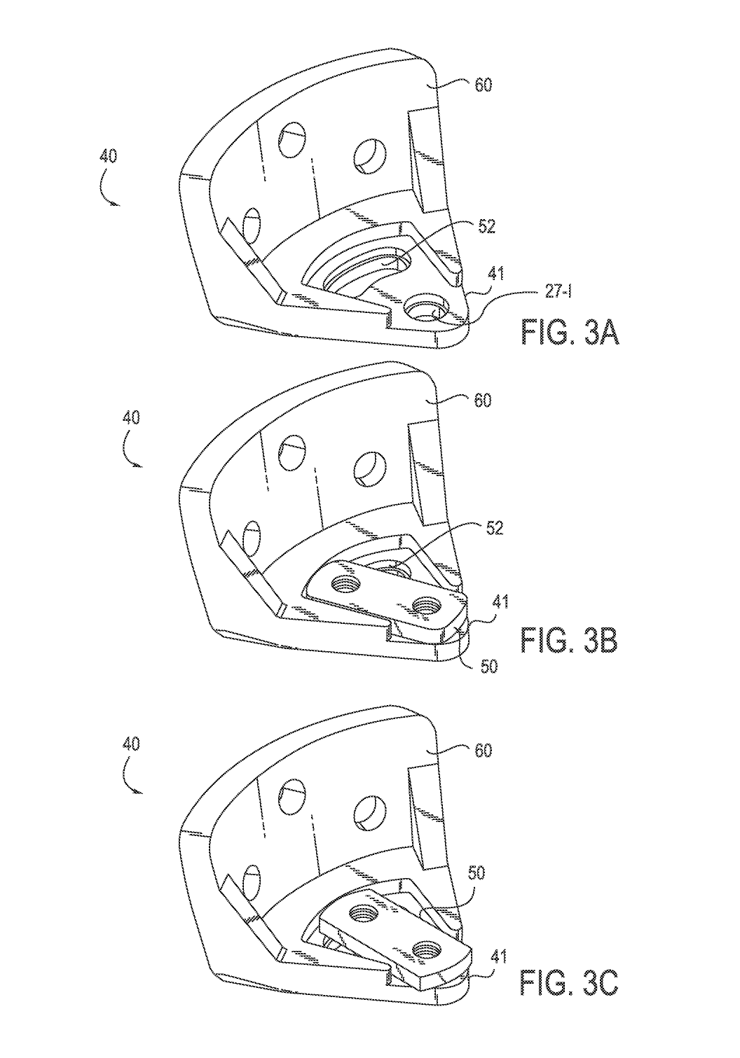

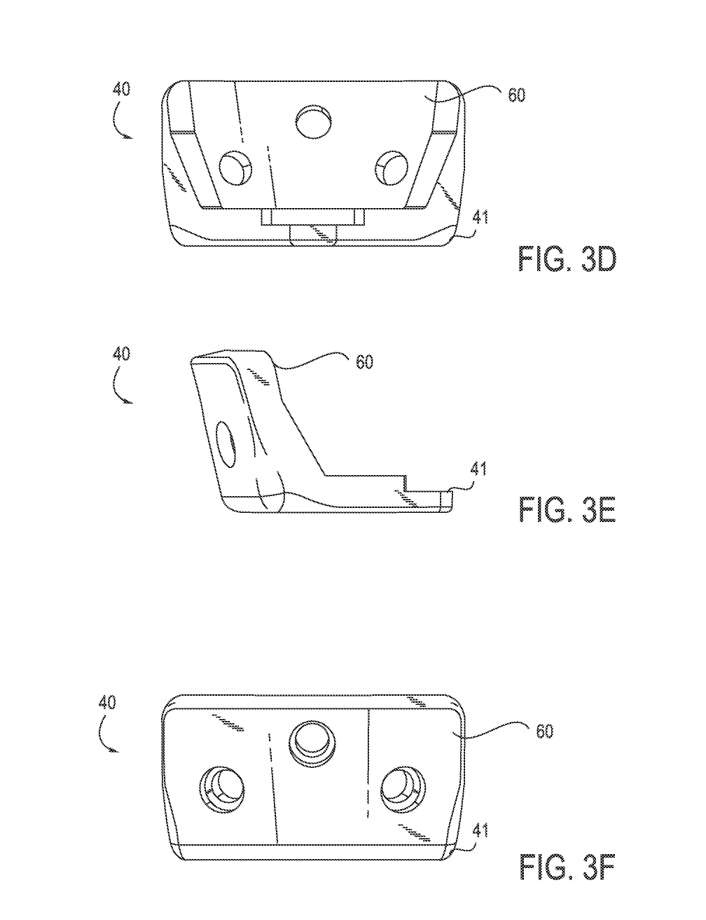

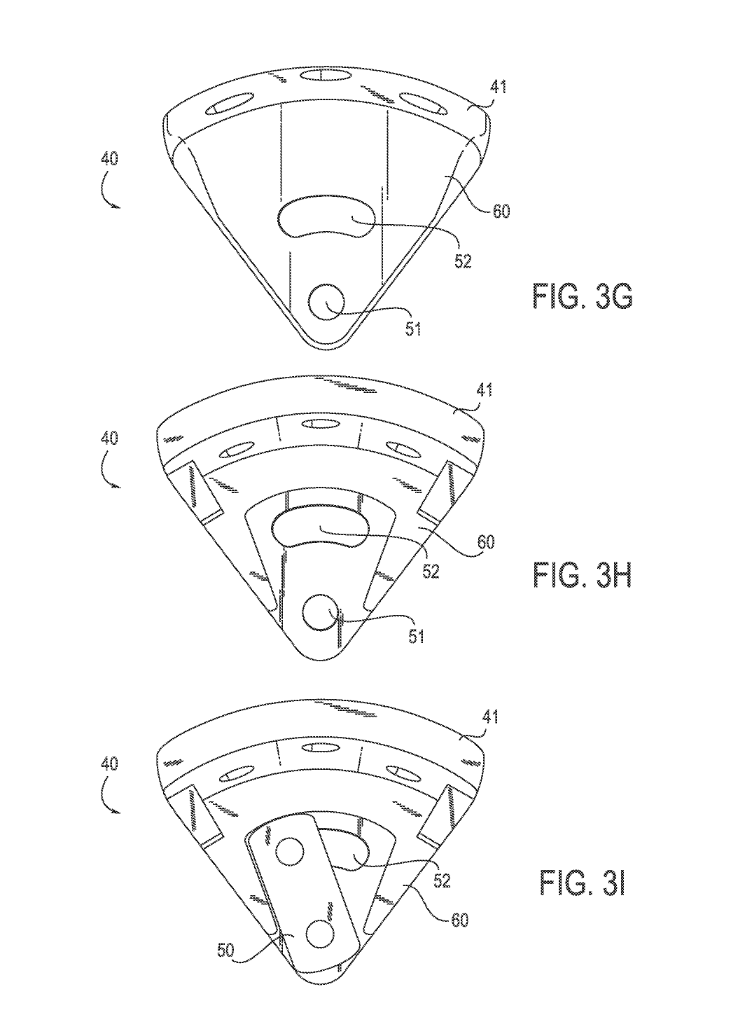

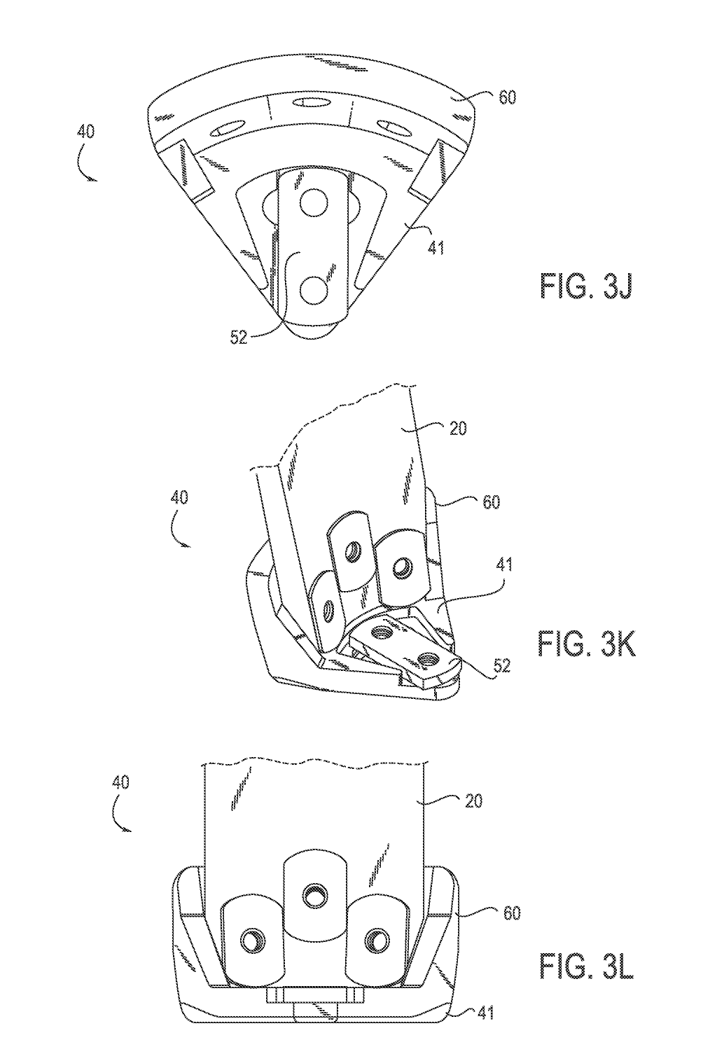

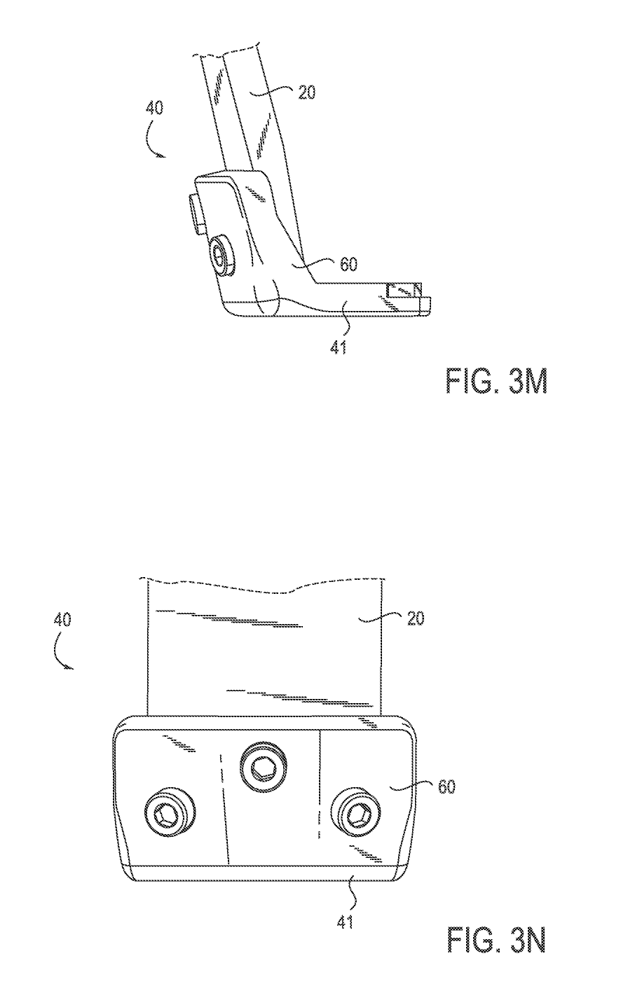

Turning briefly to multiple longitudinal struts 20 (e.g., FIGS. 3K-3N), to provide context for peripherally-disposed strut connecting sites, such as connector slots 84, within distal base 80: Embodiments of a modular prosthetic socket 10, as provided herein, include multiple longitudinal struts 20, typically three struts or four struts, disposed proximal to distal base 80. Accordingly, some embodiments of distal base 80 include three or four radially oriented through-slots 84 that are configured to slidably host an equivalent number of strut connectors 40. Exemplary prosthetic socket 10 embodiments, as provided herein, have four struts 20 and four hosting through-slots 84 within distal base 80. Radially oriented slots 84 are peripherally disposed around distal base 80 and, accordingly, may be more generally referred to as peripherally disposed strut connecting sites.

The angular distribution of through-slots 84 may be arranged in any suitable manner to accommodate a desired circumferential distribution of struts 20. Label identifier 20 refers to any strut generally, without reference to its location when assembled into a modular prosthetic socket 10. However, when assembled, struts 20 may be identified by their circumferential position, according to the (AP/ML) map referenced above. For example, struts 20 may be identified as a medial strut 20M (or “ischial strut”), anterior lateral strut 20AL, anterior medial strut 20AM, and posterior strut 20P. Similarly, label identifier 84 refers to any strut connector slot within distal base 80, but strut connector slots may also be identified according to their relative position according to the AP/ML mapping reference as follows: medial (ischial) strut connector slot 84M, anterior lateral strut connector slot 84AL, anterior medial strut connector slot 84AM, and posterior strut connector slot 84P.

As noted with regard to the angular distribution of struts, strut connector slots 84 in distal base 80 may be configured in any suitable manner. It is common, however, for the angular gap between struts on the medial aspect of a residual limb to be larger than other gaps. Accordingly, in one particular embodiment of distal base 80, the angular gaps between radially oriented through strut connector slots 84 are as follows:

| inter-slot space | region of the base | angle |
|---|---|---|
| Slot 84M - Slot 84P | Medial-posterior | 90° |
| Slot 84P - Slot 84AL | Posterior-lateral | 75° |
| Slot 84AL - Slot 84AM | Anterior-lateral | 90° |
| Slot 84AM - Slot 84M | Anterior-medial | 105° |

When strut connectors 40 are assembled onto distal base 80, bolts are inserted into strut connector slots 84 from the distal aspect 82 of base 80, pulling the strut connector down directly onto the proximal surface 81 of base 80. In some embodiments, the internal aspect of strut connector slots 84 includes a shelf 85 that is configured such that the head of the bolt, when tightened against the shelf, does not project beyond the distal surface 82 of distal base 80.

Returning now to distal prosthetic element mounting portion 90 and circular receptacle 92 that is configured to host a distally projecting prosthetic element and its position on distal base 80 with reference to an AP/ML map: Whereas the central aspect of distal base 80 is functionally directed proximally, anchoring structural elements of a modular prosthetic socket 10, this distal prosthetic element mounting portion 90 is functionally directed to anchoring the distal portion of the prosthesis and appropriately aligning the distal prosthesis with respect to the socket. Circular receptacle 92, in some embodiments, is 36 mm in diameter, with a thread of 1.5 mm pitch. This configuration is a standard arrangement for distal prosthetic component connections.

As noted earlier, receptacle 92 is generally disposed within the posterior aspect of distal base 80. More particularly, a center of receptacle 92 may be located between about 2.5° and about 12.5° lateral to a 0° (posterior) reference line. In one particular embodiment, the center of receptacle 92 is located at about 7.5° lateral from a 0° (posterior) reference line. In terms of center-center distance between the center of distal base 80 and center of receptacle 92, such offset distance in various embodiments can vary between about 30 mm and about 40 mm. In particular embodiments, the offset distance is about 35 mm. The functional reason for this position relates to the relative position of a distal prosthetic element (e.g., a knee) with respect to the proximally disposed modular prosthetic socket. Typically, the biomechanically appropriate alignment is one in which the distal element is posterior and lateral with respect to the prosthetic socket.

Another aspect of receptacle 92 relates to its longitudinal orientation. Longitudinal, in this context, refers to the longitudinal axis through the center of the receptacle, with respect to the longitudinal axis of modular prosthetic socket 10 as a whole. If the longitudinal axis of the prosthetic socket is used as a 0° reference, the longitudinal orientation of an axis through the center of receptacle 92 may be oriented at a flexion angle of between about 2.5° and about 12.5°. In some embodiments, the flexion angle of the longitudinal axis is about 7.5° with respect to the longitudinal axis of the prosthetic socket (or about 82.5° with respect to the angle of the plane of distal base 80). Further, in some embodiments, the longitudinal axis of receptacle 92 is oriented with about 7° of adduction (i.e., the distal end of the axis pointing toward the center of the body) with respect to the longitudinal axis of the prosthetic socket.

Another adaptive aspect of distal base 80 relates to regions that vary in thickness, having relatively thick regions and relatively thin regions, as may be seen in various bottom and side views. These variations arise as a balance between the desirability for strength within the base structure (calling for thick regions) and the desirability for minimizing weight (calling for thin regions). The distribution of thick regions is arranged to strengthen areas that are subject to stress when the distal base is loaded with force.

Referring now to FIGS. 2A-2C, distal base 180 is a variation of distal base 80 that is similar in many aspects, but differing with regard to the position of the mounting site that allows connection to a distal prosthetic element such as a pylon or a knee. Distal base 80 is configured with a default offset for the mounting site 92 to a distal prosthetic element, while distal base 180 has a centered distal prosthetic connection arrangement. In some embodiments, this connection arrangement is in the form of a standard 4-hole adaptor connection site 190, as seen in FIGS. 2A-2C. A four-hole adapter can connect to a large number of available distal prosthetic elements.

Features of distal base 180 that are analogous or identical to those of distal base 80 include the single or unitary base configuration, and the configuration and angular distribution of the strut slots. The relative appropriateness of distal base 80 or 180 depends on patient-specific alignment factors. Some patients may prefer one distal base alternative over the other, and some patients may be able to use both or either, depending on circumstance.

As described extensively in U.S. patent application Ser. No. 14/213,788, the internal volume and the shape of the space included within a modular prosthetic socket can be adjusted by adjusting the radial position and angle of pivot of strut connectors on a distal base.

As can be seen in FIGS. 1A-1F, bolt 96, which controls the locking of a distal prosthetic element in place within receptacle 92 of distal element mounting portion 90 of distal base 80 is readily accessible when modular prosthetic socket 10 is being worn by a patient. Further, bolts 55, which control the locking of strut connector 40 in strut connector slots 84 or distal base 80 are readily accessible when modular prosthetic socket 10 is being worn by a patient.

Accordingly, one embodiment of a method of adjusting the fit of a modular prosthetic socket 10 to a patient includes adjusting the fit of modular prosthetic socket by loosening any of bolts 55 while the patient is wearing the modular prosthetic socket 10, loosening a bolt, moving a strut connector radially or pivoting a strut connector, and tightening the bolt. In another embodiment of a method directed toward adjusting the alignment of a distal prosthetic element, the method includes loosening bolt 55, adjusting the alignment, and tightening the bolt while the patient is wearing the prosthetic socket.

### Strut Connectors

Referring now to FIGS. 3A-3N, in some embodiments of a modular prosthetic socket, strut connectors 40 connect struts 20 to distal base 80. Strut connectors 40 include a base portion 41 that is connectable to distal base 80 and a back portion 60 to which struts 20 are connected.

The angle between base portion 41 and back portion 60 can vary among embodiments. For example, an inventory of strut connectors could have models with a base-to-back angle of 95°, 110°, 125°, and 135°.

Base portion 41 includes two bolt holes that allow the strut connector to be secured to distal base 70: an inner bolt hole 42, and an outer crescent shaped bolt hole 43. Bolt holes 42 and 43 are both disposed within a wedge shaped recess 45 on the upper aspect of base portion 41. The portion of the upper aspect of base portion 41 situated between crescent shaped hole 43 and back portion 50 includes a strut ledge 48, against which the distal portion of a strut 20 rests when the strut is bolted into the strut connector.

Particular embodiments of strut connectors 20 have a longitudinally aligned inward facing concavity that complements the shape of strut ledge 48 and sits well against the inward facing concave aspect of back portion 60 of strut connector 40.

An insert tab 50 is disposed within wedge shaped recess 47. Bolts 55 connect base portion 41 and insert tab 50 together. These bolts penetrate through inner bolt hole 51 and outer bolt hole 52 within insert tab 50; these bolt holes align with inner bolt hole 42 and outer crescent shaped bolt hole, respectively of base portion 41 of strut connector 40.

Back portion 60 of strut connector 40 includes three bolt holes 62 that align with complementary bolt holes at the distal ends of struts 20. Buttress portions 68 of strut connector 60 are disposed on both sides of strut connector 40, having a triangular shape that joins back portion 60 and base portion 41 together.

Thermoplastic fiber composite struts, useful in the assembly of a modular prosthetic socket, are described and depicted in U.S. patent application Ser. No. 14/213,788, as referenced above. That application has extensive disclosure related to the structure and composition of thermoplastic fiber composite struts and their connections to both proximal and distal components (see paragraphs 18-34, 53-59, 102-123, in particular). That application further has extensive disclosure related to methods of forming and reforming struts (paragraphs 124-144). The present disclosure includes description of new features and embodiments of struts.

### Struts

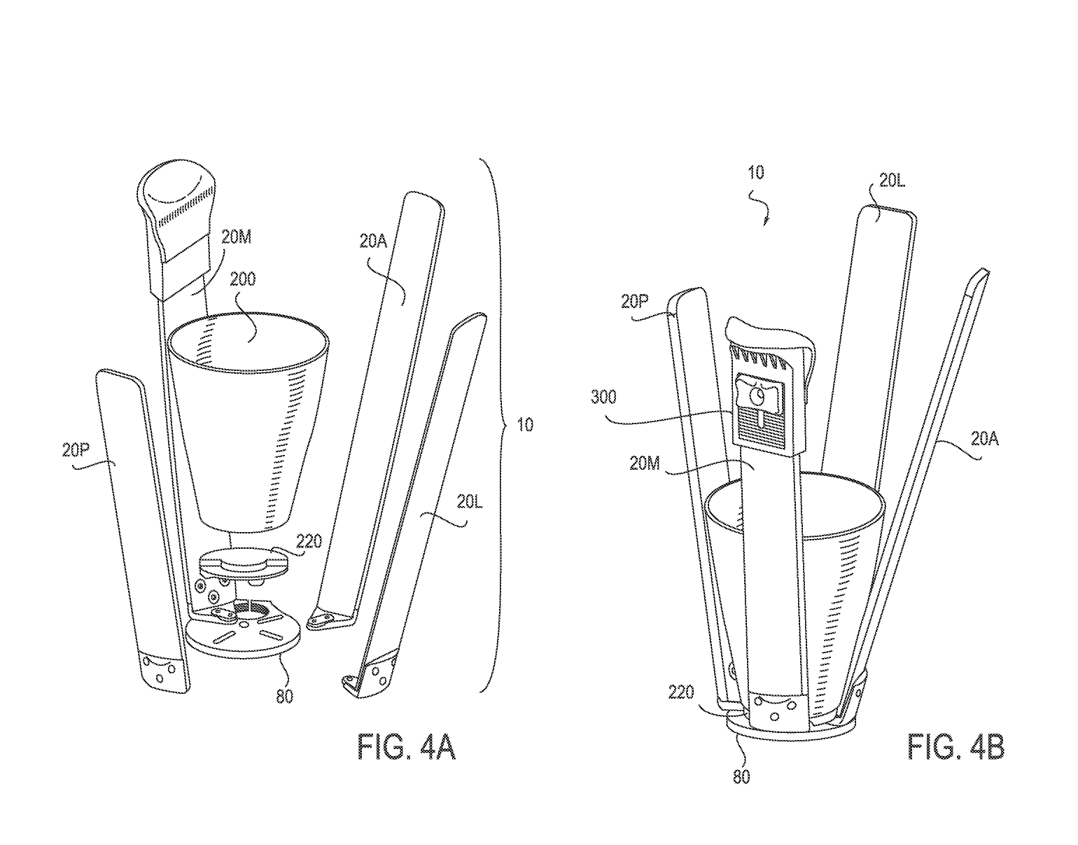

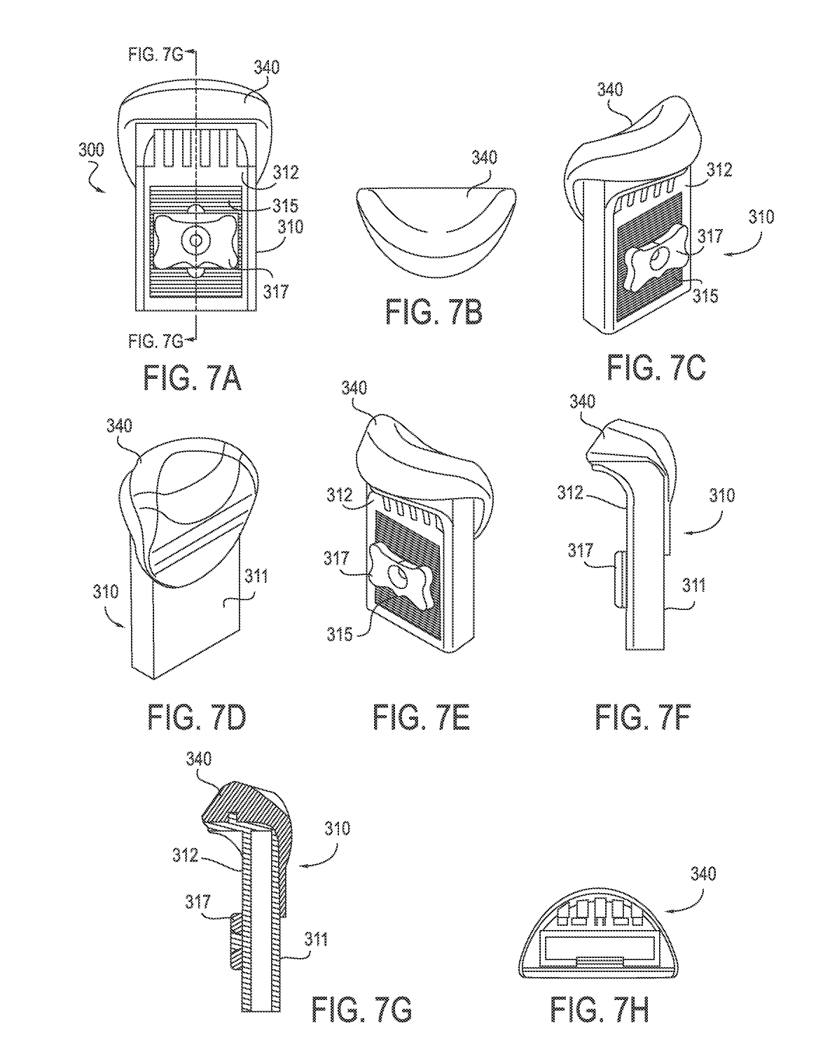

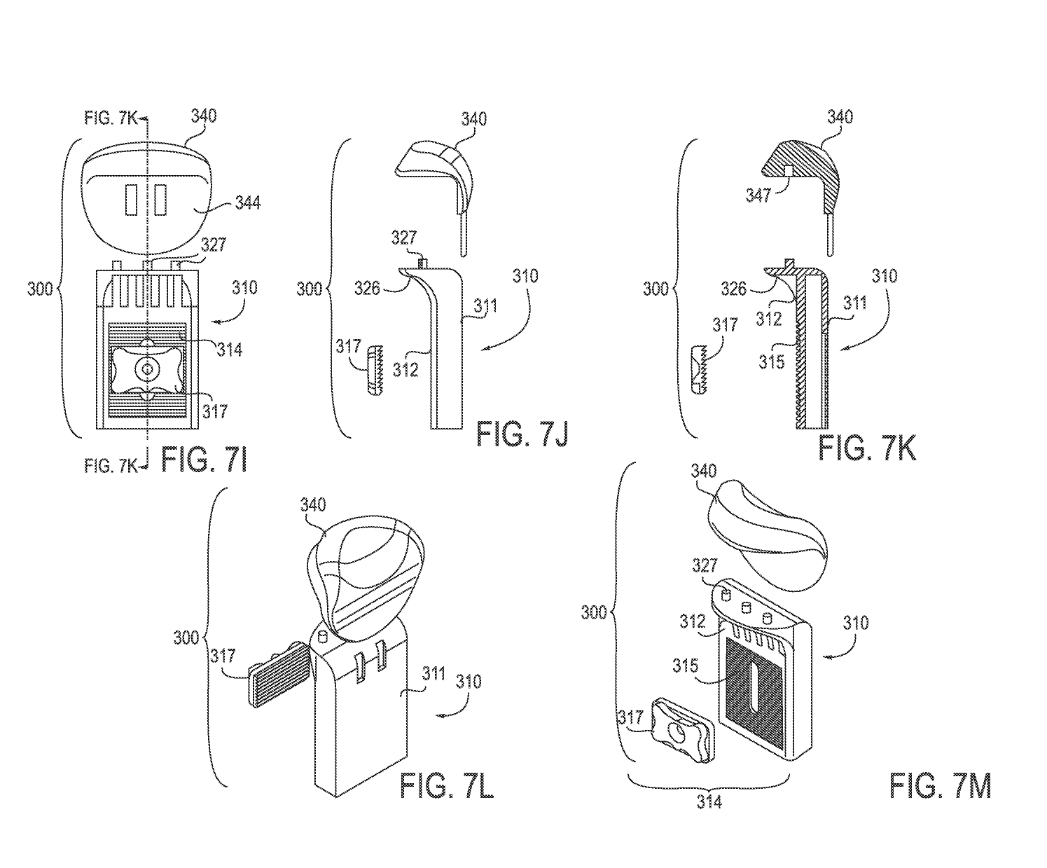

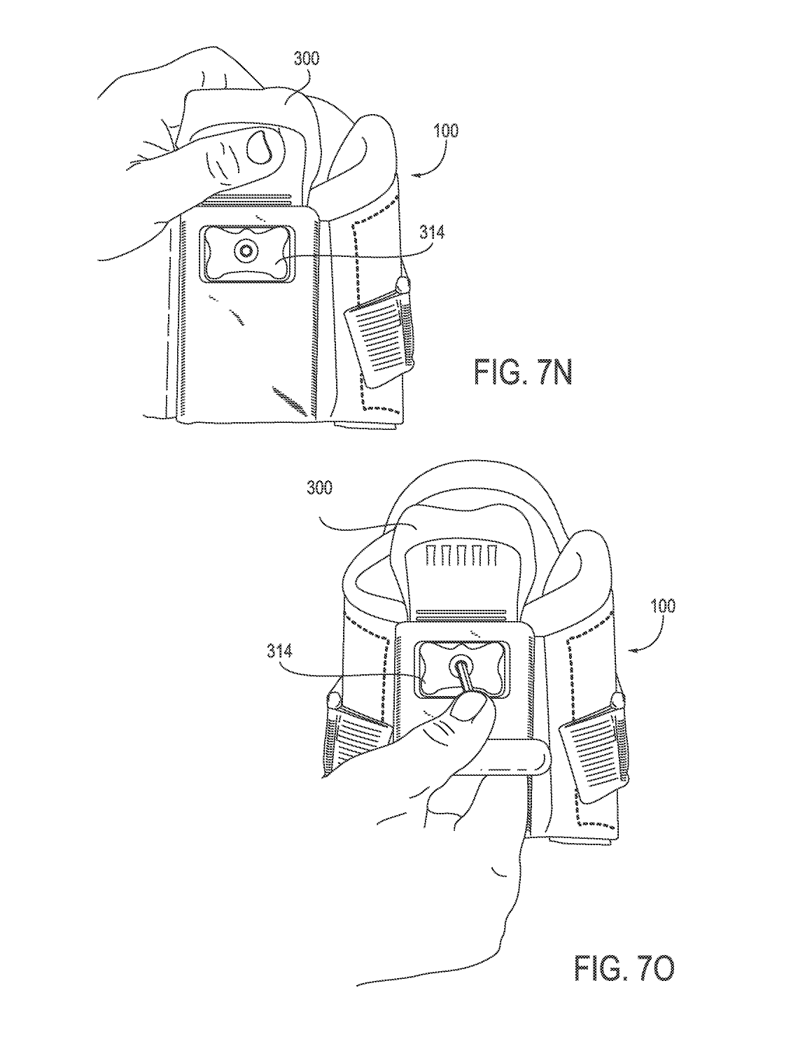

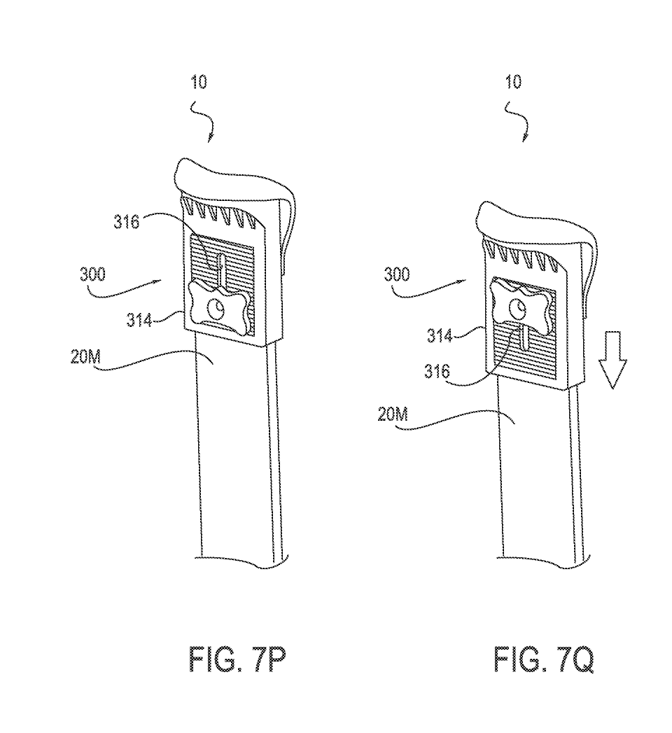

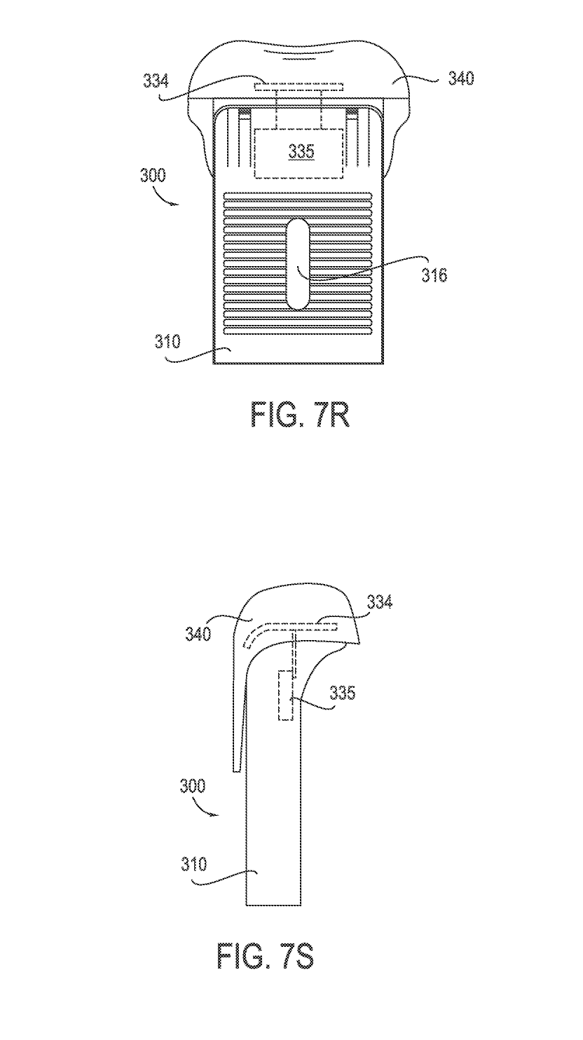

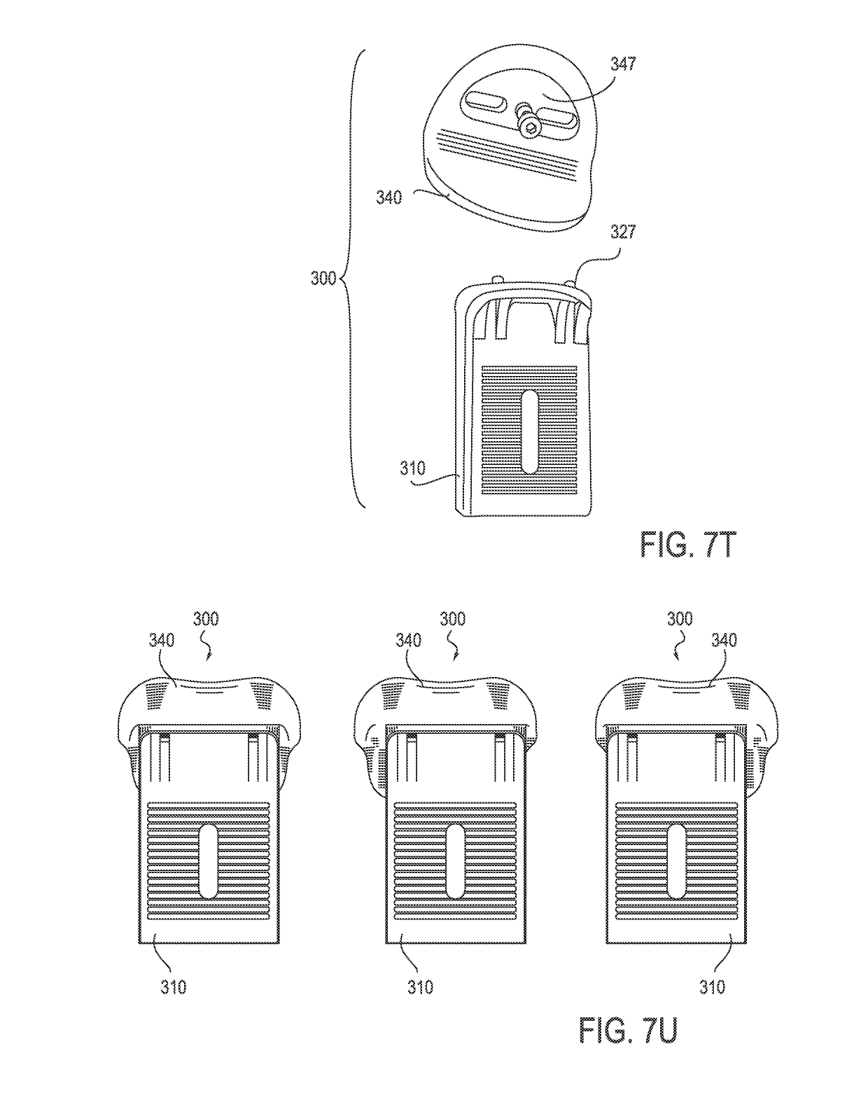

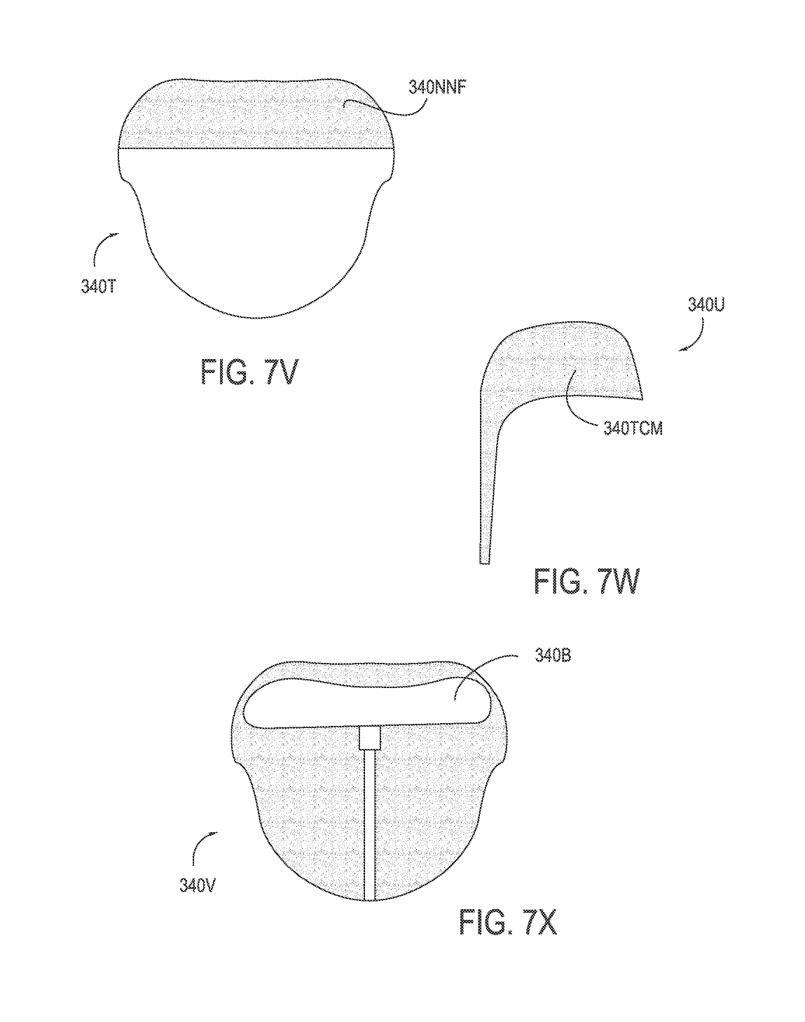

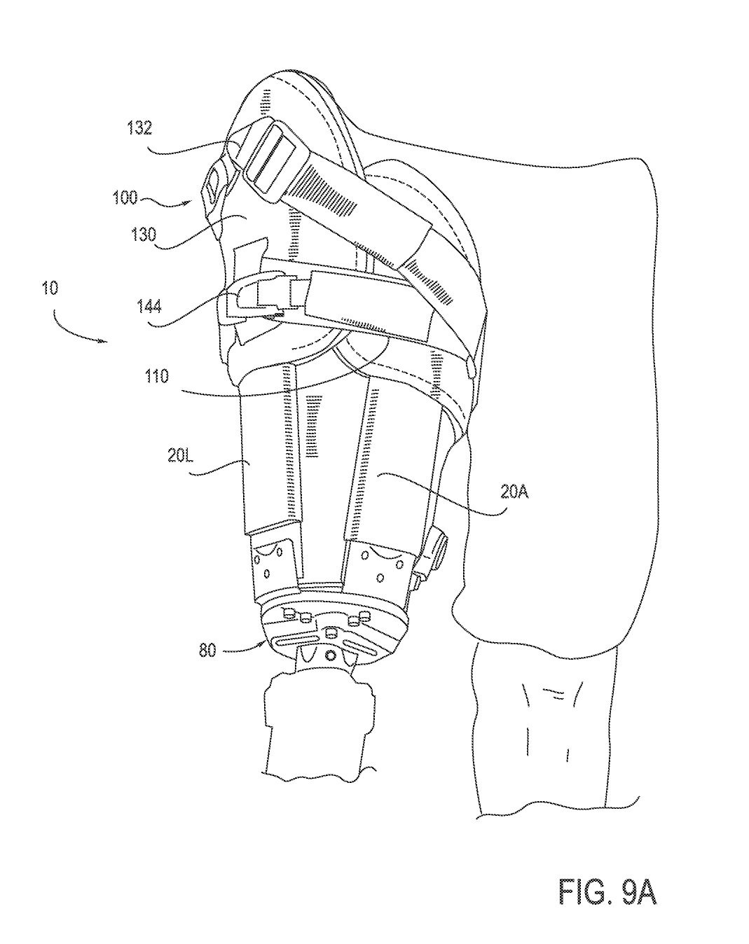

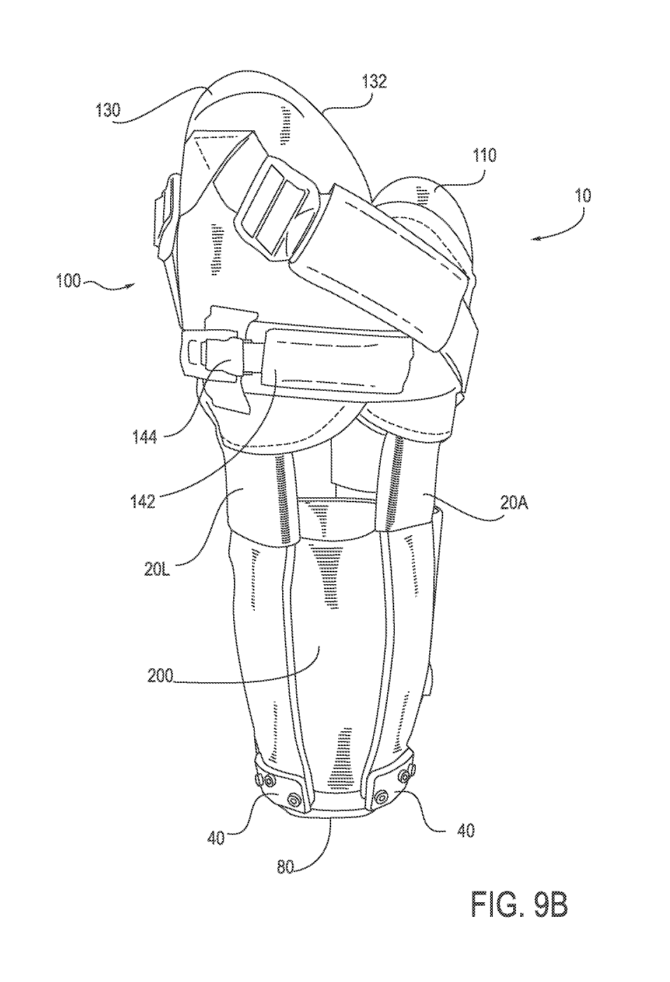

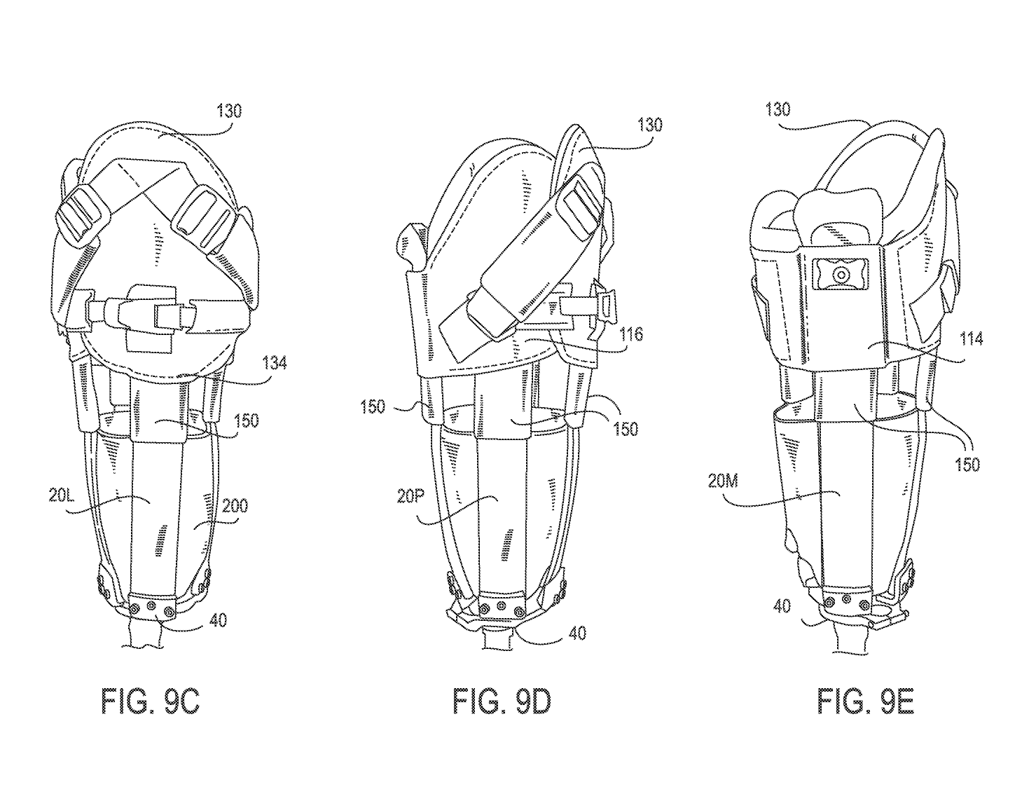

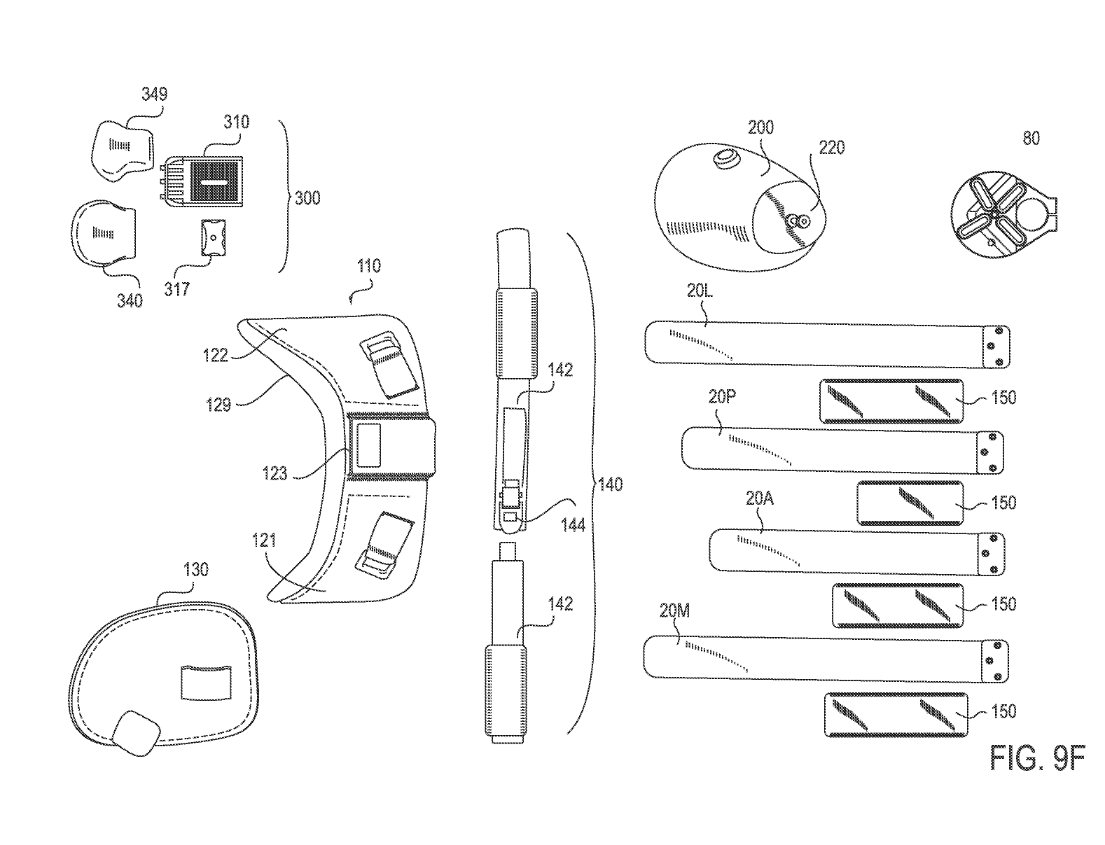

Referring now to FIGS. 4A, 4B and 9A-9F, thermoplastic fiber composite struts will be generally designated herein as struts 20. Typical embodiments of a modular prosthetic socket 10 include four struts, although embodiments may include fewer or more than four. In the typical example of a modular prosthetic socket 10 with four struts, the struts may be identified by their relative circumferential position with respect to the patient's residual limb: medial strut 20M, anterior strut 20A, lateral strut 20L, and posterior lateral strut 20P. The absolute circumferential position of these struts, in a clockwise/counter-clockwise orientation differs according to whether modular prosthetic socket 10 is assembled and configured for a right leg or a left leg. For simplicity, a modular prosthetic socket 10 arranged for the right leg of a patient will be the general configuration described and depicted herein. Positioned on the proximal end of strut 20M is an adjustable height (or height adjustable) ischial seat assembly 300, which is described in further detail below, as well as being depicted in FIGS. 7A-7O. In addition to multiple longitudinal struts 20, modular prosthetic socket 10 embodiments further may further include a distal base 80, anchoring insert 220, and adjustable height ischial seat assembly 300. Struts 20 and distal base 80 may be collectively referred to a prosthetic socket frame.

A circumferential position-specific arrangement of struts 20 is shown in FIGS. 4A and 4B. Embodiments of medial strut 20M may be alternatively referred to as the “ischial strut”, because its position within the socket places it below the ischium of the patient. The medial strut 20M is generally the largest strut, and the one that most significantly bears weight (see section below that addresses the adjustable medial strut cap). Further, medial strut 20M is generally rigged into a brim embodiment first when assembling a modular prosthetic socket 10, and of all the struts, can be considered the anchoring strut. Further still, inasmuch as adjustable height ischial seat assembly 300 is mounted on medial or ischial strut 20M, this particular strut, and its proximal end in particular, may also be referred to an adjustable height ischial seat assembly mounting site.

Embodiments of the individual thermoplastic fiber composite struts 20 may all be identical in dimension and composition, but typical embodiments can vary in dimension from each other. They may also vary in terms of composition, but in typical embodiments of the modular prosthetic sockets 10, the composition of included struts 20 is the consistent. In typical embodiments of a group of struts included within a modular prosthetic socket 10, the medial strut 20M is thicker than its three cohorts. In one example of how struts 20 can vary among each other, medial strut 20M can be used as a reference. Medial strut 20M is an 8-ply thermoplastic fiber composite structure, while the other struts (anterior strut 20A, lateral strut 20L, and posterior strut 20P) are all 6-ply structures. In typical embodiments of a modular prosthetic socket 10, the absolute strut length can vary, in a custom-manner, according to the length of the patient's residual limb. However, using the medial strut 20M as a reference “zero” point, in one example, the anterior strut 20A is −25 mm, the lateral strut 20L is +50 mm, and the posterior strut 20P is +25 mm.

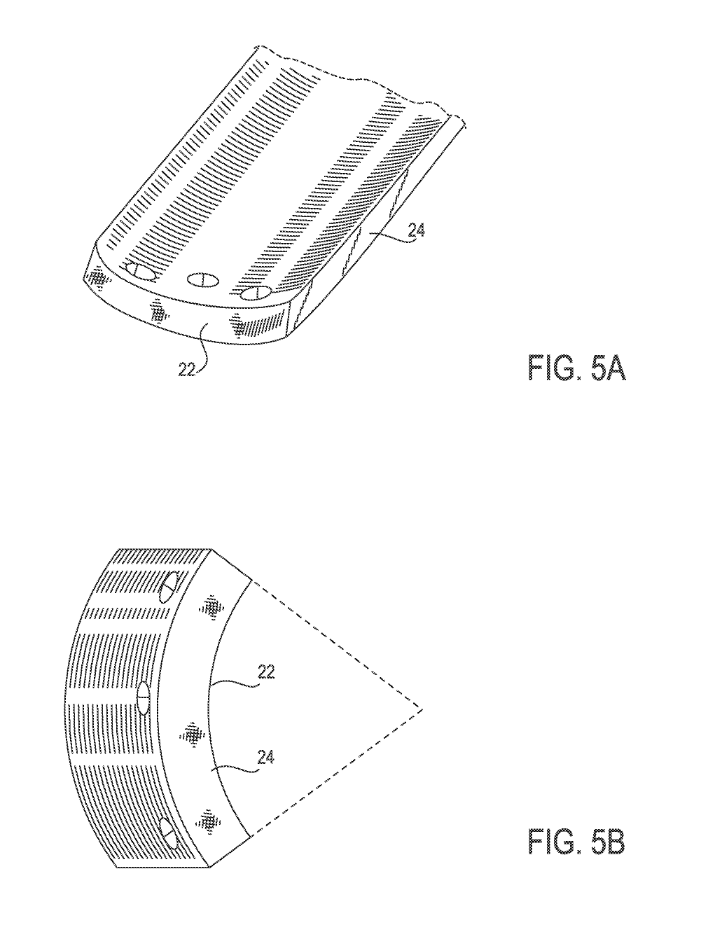

Embodiments of thermoplastic fiber composite struts, as described in U.S. patent application Ser. No. 14/213,788, and as depicted in FIGS. 5A-7D therein, are all long flat pieces that have no particular baseline deviation from being flat at their distal end. These embodiments can be thermally reformed to assume innumerable desired shapes, but the initially formed embodiment has a flat distal end. In a retroactive characterization, and with reference to the flat distal end, the strut embodiments of U.S. patent application Ser. No. 14/213,788 may all be considered Type A strut embodiments. The present application introduces Type B strut embodiments, which differ from Type A at least by having contoured distal aspects.

In some thermoplastic fiber composite strut embodiments, the distal end of strut includes a longitudinally aligned inward facing concavity. This contoured feature allows strut to sit well on the strut ledge 48 of a strut connector base portion 41, abutting securely against back portion 60 of a strut connector 40. This relationship is detailed further in the context of the included description of strut connector embodiments.

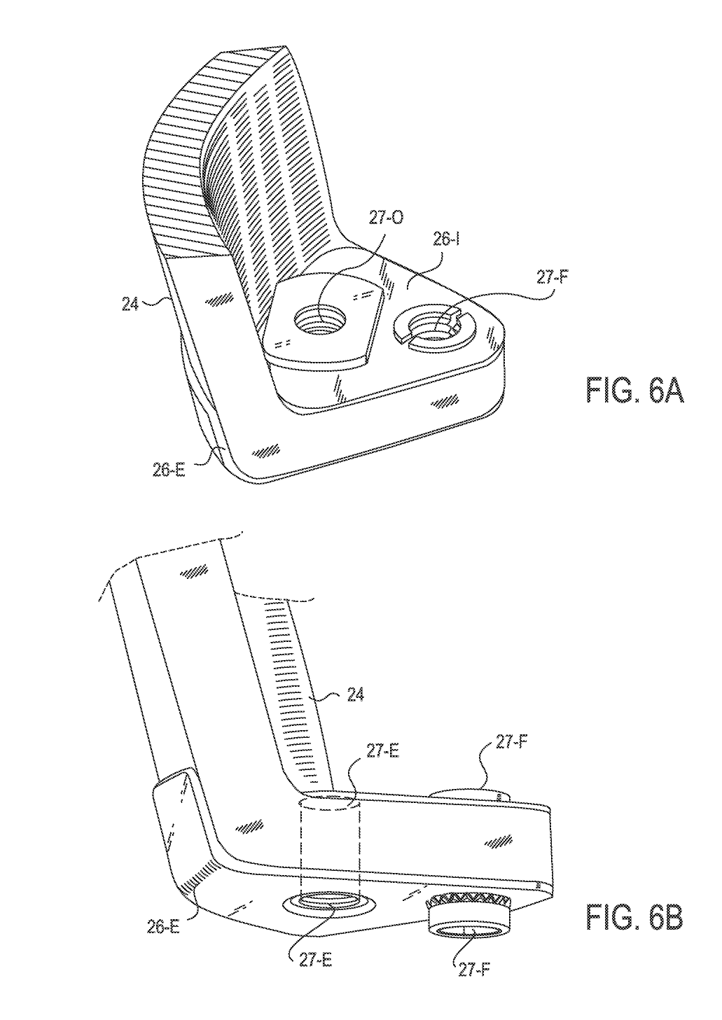

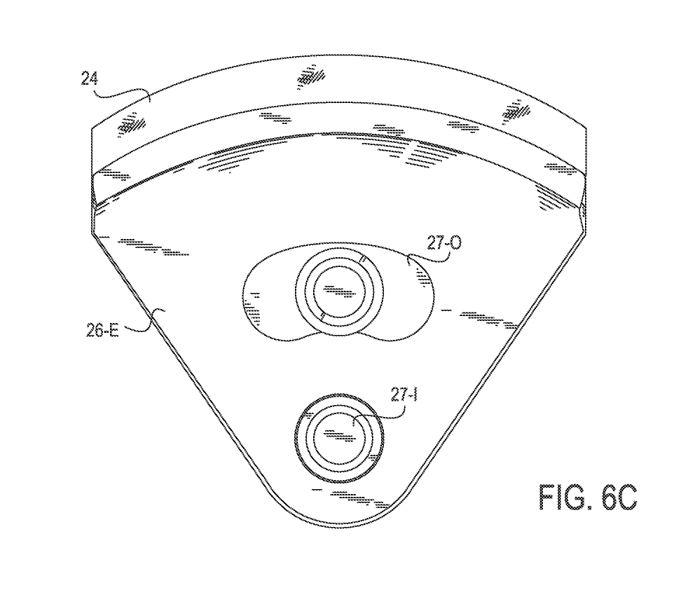

This structural concavity may be added to strut after a primary molding that yields a substantially flat strut, such as those depicted in U.S. patent application Ser. No. 14/213,788 (FIG. 6 therein); inward facing concavity may be added to a flat strut by way of a secondary thermal reforming process. In other embodiments the primary strut mold can be configured to provide the inward facing concavity. In addition to providing an efficient and well-supported fit between a strut and a strut connector 40, this feature strengthens the distal portion of strut by creating a second moment of area that particularly resists an externally directed force from within the socket.

### Struts and Strut Connectors

Referring now to FIGS. 5A, 5B and 6A-6C, another embodiment of a strut 24 has a distal end with an inward facing concavity, but which further has a centrally bent distal end. The centrally bent distal end may further include a metal cladding 26-I disposed on its internal surface and a metal cladding 26-E disposed on its external surface. Metal cladding elements 26-I, 26-E, together, may be considered elements ancillary to strut 24, or alternatively, they may be considered as a type of strut connector, functionally analogous to strut connector 40.

There are both similarities and differences with respect to the arrangement represented by strut connector 40: for example, strut embodiment 24 does not make use of a pivot-securing tab 50. Strut embodiment 24 does have a similar arrangement of inner 27-I and outer 27-O bolts that connect through strut slots 84 of distal base 80, and allow pivoting of the strut around the inner bolt, as does strut connector 40. Inner and outer, as applied to bolts or bolt holes refers to relative position on a radial line. Inner 27-I and outer 27-O bolt holes penetrate though both internal 26-I metal cladding surfaces and external metal cladding surface 26-E, disposed as they are on either side of strut 24. Outer bolt hole 27-O is kidney shaped; this configuration allows the outer portion of the base portion of the strut 24 to pivot around the fixed site represented by the inner bolt hole 27-I.

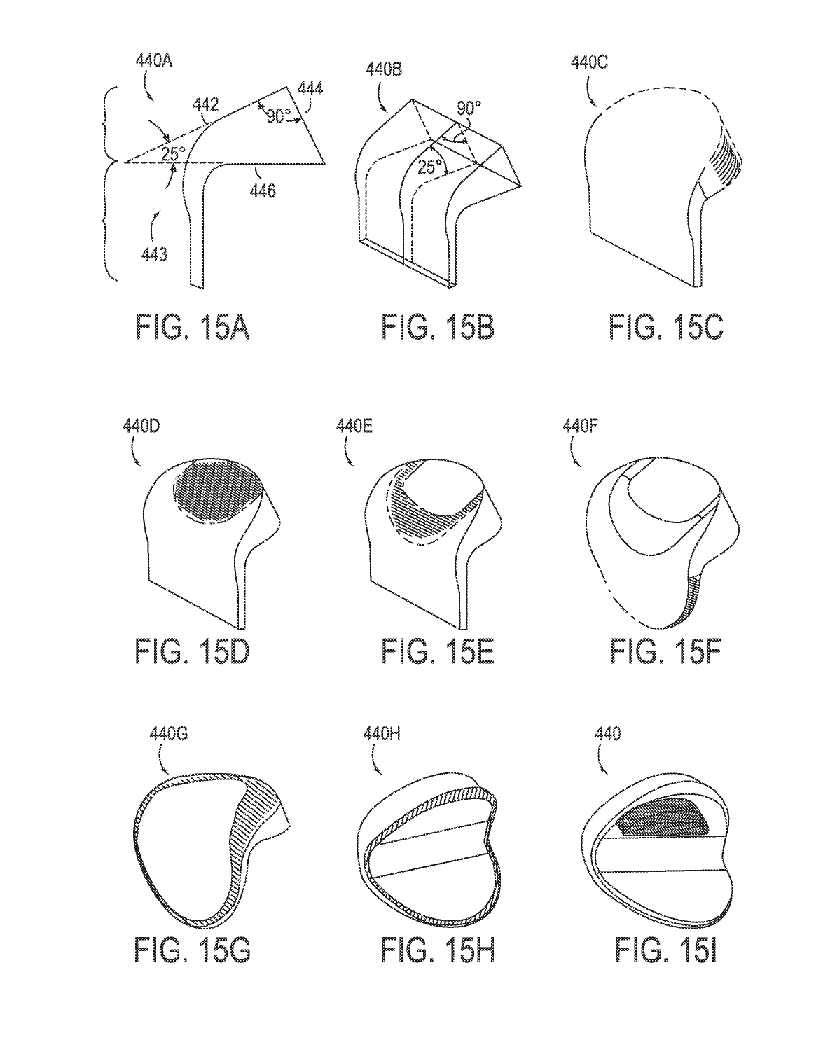

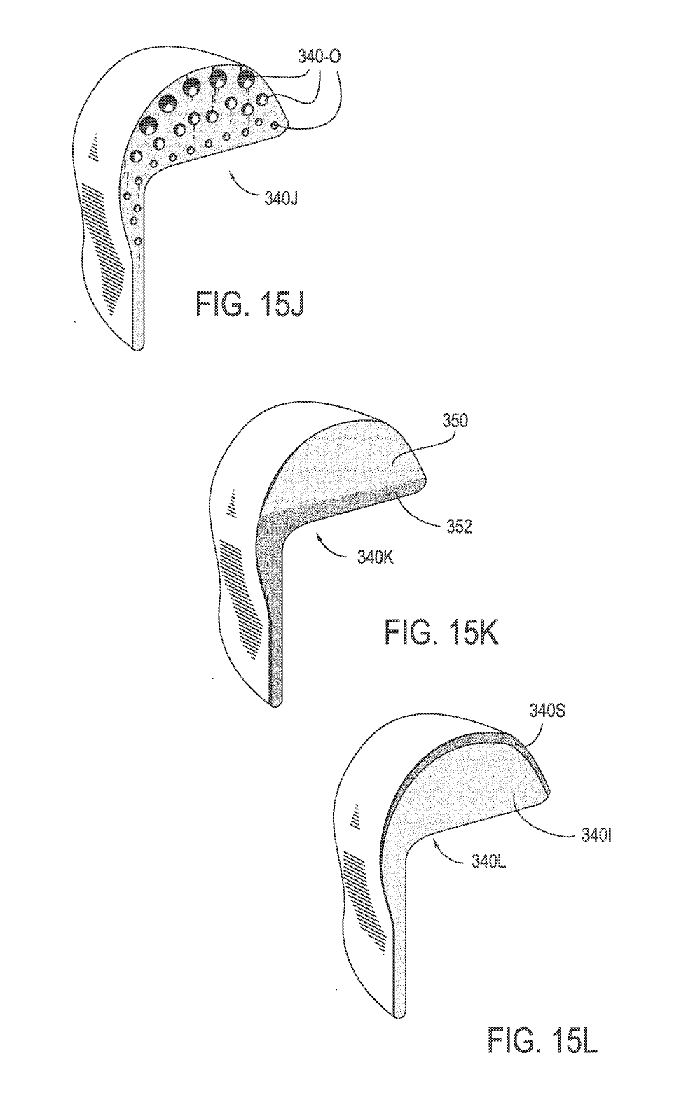

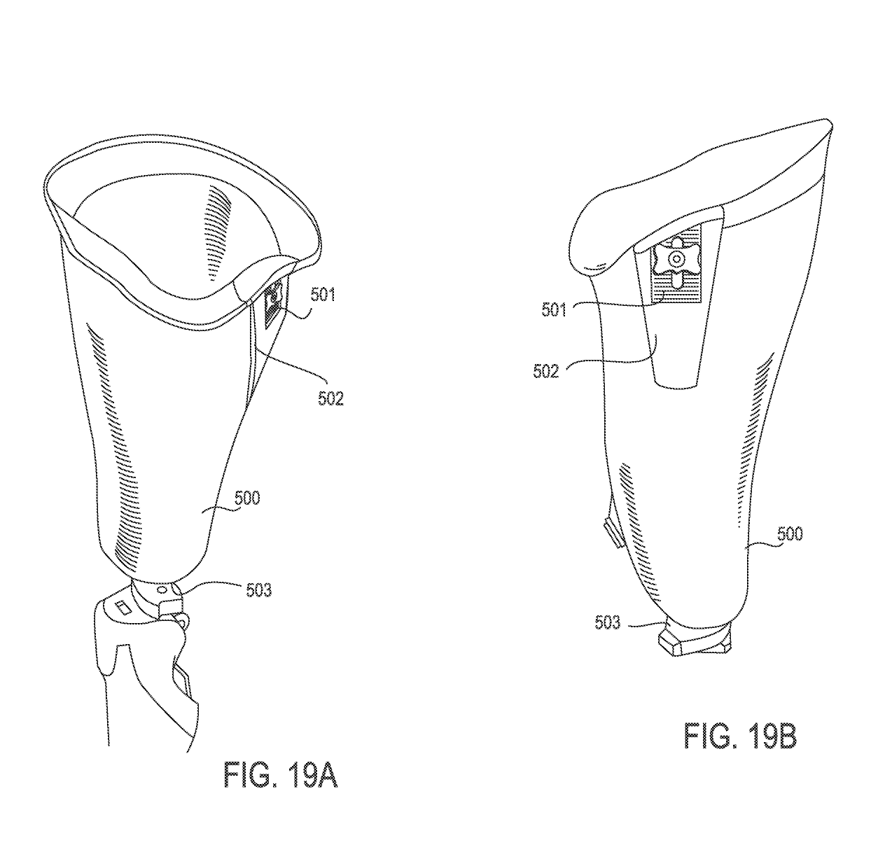

It is noteworthy that the distal end of strut 24, when assembled to a distal base 80, provides a distal facing flat surface that engages the proximal flat surface 81 of the distal base, the engagement between the two surfaces is direct, without any mediating element, and the pivoting movement that strut 24 is able to perform is not constrained by any feature on the surface of the distal base. Locking of pivotal movement is effected only by frictional engagement of these apposed flat surfaces. This arrangement on a distal base contrasts, for example, with embodiments of a distal base as described in U.S. patent application Ser. No. 14/213,788, where indented portions of proximal plate of a distal base limit pivoting movement of a strut connector (see FIGS. 15G, 19A, 19B therein).

As disclosed in U.S. patent application Ser. No. 14/213,788, in some embodiments of a longitudinal strut made of a thermoplastic-fiber composite material, the material may include or consist of polymethylmethacrylate (PMMA) polymer with carbon fibers embedded therein. Such material typically is formed commercially as sheets, with such sheets including carbon fiber in continuous or substantially continuous form. In forming thermoplastic fiber composite strut embodiments, as disclosed herein, the material is typically arranged in multiple layers or plies.

In some particular embodiments, the carbon fibers are arranged with a 12K tow, referring to bundles of continuous fiber with about 12,000 fibers per bundle. The bundles are arranged as a twill weave, the weave having two bundle populations oriented perpendicular to each other. Within the context of the strut, the woven bundles are oriented such that one population is parallel to the longitudinal axis of the strut, and the other is orthogonal to the longitudinal axis of the strut. The thickness of each ply is in the range of about 0.8 mm to about 0.9 mm. Thus, the thickness of a 6-ply strut is about 5 mm, and the thickness of an 8-ply strut is about 7 mm.

Typical embodiments of struts are non-cylindrical in cross section; their width is greater than their depth. The cross sectional shapes may be flattened and rectangular, they may include curvature on either or both of the internal and external surfaces, they may be symmetrical or asymmetrical with respect to their internal and external surfaces, and they may be oval in cross section. By way of examples, in some embodiments, the width of a strut can range between about 2 cm and about 6 cm. In particular embodiments, the width of a strut can range between about 4 cm and about 5 cm.

There are several functional advantages of this rectangular or oval shape that are related to directional biases in flexibility. For example, a relatively wide cross sectional profile provides a wide surface across which to distribute pressure. In another functional example, it is advantageous for the struts to be able to flex, or to be reformable in a plane that includes the axis parallel to the width of the strut. The freedom to be able to flex in this dimension allows customization of the cross sectional profile (or volume) of the prosthetic socket to accommodate individual variation among patients, and allows, by way of reformability of thermoplastic-fiber composite materials, to make adjustments in these dimensions. On the other hand, it is advantageous to disallow flexing, or to discourage thermal reformability within the plane that includes the longitudinal axis of the strut (a “sideways” flexing). Such flexing would create weakness in the longitudinal aspect of the strut-based prosthetic socket structure. Adjustability in the circumferential position of struts is advantageous, but this capability is readily handled by adjustments in the strut connectors positioning on the distal base, as described elsewhere.

In addition to the advantageous flexibility bias imparted by the flattened or rectangular cross sectional profile of thermoplastic fiber composite struts, per embodiments of modular prosthetic socket 10, the orientation of fiber layers or plies in the thermoplastic plastic can be significant. For example, per embodiments provided herein, the fibers are typically oriented either parallel to- or orthogonal to the longitudinal axis of the strut.

It is advantageous for the materials that form the strut have a high strength/weight value. Additionally, it is advantageous, particularly during the fabrication process, for the material to be sufficiently formable to conform to a desired contour profile, and for such formability to not adversely affect the structural integrity of the strut. Other material properties that are generally advantageous for this technical application include a high elastic modulus. Accordingly, thermoplastic fiber composite materials, as described above, particularly by the example provided, are highly suitable.

In alternative embodiments, however, modular prosthetic socket components may include other materials and methods of fabrication that are different from those used to form and reform thermoplastic fiber composite materials. The fundamental features of the technology that remain in common are described in U.S. patent application Ser. Nos. 13/675,761 and 14/213,788. Several attributes will be now be recited. These include a prosthetic socket that has a structure that is strut-based and modular. Typically, the struts are integrally independent, circumferentially non-contiguous at the proximal end. Further still, an arrangement of strut connectors disposed on a distal base plate provide for a high degree of volume adjustability. As noted in U.S. patent application Ser. No. 13/675,761 (paragraph 128), under some circumstances, materials such as aluminum, fiberglass, bamboo, and locally available thermoset resins may be suitable.

The formability and repeatable reformability of thermoplastic-fiber composites is practical in a manufacturing sense since it allows a relatively small number of forms in an inventory to be amplified into a large number of desirable forms by thermal reforming. However, 3D printing offers an alternative approach to creating a large number of desired forms directly, without having to go through a common physical embodiment prior to being shaped into final desired form.

As described above, embodiments of a modular prosthetic socket 10 include a medial or ischial strut 20M, so named (medial) because of the circumferential position within the socket that this strut occupies, as well as because of its anatomical position, which places it below the ischium of the patient. This strut 20M, in some embodiments, is more substantial (e.g., wider, thicker) than the other struts. Medial strut 20M is functionally significant in that it is positioned and configured to bear weight brought down on it through the ischium. Of the various bones within the pelvis, the ischium is particularly adapted to bearing weight when a human is in a sitting position. The structure of modular prosthetic socket 10 takes advantage of the natural role and structure of the ischium, recruiting it to bear patient weight when the patient is standing or active while wearing the prosthetic socket.

### An Adjustable Height Ischial Strut Cap Assembly

To facilitate this weight bearing role, embodiments of modular prosthetic socket assembly 10 may include an ischial (or medial) strut cap assembly 300, which fits over the proximal end of medial or ischial strut 20M, is configured to bear weight, and has an adjustable height by way of a telescoping mechanism. Aspects and embodiments of ischial strut cap assembly 300 are shown in FIGS. 7A-7O. FIGS. 7A-7M are rear (7A), top (7B), rear perspective (7C), front perspective (7D), rear perspective (7E), side (7F), cross-sectional side (7G), cross-sectional top (7H), rear exploded (7I), side exploded (7J), cross-sectional side exploded (7K), front perspective exploded (7L), and rear perspective exploded (7M), views, respectively, of adjustable ischial strut cap assembly 300, according to one embodiment. FIGS. 7N and 7O are both rear perspective views of a modular prosthetic socket, including adjustable ischial strut cap assembly 300, showing a user loosening the adjustable height locking mechanism, which allows strut cap assembly 300 and a seat pad thereof to telescopically adjust up or down, according to one embodiment.

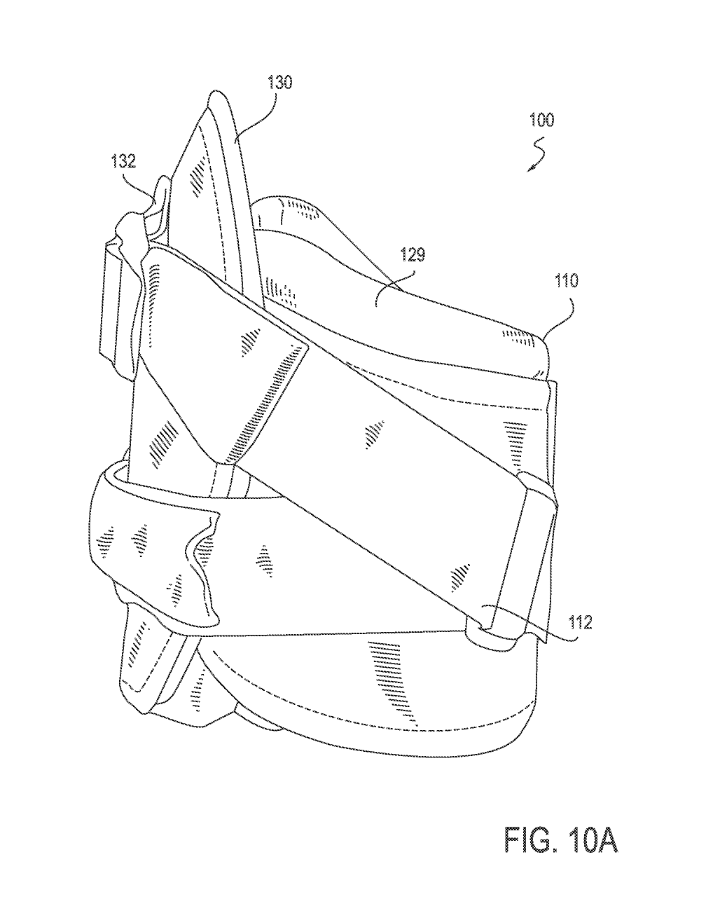

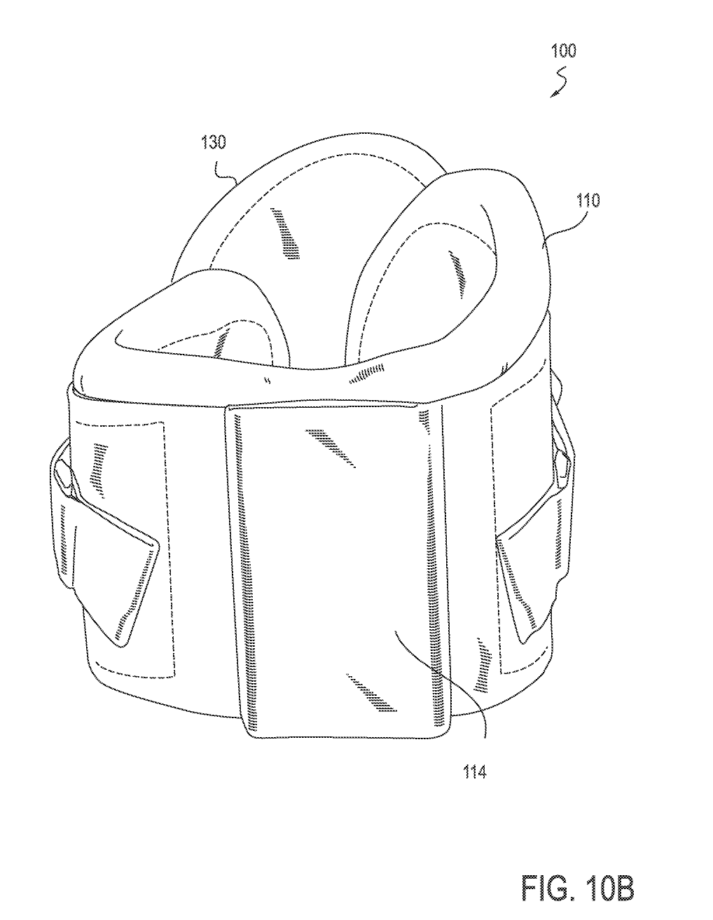

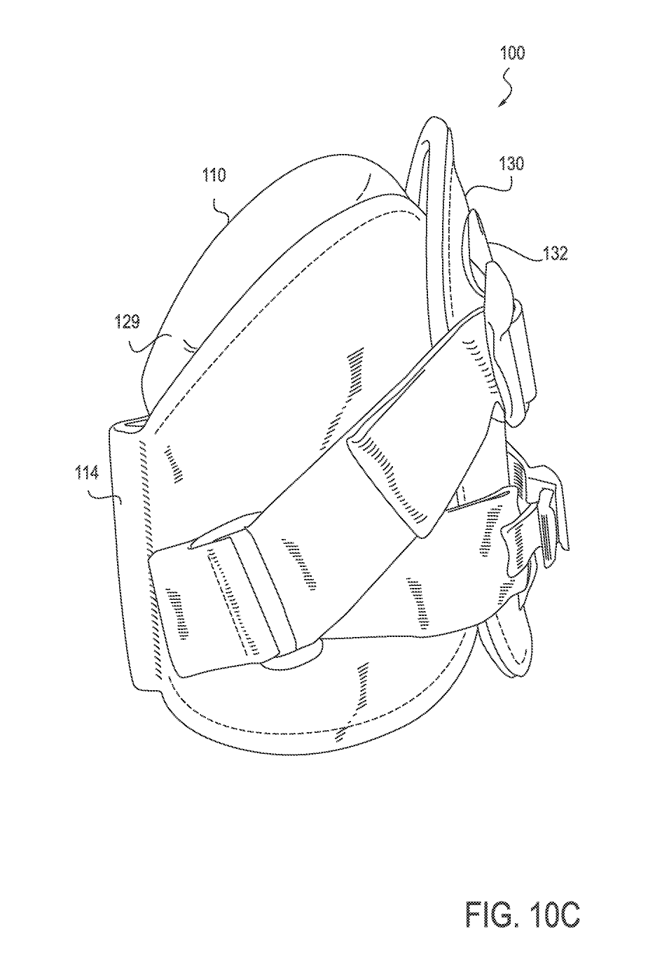

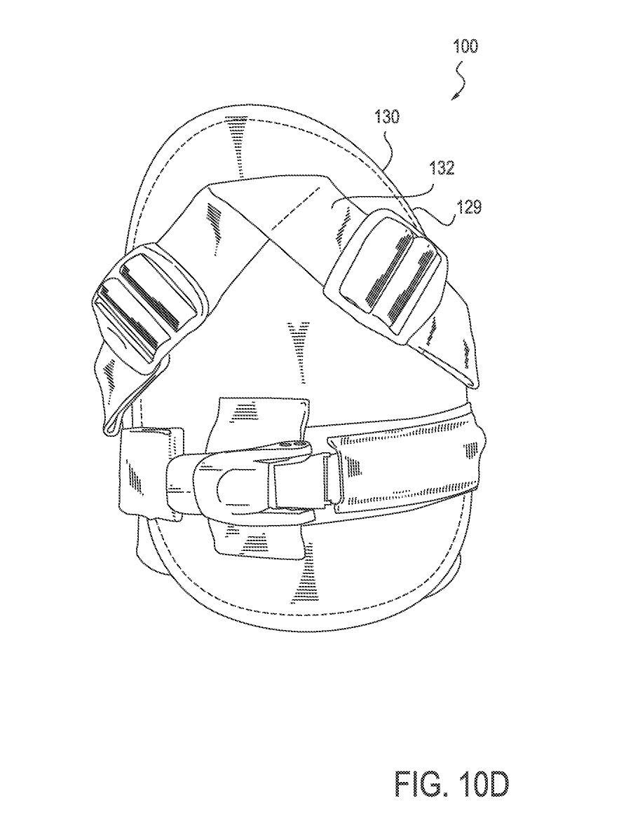

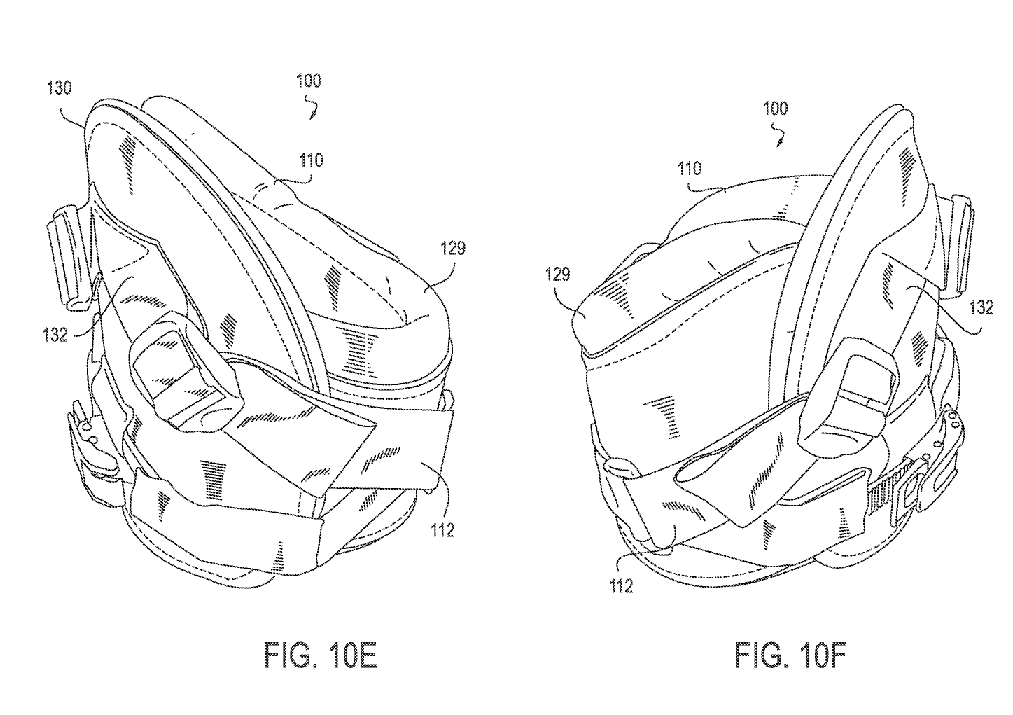

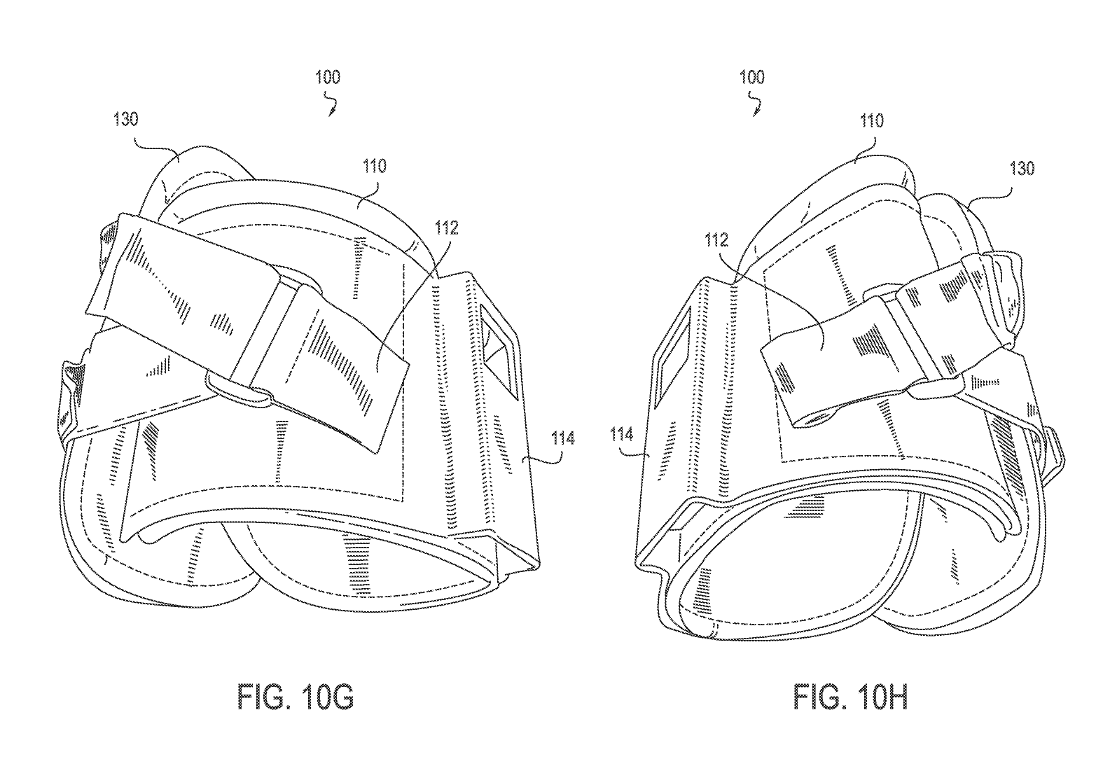

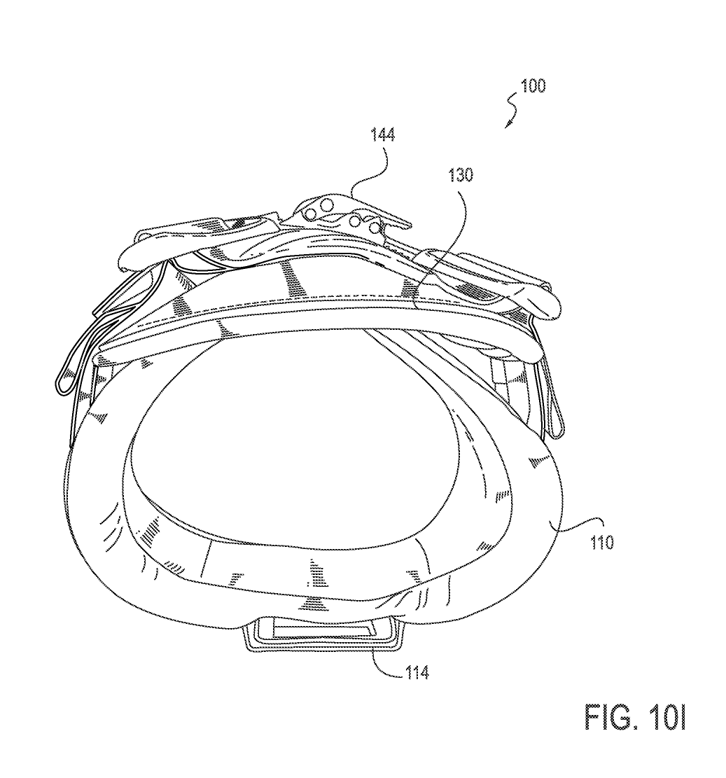

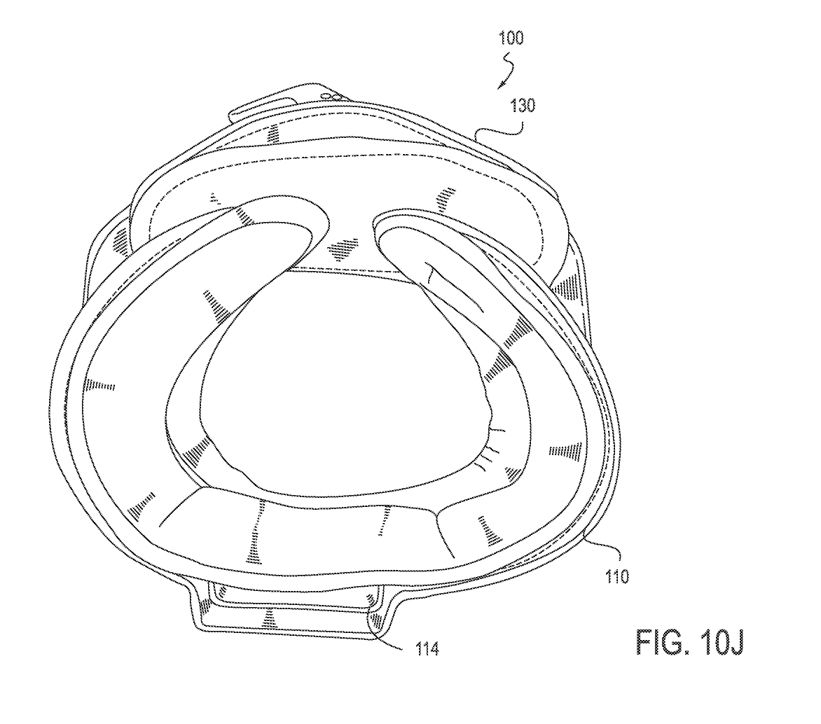

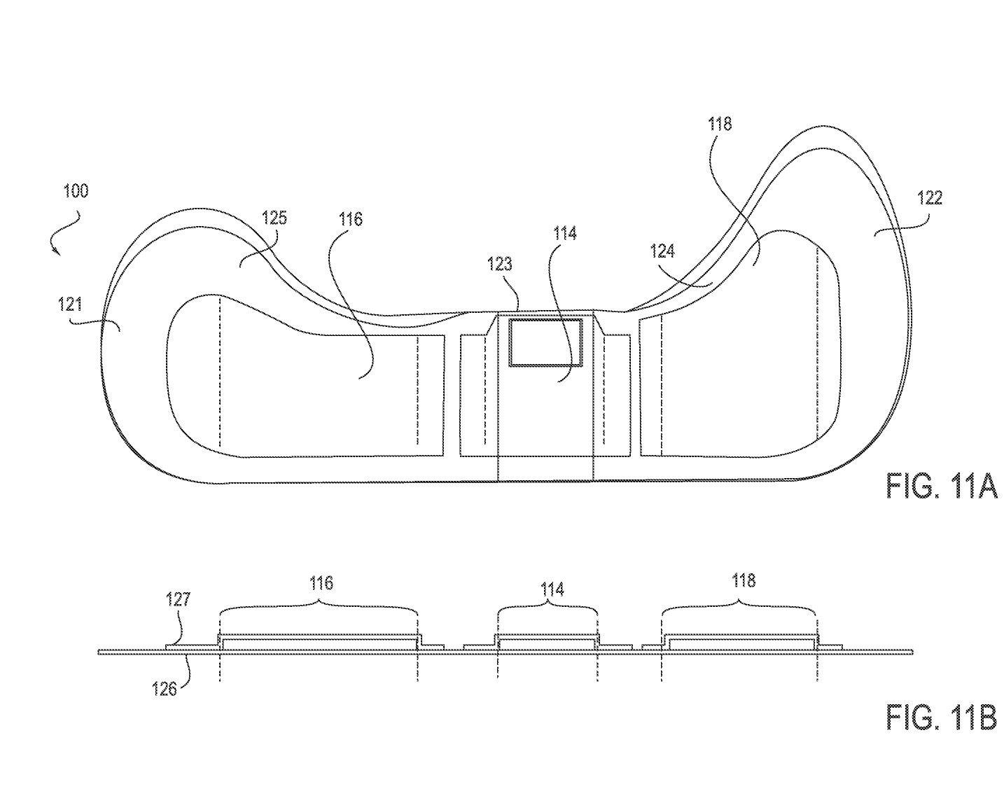

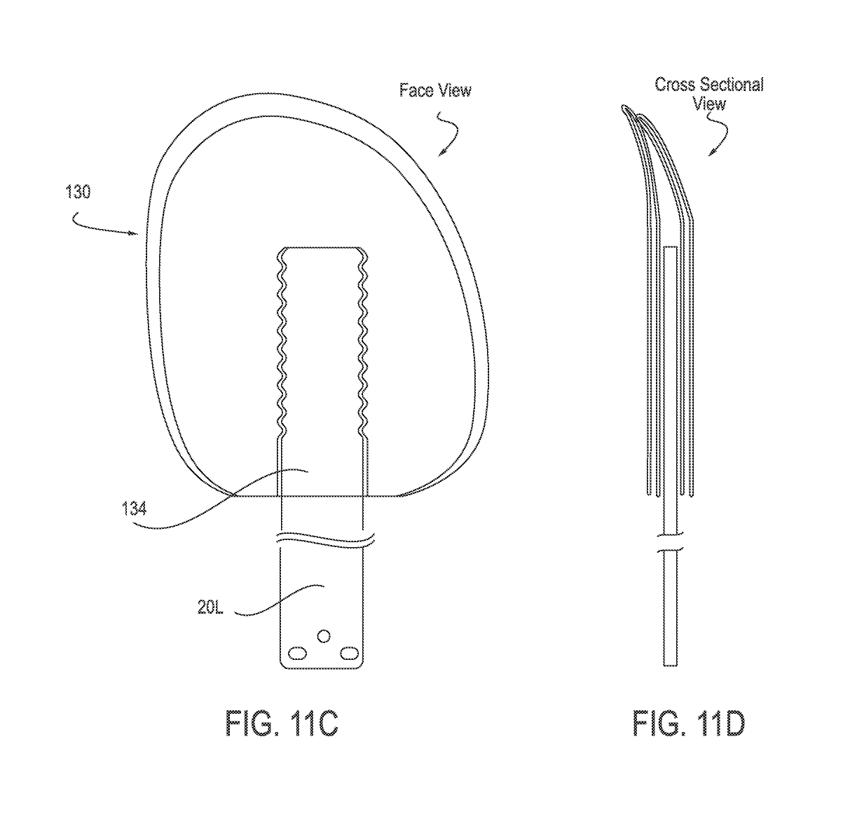

The telescoping height adjustability associated with ischial strut cap assembly 300 is in relation to the socket as a whole, but most particularly to the distance between the proximal end of the strut and distal base 80, to which medial strut 20M is attached at its distal end. To place medial strut 20M and ischial strut cap assembly 300 in a broader context, the portion of strut 20M immediately distal to the site of the strut cap assembly 300 is disposed within an ischial or medial strut channel 114 in the medial section 123 of butterfly wrap segment 110 of brim 100, as described below and shown in FIGS. 10A-11D.

Referring now to FIGS. 7A-7O, one embodiment of ischial strut cap assembly 300 will be shown and described in more detail. In general, ischial strut cap assembly 300 may be disposed over the distal end of ischial strut 20M (not shown in these figures), to facilitate weight bearing by, and comfort relative to, ischial strut 20M. In the illustrated embodiment, ischial strut cap assembly 300 includes an ischial seat pad 340 and a strut cap base 310 (or another form of an ischial seat pad mounting member in alternative embodiments). Strut cap base 310 helps support ischial seat pad 340 in its ischium-proximate position atop a prosthetic socket strut and on a prosthetic socket itself, such as modular prosthetic socket 10, or a laminated socket 500, as shown in FIGS. 19A-19B, as described further below. Strut cap base 310 may include a height locking mechanism 314, a locking square 315, and a height locking plate 317, all of which may all be referred to, collectively, as a prosthetic socket frame mounting mechanism, by which ischial seat pad 340 is adjustably and lockably mounted onto ischial strut 20M. Strut cap base 310 has a rectangular box like structure with a closed proximal end 325 and an open distal end 328 that can fit over an inserted proximal end of ischial strut 20M. Strut cap base 310 also includes an internal face 311 and an external face 312. Internal and external, in this context, refer to positioning in accordance with internal and external aspects of ischial strut 20M and with respect to modular prosthetic socket 10 as a whole. FIGS. 7N and 7O illustrate ischial strut cap assembly 300 in place in modular prosthetic socket 10, with a user adjusting the former.

In some embodiments, ischial strut cap assembly 300 includes an adjustable telescopic height locking mechanism 314, which allows the height of strut seat or pad 340 to be secured at a desired height or elevation above distal base 80. FIGS. 7P and 7Q show a distal end of a medial or ischial strut 20M with an adjustable height strut cap assembly 300 mounted distally thereon in two positions (brim 100, as shown in preceding FIGS. 7N and 7O are not shown in this view, for clarity). FIG. 7P shows adjustable height strut cap assembly 300 in its highest position relative to strut 20M (and highest relative position with respect to distal base 80, not shown). By manipulation of locking mechanism 314 (as seen in FIGS. 7N and 7O, in particular), adjustable height ischial strut cap assembly 300 can be dropped to a minimal height, as seen in FIG. 7Q.

In one embodiment of a locking mechanism 314 comprises a friction-based locking mechanism wherein a locking plate can be releasably pressed or secured against a surface of strut cap base 310; in the absence of friction, strut cap base 310 can move vertically relative to medial strut 20M; when friction is applied, strut cap base 310 is locked into a fixed position on medial strut 20M. More specifically, external face 315 of strut cap base 310 includes a locking square surface feature 315 that includes a horizontally ridged surface and a longitudinally aligned slot 316. A height locking plate 317 with an internal surface having horizontal ridges complementary to those of locking square 315 has a locking element 318 that reaches through slot 316 and a releasable default configuration that pulls locking square 315 and height locking plate 317 together in a manner that engages their complementary ridges, thus locking them together, preventing vertical movement. When the height locking plate 317 is manually pulled by a user, the complementary ridged surfaces of locking square 315 and locking plate 317 disengage, and a user can adjust the height of strut cap base 310 upward or downward on the distal end of strut 20M. By adjustment of locking mechanism 314, a patient can optimize the elevation of ischial strut cap assembly 300 so that ischial seat pad 340 most effectively engages the ischial tuberosity, and most effectively allows distribution of body weight load through the ischial tuberosity and away from the alternative path wherein body weight load is transferred through the distal end of the residual limb.

In some embodiments of strut cap base 310, the proximal end or surface 325 includes an externally projecting lip 326 as well as positioning elements 327 that are complementary to positioning receptacle or elements 347 on the distal or bottom surface 346 or seat pad 340. As ischial strut cap assembly 300 is assembled, ischial seat or pad 340 fits over the proximal end 325 of strut cap base 310; it extends over externally projecting lip 326, and is stabilized in position by the positioning elements noted above.

### Optional Features of an Ischial Strut Cap Assembly

In some embodiments, as illustrated in FIGS. 7R-7S, ischial strut cap assembly 300 may also include a force sensor and microprocessor and transmitter unit. The present application claims priority to U.S. Provisional Patent Application Ser. No. 62/161,132, filed May 13, 2015, which is directed toward a prosthetic socket that is sensor enabled to provide for clinical use and mechanical adjustments, and which is incorporated into the present application, with particular reference to FIG. 4 of that application and associated description. FIGS. 7R and 7S show, respectively, a transparent external side face view and a transparent side view of height-adjustable strut cap assembly 300, including a force resistive sensor 334 disposed within ischial seat pad 340 and operatively connected to a microprocessor-transmitter assembly 335.

The interface represented by ischial seat pad 340 and strut cap assembly 310 connection is one across which body weight load is transmitted. The load, as a whole, when body weight is on that limb, is distributed between transmission through (a) the ischial tuberosity and (b) the distal end of the residual limb. This distribution of load between these two alternative load paths is may be pertinent data for studies of groups of patients and/or for the individual patient. In general, it is beneficial for the patient to transmit a significant percent of the load through the ischium, sparing the distal end of the residual limb from having to bear the bulk of body weight load.

In various embodiments of the invention, microprocessor-transmitter assembly 335 transmits data to receivers either on another site on a prosthesis, a receiver being worn by the patient, and/or to a network external to the patient or an article worn by the patient. Per further embodiments of the invention, these force resistive sensing data may be collected and analyzed, or they may be directed to automated and appropriate responses by way of actuators within the larger prosthesis or within the prosthetic socket. Either by way of automated or by manual adjustments in response to force data derived from force resistive sensor 334, the level of force transmitted through the ischium may be adjusted. Various load distribution adjustments may include adjusting the height of adjustable height strut cap assembly 300 (FIGS. 7N, 7O), adjusting the horizontal position of ischial seat pad 340 with respect to strut cap base 310 (see description of FIGS. 7T-7U, below), and/or by adjusting the struts, or brims, or other components of the hosting prosthetic socket.

FIGS. 7Y-7U show an alternative embodiment of an ischial strut cap assembly 300, in which ischial seat 340 is horizontally slidable with respect to the ischial strut cap base 310 supporting it. FIG. 7T shows an embodiment of ischial seat pad 340 and a strut cap base 310 disconnected from each other in order to expose details of receptacle 347 that slidably mate with positioning features 327 of strut cap base 310. This arrangement permits ischial seat pad 340 to travel left and right with respect to strut cap base 310. In particular embodiments, travel of about 10 mm is allowed in each direction from a central neutral position. FIG. 7S shows adjustable strut cap assembly 300 in three configurations (from left to right): a neutral centered position, a left shifted position, and a right shifted position.

The positioning and adjustability of an ischial seat pad 340 with respect to a prosthetic socket as a whole is important for overall patient satisfaction and functionality of a socket. Embodiments of the invention include a height or elevational adjustability (FIGS. 7A-7O), this presently described horizontal adjustability, and a wide range of conformational configurations from an inventory derived via shaping steps described below in the context of FIGS. 15A-18. The horizontal adjustability advantageously provides an easily accessible freedom of movement of ischial seat pad 340 with respect to the relatively fixed circumferential position of a strut to which it is attached.

FIG. 7V shows a face view of an ischial seat pad 340T that includes non-Newtonian foam disposed proximally, along the ischium-contacting surface. Non-Newtonian materials 340NNF, such as D3O, have energy absorptive properties that are advantageous for some applications in that while they move slowly in response to application of force below a threshold, they lock up and absorb high impact force, absorbing and dispersing the energy away from the site of impact.

FIG. 7W shows a vertical cross sectional view of an ischial seat pad 340U that includes a temperature controlled or phase change material 340TCM.

FIG. 7X shows a transparent face view of an ischial seat pad 340V that includes an inflatable air bladder 340B. Air bladder 340B can provide an immediate adjustment to the volume and force absorbing features for ischial seat pad 340V.

### Ischial Seat Pads

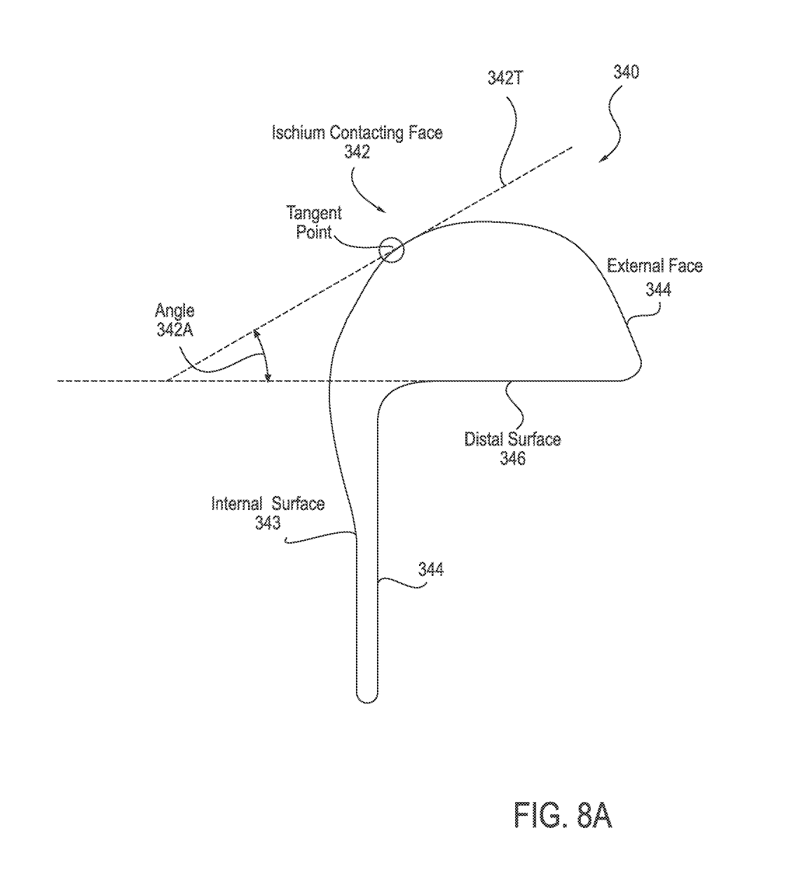

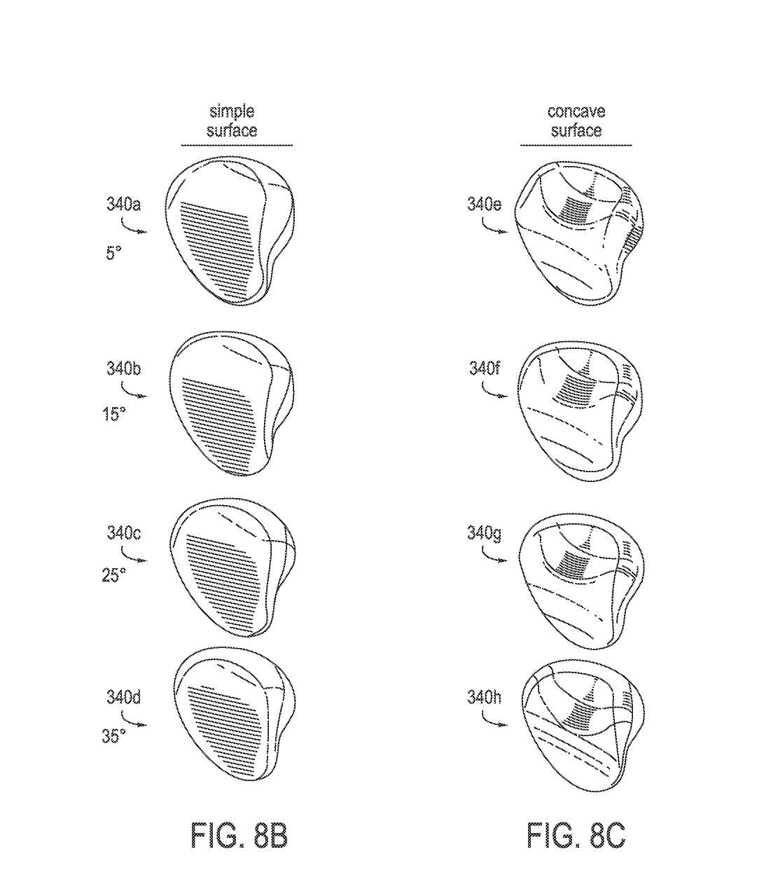

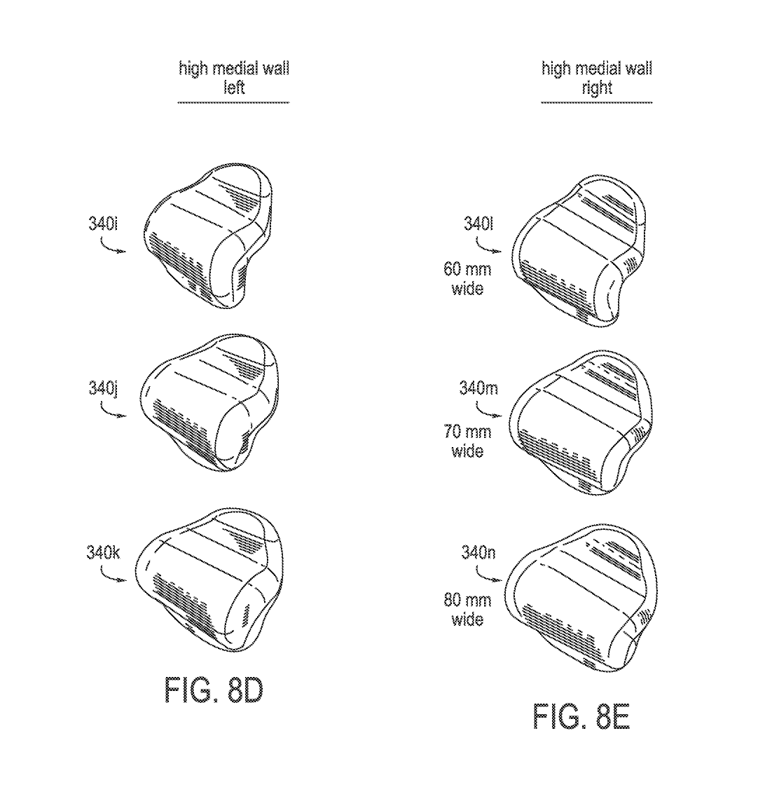

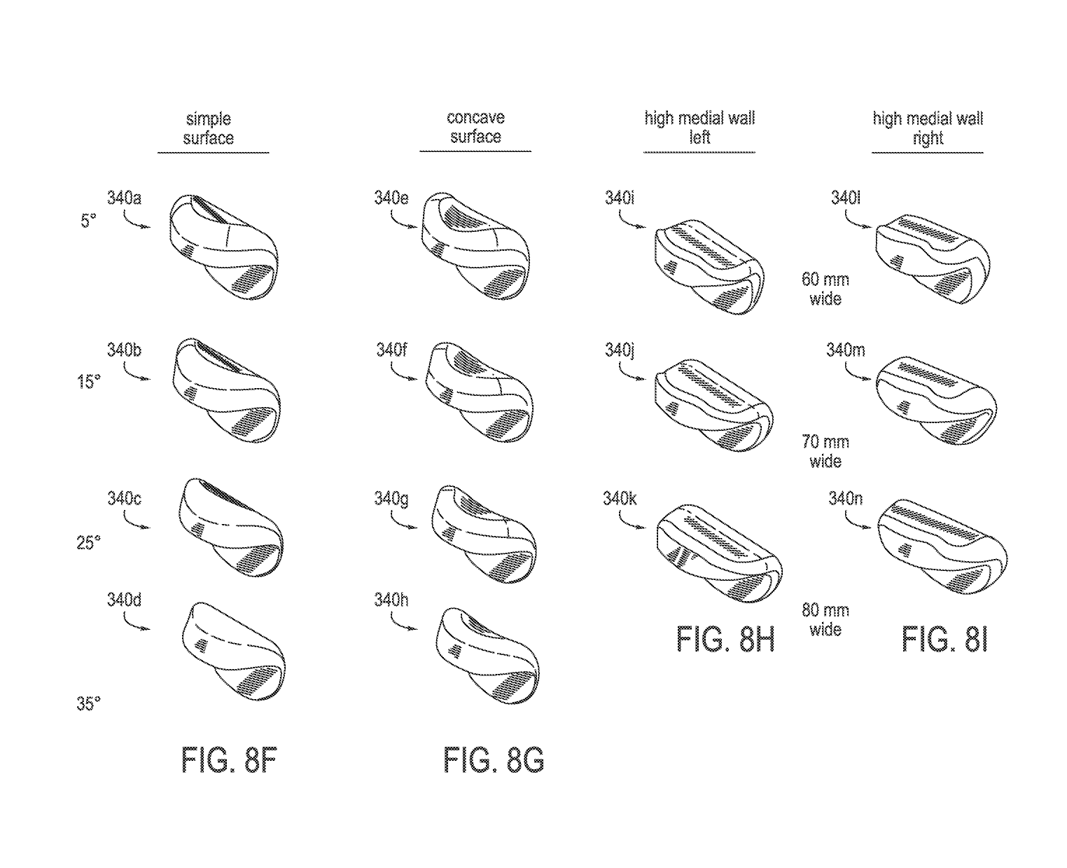

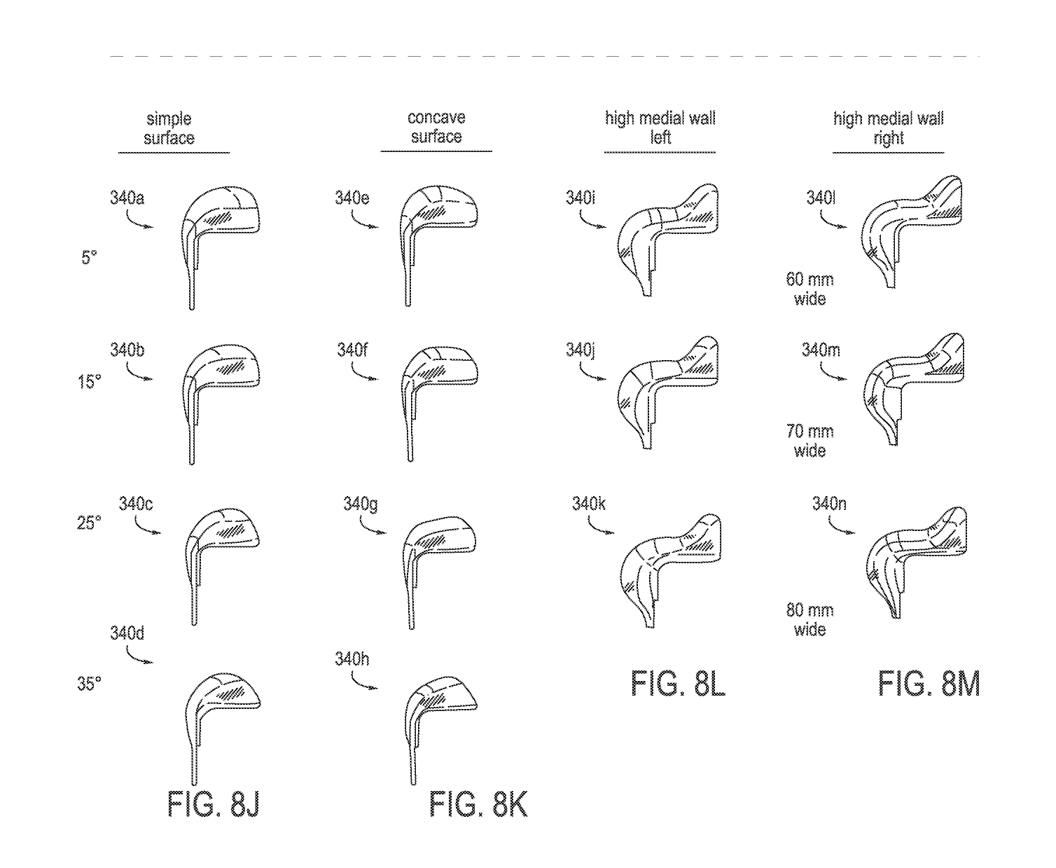

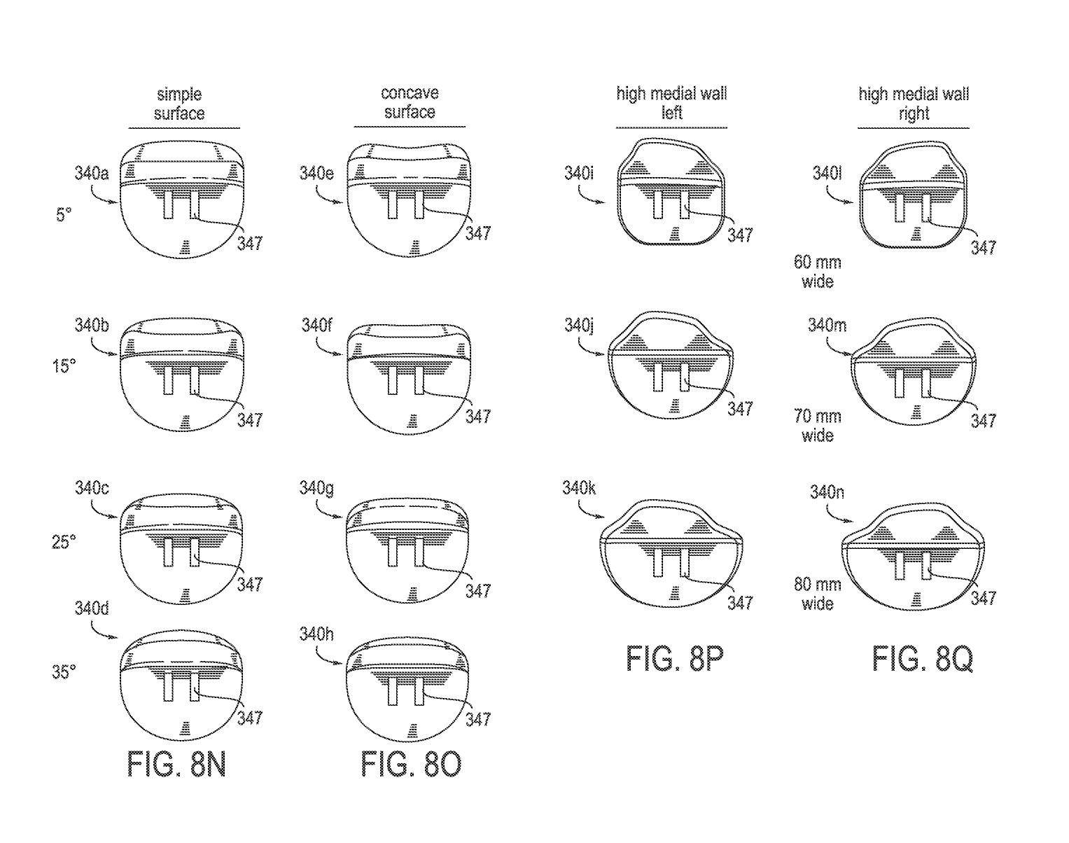

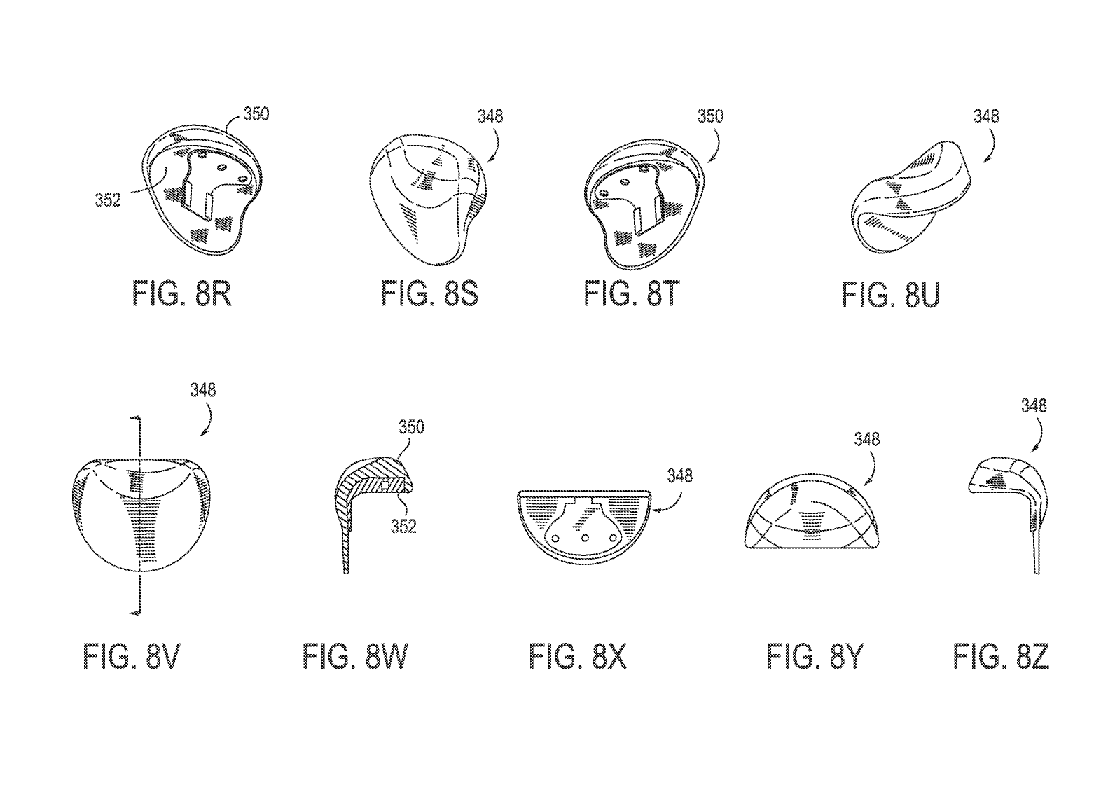

FIGS. 8A-8Q illustrate various alternative embodiments of a seat pad 340, which is available in a large number of modular variations. FIG. 8A is a schematic side view that illustrates the basic features of the seat pad 340. FIGS. 8B-8Q show modular variations of seat pad 340a-340n, in a consistent spatial arrangement, with FIGS. 8B, 8F, 8J and 8N showing a first set of embodiments, FIGS. 8C, 8G, 8K and 8O showing a second set of embodiments, FIGS. 8D, 8H, 8L and 8P showing a third set of embodiments, and FIGS. 8E, 8I, 8M and 8Q showing a fourth set of embodiments. FIGS. 8B-8E are internal perspective (outward-looking) views, FIGS. 8F-8I are external perspective (inward-looking) views, FIGS. 8J-8M are side views, and FIGS. 8N-8Q are internal face (outward-looking) views. Finally, FIGS. 8R-8Z are various views of a seat pad 348 formed from two materials that differ in durometer.

Referring to FIG. 8A, seat pad 340 may include an ischium-contacting surface 342 that, when mounted on a strut, generally faces in an internal and proximal-ward or upper-facing direction. Ischium-contacting surface 342 further includes an external or back face 344, and a distal or bottom surface 346. Label identifier 342 refers generally to the ischium-contacting surface, but varying size and shape embodiments of an ischium-contacting surface are provided. A lower, peninsula-shaped portion (in cross section) has in internal surface 343 (facing internally into a prosthetic socket on which the seat pad is mounted) and an external surface (facing externally from the prosthetic socket) or strut-aligning surface 344.

A first group of embodiments (FIG. 8B) has a simple rounded shape. A second group of embodiments (FIG. 8C) has a concavity disposed along the central axis or the ischium-contacting face, e.g., the concavity aligning in a radial direction with respect to the socket as a whole. A third group of embodiments (FIGS. 8D and 8E) has a high medial or external wall. The seat pads 340a-340h of FIGS. 8B and 8C are laterally symmetrical. The seat pads 340i-340n of FIGS. 8D and 8E, with the high medial wall, are bilaterally asymmetrical. The asymmetry is configured to correspond to corresponding anatomy of the proximal aspect of the patient's ischium. To accommodate the asymmetry, seat pads 340i-340n have left and right versions.

Inasmuch as patient anatomy in the ischial region of the pelvis varies, and inasmuch as an appropriate fit of seat pad 340 against the ischium is important, seat pads 340a-340n are provided in modular variations, differing in size and or shape. The upper rounded aspect of the ischium-contacting face of seat pads 340a-340h has a tangent line 342T associated with the point of steepest angle. Referring to FIG. 8A, the distal or bottom surface 346 of seat pad 340 is substantially flat and horizontal. The angle 342A defined by the vertex of the bottom surface 340 and tangent line 342T can vary in a modular manner, per configurational variations in an inventory. Merely by way of example, an inventory of seat pads seat pads 340a-340h may have models with an angle 342A of 5°, 15°, 25°, or 35°.

The width of a seat pad 340 also can vary in a modular manner. Using seat pas 340i-340n as examples, such seat pads 340i-340n may have models of 60 mm, 70 mm, or 80 mm in width. In spite of these variations in size and shape, positioning features 347 (FIGS. 8N-8Q) on distal surface 346 remain constant. Accordingly, all such modular embodiments of seat pads 340a-340n fit and can be appropriately positioned on a proximal surface 325 of a strut cap base 310.

Referring now to FIGS. 8R-8Z, seat pads may be fabricated from any suitable material by any appropriate method. In some embodiments, for example, seat pads may be fabricated with 3D printing technology. In various embodiments, seat pad 348 may by fabricated using materials that vary in durometer. For example, a base layer 352 may be formed of a high durometer material (about 95D, for example), and an upper layer 350 may be formed from a low durometer material (about 50D-about 60D, for example). 3D printing technology is well suited for fabricating such an article. Aspects of 3D printing technology and its application to fabrication of ischial seat pad embodiments are described in further detail below.

Embodiments of an ischial strut cap assembly may include a seat pad cover 349 that fits over seat pad 340 in a sock-like manner (easy on/easy off). It may be securable by having a closable feature, such an elastic band or drawstring. Seat pad cover embodiments 349 may, in fact be sock-like in terms of a garment, relatively inexpensive, washable, or disposable.

### B. Soft Goods Components

As noted above, improvements for a modular prosthetic socket device and system, as described in U.S. patent application Ser. Nos. 13/675,761 and 14/213,788, include embodiments of soft goods. Soft goods are made of fabrics and soft plastic components and associated connecting and tensioning mechanisms. Soft goods generally serve as elements that connect hardware components together, in a rigging-like manner. Soft goods act as pressure-distributing elements, in conjunction with hardware, distributing pressure away from focal sites of contact between the hardware and the residual limb. Examples of soft goods include, by way of example, strut sleeves, strut caps, brim element covers, jackets, corsets, laces and tensioning lines, and the like. A flexible distal cup, as described herein, may also be considered an example of soft goods. Further, as a consequence of rigging hardware elements together, such as the struts and brim elements, the soft goods integrate these various elements such that they perform as a unified structure.

Soft goods and Brim

Referring now to FIGS. 9A-9F, one embodiment of a modular prosthetic socket 10 is illustrated, showing the soft goods components. In the embodiment shown, the soft goods include a brim 100, tensioning elements (as described further below) and strut sleeves 150. Closely associated with the soft goods is an ischial strut cap assembly 30, also further described below. These various soft goods elements interact with hardware components, rigging them together, so the hardware components function as a functionally unified or integrated structure. As described above, hardware elements provided herein include struts 20, strut connectors 40, and distal base 80. Brim 100 includes two major pieces that, when assembled, wrap around the residual limb. These wrapping pieces include a butterfly segment 110 and a trochanteric segment 130. Various views of soft good items, brim 100 in particular, are shown in FIGS. 7N, 7O, 9A-9F, 10A-10J, and 11A-D. Aspects and embodiments of brim 100 and component segments 110 and 130 are shown in FIGS. 10A-10J in its rolled or circumferentially-arranged configuration. FIGS. 11A-11D depict brim 100 and its components in a laid out plan that associates aspects of the upper boundary of the brim with particular anatomical sites on the residual limb and general pelvic region.

Butterfly segment 110 of brim 100 wraps substantially around the residual limb when being worn by a patient; it is centered at the medial aspect of the limb. A posterior-wrapping wing 122 and an anterior-wrapping wing 121 each extend around to nearly meet the other wing at the lateral aspect of the limb. Lateral or trochanteric segment 130, so-named because it resides over the patient's trochanter occupies a lateral position, and may also be referred to as lateral strut paddle 20L, aspects of which are described further below. These two segments (butterfly wrap 110 and trochanteric segment 130), when assembled together or conjoined by connecting or tensioning elements 140, cooperate to encircle the proximal portion of the residual limb and embrace the surround struts of a modular prosthetic socket 10 embodiment that hosts the residual limb.

Embodiments of medial butterfly segment 110 of a brim 100 include an anterior wing 121, a posterior wing 122, and a spanning section therebetween that includes a posterior-medial valley 124, a medial span 123 and an anterior-medial valley 125 disposed therebetween. In this context, anterior, posterior, proximal, and distal all refer to relative positions of the butterfly segment as it is correctly fitted on a residual limb, and is conjoined with trochanteric segment 130 by way of connecting elements 142, 144. Accordingly, when being worn on a residual limb, medial butterfly segment 110 wraps around the anterior, medial, and posterior aspects of the limb. Trochanteric segment 130 occupies a lateral position on the residual limb. When brim 100 is assembled and being worn, trochanteric segment 130 and medial butterfly segment, collectively, fully encircle the residual limb. The encircling configuration includes area of overlap, i.e., the two lateral edges of trochanteric segment 130 externally overlap the lateral edges of the anterior wing 121 and the posterior wing of butterfly segment 110.

Butterfly wrapping segment 110 further includes an internal aspect or inner surface and 126 an external aspect or external surface 127. In some embodiments, the internal aspect 126 and external aspect 127 are separate fabric pieces prior to being sewn together to form butterfly segment 110. In some of these embodiments, the width of the external aspect is greater than the width of the internal aspect. When sewn together, the assembled butterfly segment 110, by virtue of such disparity in widths, has a conical contour. Conical, in this context, refers to a nominally circular cross sectional profile with respect to a longitudinal axis, with a circumference at the proximal edge that is greater than the circumference at the distal edge. Such a conical profile is advantageous in that it allows brim 100 to bear weight when being worn, and when connecting elements 142, 144 are appropriately tensioned.

Butterfly wrapping segment 110, when correctly fitted on a residual limb, is centered on the medial aspect of the limb; its posterior wing 122 wraps around toward the posterior aspect of the residual limb and its anterior wing wraps around toward the anterior of the limb. Trochanteric segment 130, when correctly fitted on a residual limb, occupies a lateral position on the residual limb. Butterfly wrapping segment 110 has a proximal or upper facing edge 129 that extends across the “top” or “upper” side of the segment, from one side to the other. More particularly, upper facing edge 129 broadly includes the anterior-wrapping wing 121, the posterior wrapping wing 122, and a central or medial section 123. The upper facing edge 129 within the anterior wrapping wing 121 has a concavity or anterior medial valley that abuts the medial section. The upper facing edge 129 within the posterior wrapping wing 122 has a concavity or posterior medial valley 124 that abuts the medial section 123.

These different sections of the upper facing edge 129 of butterfly wrapping segment 110 each have their particular characteristic vertical profile to be appropriate for the anatomy of the patient and for the functioning of the modular prosthetic socket 10 as whole. Each aspect of upper facing edge 129 advantageously allows the an unconstrained use of nearby muscle or tendon and/or provides a point of leverage that balances forces the patient can apply to the socket, thereby supporting control and function of the modular prosthetic socket embodiment 10.

A peak portion 121-P of the anterior wrapping wing 121 rises to approximately the height of an ischial seat and provides a support from which the patient can exert a counter pressure against the ischial seat and allows the patient to remain stabilized on that seat.

The anterior medial valley 125 of the anterior wrapping wing 121 accommodates functioning of nearby adductor muscles. On its medial side, the relatively sharp drop in the contour accommodates the ramus tendon.

The medial portion of butterfly wrapping segment 110 hosts an ischial or medial strut channel 114, and also is the site of a locking mechanism for a telescopic adjustable-height strut cap assembly that is described in further detail below. An anterior strut enclosure pocket 116 and posterior strut enclosure pocket 118 are also disposed within butterfly wrapping segment 110.

The posterior medial valley of the posterior wrapping wing 122 accommodates functioning of the nearby gluteal muscles. The peak portion 122-P of the posterior wrapping wing 122 provides posterior lateral support for the patient.

Trochanteric segment of lateral strut cap 130 provides lateral support and a high point from which to suspend A-shaped ladder lock assembly that allows tensioning to apply an upward pull on the butterfly wrapping segment 110 brim 100.

Tensioning or connecting elements include a V-shaped loop-lock assembly 112 disposed on external surface 127 of butterfly wrapping segment 110, A-shaped ladder lock assembly on the external surface 127 of trochanteric segment 130, and a two-part tensioning belt and connecting ratchet buckle 144. Belt 142 and connecting ratchet buckle 144 connect butterfly segment 110 and trochanteric segment 130 together, and allow for variable tensioning to be applied.

Customization of the fit of a modular prosthetic socket 10 to an individual patient occurs through one or more approaches: (1) selection of appropriately sized and shaped fixed form components and (2) selection of appropriately sized and shaped thermally reformable components. Brim embodiments 100 may be simply sized as small, medium, and large, without changing the basic contours of the upper border of the brim. Some embodiments of a brim 100, being formed from low density polyethylene (LDPE), may also be thermally reformable. Reforming may be performed against an in animate positive mold, by manipulation in the hands of a prosthetist or possibly the user, as well as by way of a direct molding approach, as described elsewhere in the context of embodiments of a flexible distal cup. A third type of fitting is entirely in the hands of the patient, by way of manually adjusting the tensioning elements to personal preference easily, and as frequently as desired.

Referring to FIG. 9F, some embodiments of soft goods technology further provide strut sleeves 150, which can be arranged over each a portion of each of the struts 20 of modular prosthetic socket 10. As described elsewhere, an embodiment of a modular prosthetic socket 10 that includes four struts, may include ischial strut 20M, anterior lateral strut 20AL, anterior medial strut 20AM, and posterior strut 20P. Each of the struts can be covered by a strut sleeve 150, each strut sleeve being configured to fit the length and width of the enclosed strut, each sleeve having a proximal opening and a distal opening.

By way of introducing soft goods and their role in distributing pressure, in some embodiments of a modular prosthetic assembly, a pressure-distributing element may include or be formed form any a polymer or a soft good material. By way of example, a polymer may include any of ethylenevinylacetate (EVA), low density polyethylene (LDPE), or a blend or a copolymer thereof, or any suitable polymer. Pressure-distributing elements that include a polymer typically have sufficient strength and resilience that the element bears pressure well without compromising the form of the element. Such embodiments may be able to distribute pressure impinging from struts with substantial uniformity across the surface of the element. In embodiments that include a soft good material, such soft goods may, by way of non-limiting example, include any of a fabric or a leather material. Such fabric may include any of a knit fabric, a woven fabric, a non-woven fabric, or any suitable fabric that includes either natural or synthetic materials, and may include additives or impregnated materials that add desirable properties to the fabric.

Various embodiments of pressure-distributing arrangements that include soft goods in association with hard structural socket components are provided U.S. patent application Ser. No. 14/213,788. That application is incorporated herein by this reference, but aspects of it will now be referred to specifically, as for example, paragraphs 328-330 and FIGS. 22K and 23A-23C. FIG. 22K is a fabric sleeve fitted over a single strut with bilateral attachments suitable for attaching either to a tensioning member or an adjacent strut sleeve. A fabric sleeve may be placed over a strut and any associated pressure-distributing element. Sleeves may be advantageous for their “soft-good” character that provides an interface friendly to the residual limb, and which further may include attachment features that can support tensioning elements and/or fixed-length connections to other struts or other pressure-distributing elements.

FIGS. 23A-23C of U.S. patent application Ser. No. 14/213,788 show views of modular prosthetic socket embodiments, each with a different arrangement of pressure distributing elements, including strut caps and strut brims. FIGS. 23A-23C each show a prosthetic socket having struts, distal base, and an embodiment of a distal cup. They differ with regard to their respective arrangements of tensioning elements and pressure-distribution elements. FIG. 23A is prosthetic socket fitted with strut caps as a pressure distributing element, and a tensioning band and an adjustment mechanism arranged circumferentially around the struts. FIG. 23B is prosthetic socket fitted an integrated brim as a pressure distributing element, and a tensioning band and an adjustment mechanism arranged circumferentially around the struts.

FIG. 23C of U.S. patent application Ser. No. 14/213,788 is prosthetic socket fitted with laceable corset as a combined pressure distributing element and tensioning mechanism that is arranged circumferentially around the struts or more generally within or proximate the circumference nominally defined by the struts. Tension adjustment mechanism is shown at the proximal end of the lacing mechanism. In related embodiments, there may be more than one tensioning mechanism, allowing tensioning to independently adjustable in different longitudinal sections of the corset. This arrangement, as noted above, has a soft-goods character that is friendly to the residual limb. A corset such as this is typically fabricated from fabric or leather, and may also be considered as a type of sleeve, or include specific aspects that act as sleeves, enclosing or wrapping the strut, or wrapping one of more pressure-distributing elements associated with a strut, such as a strut cap or brim element.

Various features of a modular prosthetic socket 10 distribute pressure and absorb body weight, these structural features primarily include embodiments of the distal base 80, struts 20, and brim 100 elements, embodiments of medial brim 110 and trochanteric lateral pad 130, and ischial strut cap assembly 30 can bear weight. Flexible distal cup 200 may also bear some weight, as substantially shared or transmitted to a distal base 70.

A tensioning arrangement or system is associated with brim 100 that includes A-shaped Ladder Lock assembly 132 on trochanteric segment of brim, V-shaped Loop-Lock assembly 112 of butterfly wrap segment 110, as well as tension belt 142 and ratchet buckle 144. These elements of the tensioning system cooperate to tighten and loosen the brim around the residual limb when modular socket 10 is being worn by a patient. Two levels of adjustment are provided: a macro adjustment is effected by manipulating the A-shaped ladder lock assembly, and a micro adjustment is effected by manipulating ratchet buckle 144 as it tightens or loosens the two converging ends of tension belt 142.

Brim 100 includes an ischial (medial) strut enclosure channel 134, as well as three strut enclosure pockets 136 (136AL, 136PM, and 136P, in accordance with the four struts). These enclosure elements effectively allow transmission of tension applied to brim 100 to the set of struts. (The arrangement also effectively allows transmission of force absorbed by the struts to the brim.) Accordingly, adjustments made by manipulation of these tensioning elements (as listed above) is applied to the confines of the space immediately within the brim, but extend further distally as well, as the tensioning applied to the brim is transmitted to the struts.

Brim 100, as a whole, when assembled, has a generally conical shape (wider at the proximal end, narrower at the distal end). This shape conforms to the general shape of a residual limb. In addition to serving the function of fitting the residual limb, a consequence of the application of tension to the conically shaped brim is to urge the residual limb upward. As the brim urges the residual limb upward, the body weight of the patient resists. Accordingly, tensioning the tensioning system causes the brim to incrementally bear more weight. Further still, if considering the more distal structural elements that bear weight (the struts and the distal base), the effect of tensioning the tensioning system is to cause a general shift of weight bearing upward (proximally) from the distal base 80 toward the brim.

This aspect of the effect of increasing or decreasing tension on the brim, by way of adjusting the tensioning system forms the basis for a method by which a patient can adjust the distribution of weight upward, toward the brim 100, or downward, to distal base 80.

### C1. Composite Polymer EVA/PCL in a Monolithic Form and a Flexible Distal Cup Made Therefrom

Embodiments of the technology further include a flexible distal cup for a prosthetic socket, as exemplified by embodiments of modular prosthetic sockets and their components as provided herein. An inner liner may also be referred to as a distal cup. “Distal” refers to the position of the article within an assembled socket; “cup” refers to the general shape of the article and the manner in which it supports the distal end of a residual limb. Attributes of flexible distal cups include a flexural rigidity that is sufficient to fulfill various desired mechanical capabilities at the interface between a patient's residual limb and hard structural elements of a prosthetic socket, such as struts and a distal base. The flexible distal cup needs to be able to effectively distribute pressure away from pressure foci, as represented by interface sites between such hard structural elements and the patient's limb. On the other hand, the flexural rigidity of flexible distal cup embodiments needs to be sufficiently low that it comports well against the residual limb, does not impede appropriate tissue movement, and is subjectively comfortable for the patient.

Further, embodiments of flexible distal cups are compositionally constituted such that they can be thermally reformed against the patient's residual limb, to establish the form of the flexible distal cup as one that custom-fits the residual limb. Thermoplastic compositions are thus advantageous in that they can be thermally reformed. However, since the thermal reforming is to be done directly against the patient's body, the thermoplastic softening temperature of the flexible distal cup composition needs to be tolerable to the patient, such as a range of between about 125° F. and about 160° F. In terms of softening temperatures of the broad class of thermoplastics, this is a relatively low temperature.

Embodiments of the technology include a composite polymer composition and an article formed from or including such composition. The composition embodiment includes (1) ethylene-vinyl acetate (EVA) copolymer and (2) polycaprolactone (PCL). (The composition as a whole can be referred to as EVA/PCL.) EVA accounts for between about 45% and about 55% of the composition by weight. The vinyl acetate portion of the EVA polymer accounts less than about 25% of the EVA by weight. The polycaprolactone accounts for between about 45% and about 55% of the composition by weight. Some embodiments of the composition, as described, are, that is, the component polymer populations are well mixed, effectively forming a homogeneous composition. Embodiments of the EVA/PCL composition have a reformable or softening temperature of between about 125° F. and about 160° F., and a flexural modulus of about 3,500.

In particular embodiments of the composition, the molecular weight of the polycaprolactone (PCL) within the composition ranges between about 37,000 and about 80,000. In particular embodiments of the composition, the vinyl acetate proportion of the EVA comprises about 12% to about 28% of the EVA by weight. Accordingly, the weight ratio of PCL to EVA is in the range of about 0.43 to about 2.33. The weight/weight ratio of 0.43 represents a relative presence of about 43% PCL and about 57% EVA; the ratio of 2.33 represents a relative presence of about 70% PCL about 30% EVA.

Other agents or moieties may be included within the composite polymer composition without significantly altering material properties of the composition. Such agents or moieties may include, by way of example, color agents or anti-stick agents.

Many physical articles may be formed from the composition as summarized above. Some of these articles may be used in medical devices or components thereof that interface with the body, such as prosthetics, orthotics, casts, splints or braces. These devices take advantage of the material properties that include a body-friendly flexibility, but also having sufficient integrity to support a body portion when the material is formed into a device. Such medical devices typically have contoured surfaces or hollow regions that are complementary to a portion of the body. One particular embodiment of the technology is a flexible distal cup device for a prosthetic socket that manifests as an elongate, cup-shaped member configured to fit around a residual limb of a patient, and to be disposed within a host prosthetic socket. To recite some of the features of the composition summarized above, the composition of flexible distal cup embodiments includes or consists of (1) ethylene-vinyl acetate (EVA) copolymer and (2) polycaprolactone (PCL). The EVA accounts for between about 45% and about 55% of the composition by weight. The vinyl acetate portion of the EVA polymer accounts less than about 25% of the EVA by weight. The polycaprolactone accounts for between about 45% and about 55% of the composition by weight. Some embodiments of the EVA/PCL composition have a flexural modulus of about 3,500.

The EVA/PCL composition has a reformable temperature of between about 125° F. and about 160° F. The reformable temperature may also be referred to as a softening temperature or a manually reformable temperature. This temperature, regardless of the terminology, refers to the temperature at which an article comprising the composition becomes soft enough that it can be reshaped, maintain its structural integrity, and when cooled, maintains the reshaped form.

Embodiments of a flexible distal cup typically are garment-like in that they fit or conform to a portion of a residual limb, having a substantially closed distal end, an open proximal end sized and configured to receive a patient's residual limb, an internal surface and an external surface. The height (distal end to proximal end) of flexible distal cup embodiments can vary with respect to the length or height of a prosthetic socket that hosts the flexible distal cup. In some embodiments, the height of the flexible distal cup, when positioned within the prosthetic socket is equal or nearly equal to the prosthetic socket. In other embodiments, the height of the flexible distal cup is substantially less than the height of the prosthetic socket.

In some embodiments, the distal end includes a hardware insert embedded within or bonded to the flexible distal cup. This relationship may be established in an over molding process, wherein the flexible distal cup is formed in a mold that is placed over the hardware insert. Embodiments of the hardware insert may include connective features, configured to mate with distal hardware components. Further, embodiments of the hardware insert may include one or more valve units, configured to controllably allow passage of ambient air and moisture included therein. Such valves may include 1-way valves or 2-way valves. These connective features and valves may be positioned so their functionality is directed laterally or distally from the flexible distal cup.

In the context of modular prosthetic system, modular components can vary in shape and/or dimensionality. As described in U.S. patent application Ser. No. 14/213,788, such modular components may include distal base plates, strut connectors, struts, and brims, among others. Despite variation in shape and dimension, modular components retain common connecting features that allow a prosthetic socket to be assembled. As with these other modular prosthetic socket components, so also may flexible distal cups be provided in an inventory or group of components that vary in size and/or shape.

U.S. patent application Ser. No. 14/310,147 of Hurley and Williams, as filed on Jun. 20, 2014, provided several embodiments of a moisture management roll on liner. Various features described therein could be advantageously applied to the presently described flexible distal cup, consequently imparting moisture management capabilities thereto.

Some embodiments of the technology are directed to a method of making a flexible support device for a residual limb. Such method includes compounding pellets of ethylene-vinyl acetate (EVA) copolymer and pellets of polycaprolactone (PCL), wherein EVA comprises between about 45% and 55% of the composition by weight, and wherein vinyl acetate comprises less than 25% of the EVA by weight, and wherein polycaprolactone comprises about between about 45% and 55% of the composition by weight. Together, of course, the combined weight of EVA and PCL is substantially equal to 100%. Embodiments of the method further include molding the compounded material into a device of desired shape and size. By way of example, one particular flexible support device can take the form of a substantially tubular article having a closed proximal end, an open distal end, a length, and a circumference at the distal end, all dimensions being appropriate for receiving the distal end of a residual limb.

Embodiments of the technology are also directed to a method of thermally reforming a flexible support device for a residual limb, the device including or consisting of an EVA/PCL composite polymer material. Such method includes heating the formed article to a sufficient temperature and for a sufficient duration that the composite polymeric material becomes pliable. The method continues with applying sufficient and appropriately directed force to the pliable composite polymeric material such that the initial shape of the formed article changes toward a desired reformed shape.

In particular embodiments, a flexible support device, such as a flexible distal cup for a prosthetic socket, may be fabricated in a variety of sizes and shapes, to form an inventory of flexible distal cups from which any of a large range of residual limbs can be fitted.

In thermally reforming a flexible distal cup, as above, in order to custom fit a residual limb, one approach is to select a flexible distal cup from an inventory that is undersized, for example having a nominal diameter that is about 5% less than the nominal diameter of the residual limb. Embodiments of the EVA/PCL composite polymer material, as described above, when taken to a characteristic thermal softening temperature are pliable and elastic, and expandable to accommodate the nominally larger residual limb dimensions. In general, a conformable fit is advantageously approached by way of such stretching of the EVA/PCL composite polymer material, rather than compressing a thermally softened material to conform to a smaller dimensioned residual limb. Thermal reformability is a property of thermoplastics that creates a new form without damaging the integrity of the material. Thermal reforming can be done for an indefinite number of times, if done under appropriate conditions.

“Direct molding” is a term in prosthetics and related medical practices that refers to a method of molding an article directly onto a body part. The “directness” of the molding contrasts with molding an article over an inanimate positive mold standing in for a body part. The use of an intervening thermal insulation to protect the body part from the heat of a thermally softened article is common, and does not contradict the use of “direct” molding in the sense of this term of art. Accordingly, a method of direct molding of a flexible distal cup over a distal portion of a residual limb is provided herein. In some embodiments of the method, a flexible distal cup is formed from an EVA/PCL composite polymer, as described herein, but the scope of the method includes the direct molding of a flexible distal cup fabricated from any suitable material that is amenable to such direct molding over a body part. Direct molding takes advantage of the thermal reformable properties of thermoplastics; if done under appropriate conditions, direct molding can be done for an indefinite number of times, as may be needed to maintain the fit of a flexible inner over a residual limb that can change in shape or dimension.

Samples for testing ethylene-vinyl acetate (EVA) and polycaprolactone (PCL) composite polymer material were prepared by compounding procedures. Compounding is the process of mixing the two precursor plastics to form a well-mixed, homogenous whole. This is done mechanically, by various approaches and under various conditions, and typically ends with the compounder re-pelletizing the plastic. One of the main challenges of compounding is that identifying process parameters that are sufficient to effectively mix the plastics, but not subject them to a level of stress to the extent that polymer chains are disrupted. Such disruption represents a degradation of the compound, and a loss of the physical attributes of the compound that are being sought.

A small-scale batch compounder was built to perform high shear mixing of the EVA/PCL blends. The compounder included two stainless steel barrels connected by a threaded junction: a proximal feed barrel and a distal mixing barrel. The proximal feed barrel (14 inches in length, ¾ inch diameter) contained a helical feed screw, the shaft of which exited the barrel through an end fitting with a hole sized for the shaft. The feed screw was manually rotated by means of a handle. A hole was drilled in the top surface of the barrel over the proximal end of the feed screw. A funnel was placed into the hole to allow for the feeding of the polymer pellets into the feed barrel. Turning the handle and rotating the feed screw advanced the pellets distally into the mixing barrel.

The distal mixing barrel (13 inches in length, ¾ inch diameter) contained a 12-element stainless steel static mixer. The static mixer design included alternating angled elements or baffles that continuously blend the materials. A static mixer can produce patterns of flow division and radial mixing. Two temperature controllers with two temperature zones apiece were employed to control the temperature in the both the feed and mixing barrels. Four high-temperature heating tapes were used to heat each barrel, with the controller set-up allowing for four heating zones, two in the feed barrel and two in the mixing barrel.

During the course of developing the proper conditions for compounding sample ethylene-vinyl acetate (EVA) and polycaprolactone (PCL) composite materials, various temperature regimes for the four temperature zones were tested. The resultant polymer blend was examined as it exited the mixing barrel for signs of burning (darkening or browning of the mix) or signs of inadequate mixing (distinct non-homogeneous regions, variable melt temperature). The goal was to maximize the processing temperature to provide the lowest viscosity for mixing without degrading the material. Temperatures for the four zones in the range of 130°-160° C. were found to yield good blending without adverse affects on the polymer blend. Temperatures at or near the lower end of the range were used for the two heating zones in the feed barrel and temperature at or near the higher end of the range were used for the two heating zones in the mixing barrel. “J”-type thermocouples were attached to the outside of the barrels to provide temperature feedback to the controllers.

Approaches to large scale compounding are anticipated to be different than those for the small-scale approach outlined in this example. Conventional compounding utilizes rotating single or double screw machines wherein the screws are specially designed for mixing and many different screw designs are available to the compounder depending upon the materials being mixed and their properties. A rotational system provides significantly more shear than the laboratory system used and should therefore result in very highly homogeneous mixtures of the polymers. The temperatures required for conventional compounding will also depend on the specific compounding machine and level of shear that may be applied.

Two sample EVA/PCL composite polymeric compositions, compounded by methods described above, were prepared.

Sample A Starting Materials:
55 wt % polycaprolactone from Perstorp Holding AB (Sweden); CAPA 6800 product (molecular weight of 80 KD).
45 wt % ethylene vinyl acetate copolymer from DuPont Corp; Elvax 460 product (18% vinyl acetate by weight).

Sample B Starting Materials
55 wt % polycaprolactone from Perstorp Holding AB (Sweden): CAPA 6800 product, (molecular weight of 80 KD).
45 wt % ethylene vinyl acetate copolymer from DuPont Corp: Elvax 660 product (12% vinyl acetate by weight.

The polymers in each sample were blended to homogeneity such that they showed uniform material properties, and they were then tested for physical parameters. Melt temperatures were determined by an automated optical melting point apparatus.

Characteristic Melt Temperature

| | Onset Temp (° C.) | Single Point Temp (° C.) |
|---|---|---|
| Sample A | 60 | 72 |
| Sample B | 66 | 79 |

Flexural modulus was determined using ASTM D790-07, Standard Test Methods for Flexural Properties of Unreinforced and Reinforced Plastics and Electrical Insulating Materials.

Characteristic Physical Properties

| | Flexural Modulus | Flexural Modulus |
|---|---|---|
| | at room temp (PSI) | at 34° C. (PSI) |
| Sample A | 22,916 | 18,350 |
| Sample B | 21,715 | 17,507 |

C2. Flexible Distal Cup: Connection to a Distal Base, and being Positioned within a Prosthetic Socket

Embodiments of the technology include a flexible distal cup 200 for a prosthetic socket, as exemplified by embodiments of a modular prosthetic socket 10 and its components as provided herein. The role of a flexible distal cup is to fit around the distal end of a residual limb, to support it, to interface between the residual limb and structural features of the prosthetic socket (e.g., struts, distal base), to distribute pressure away from points of contact between the residual and those structural features, to be able to bear at least some weight, and to be thermally reformable.

Flexible distal cup 200 does not cleanly fall into one and only one category of hardware or soft goods, as described elsewhere as a useful organizing principle for types of modular components in a modular prosthetic socket system. Flexible distal cup 200, for example, has a structural role in the socket and an ability to bear weight that is uncharacteristic of other types of soft goods. At the same time, it has a pliability and flexibility that is uncharacteristic of other structural elements of the frame.

It is advantageous for embodiments of a flexible distal cup 200 to be thermally reformable at a temperature range that a patient can tolerate when the liner is placed over the residual limb. The function of thermal reforming is to custom fit a flexible distal cup 200 to the residual limb. Custom fitting optimizes the interfacing and pressure distribution role of the flexible distal cup. Aspects of the composition of flexible inner 200 embodiments, and physical characteristics of the composition are described above.

### Flexible Distal Cup

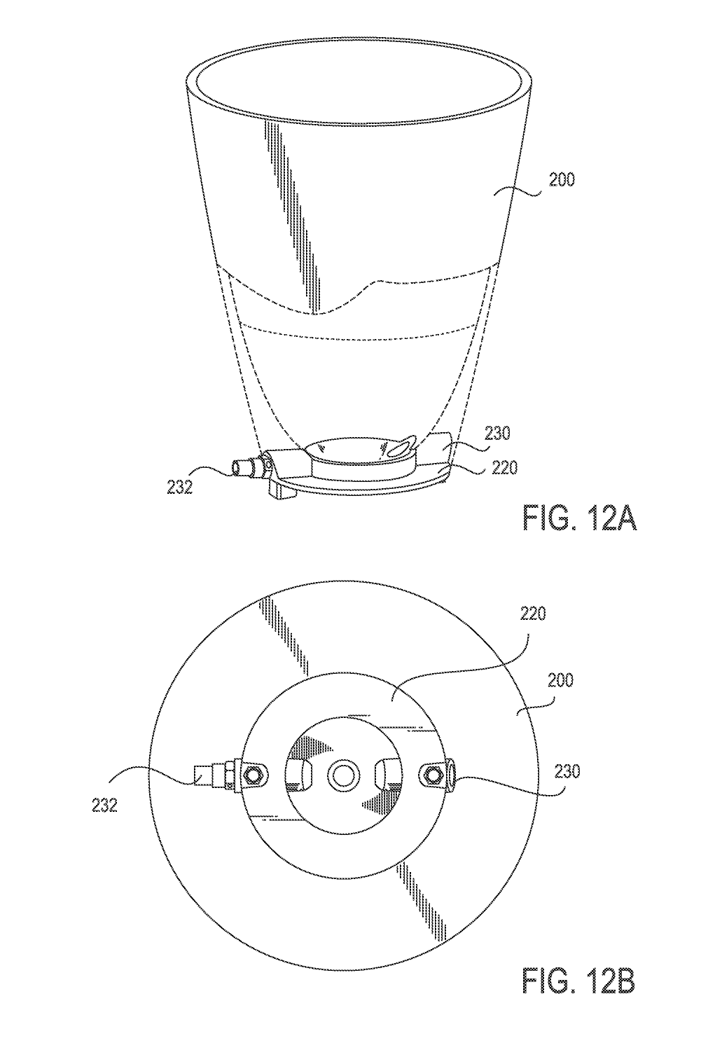

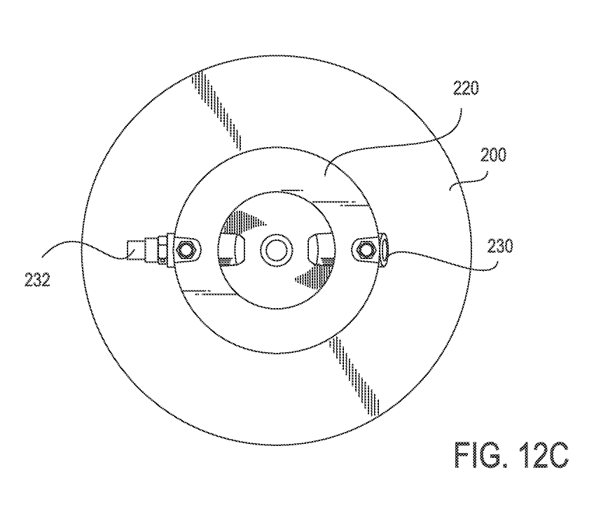

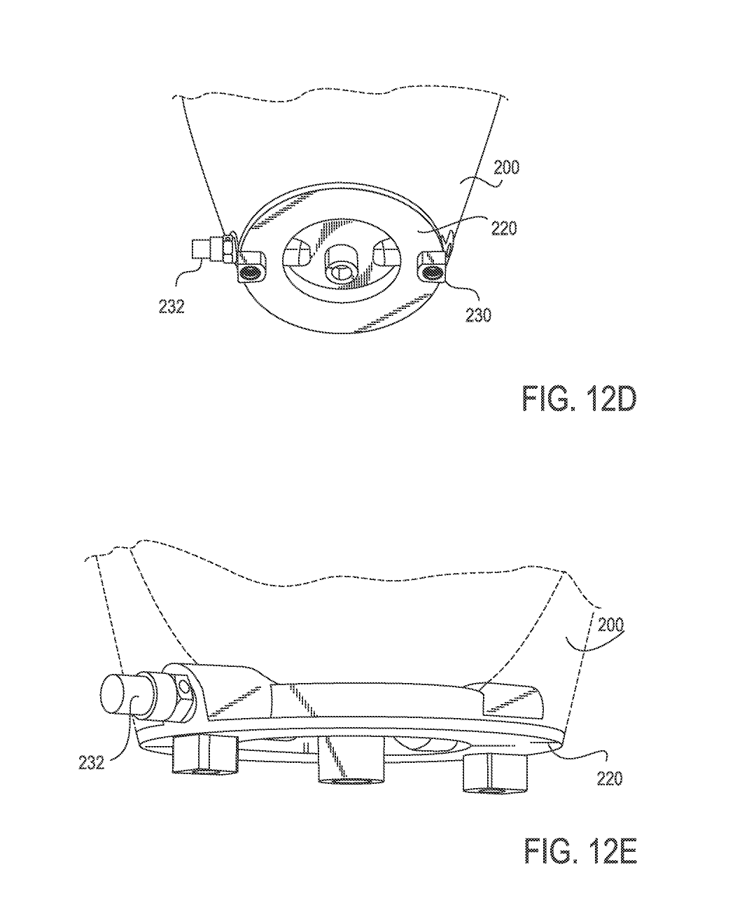

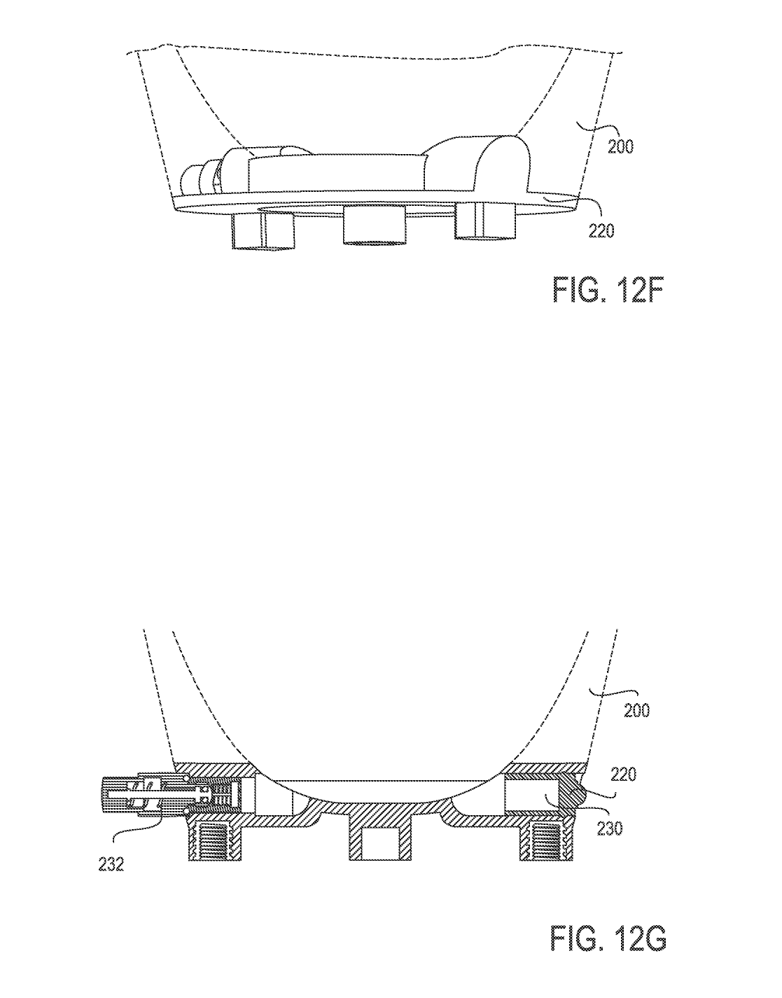

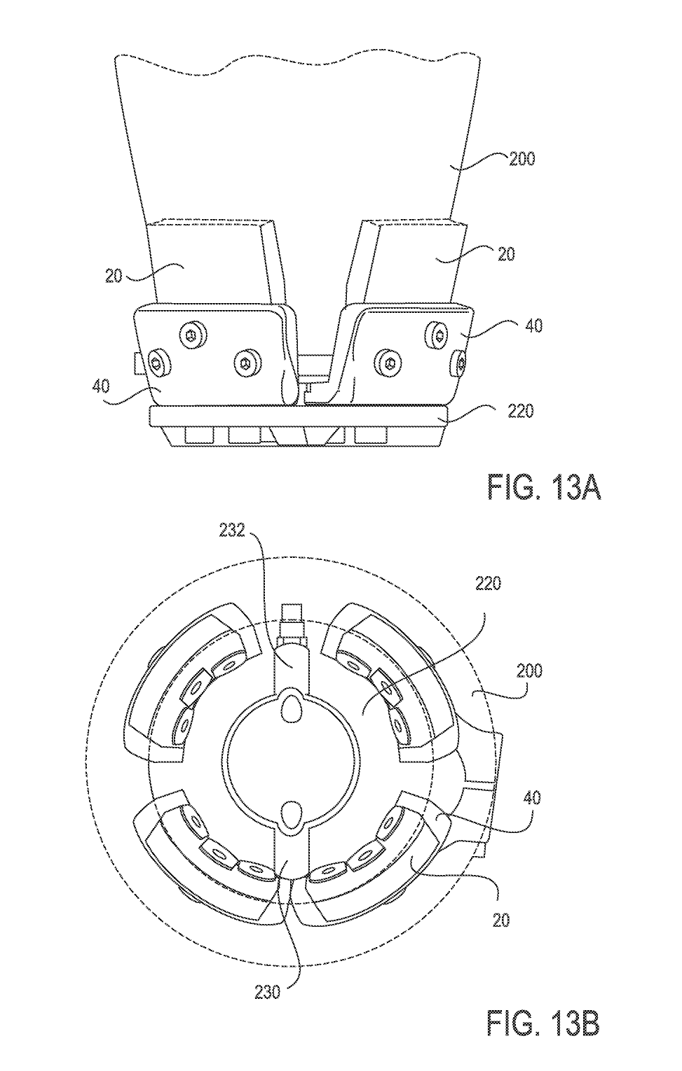

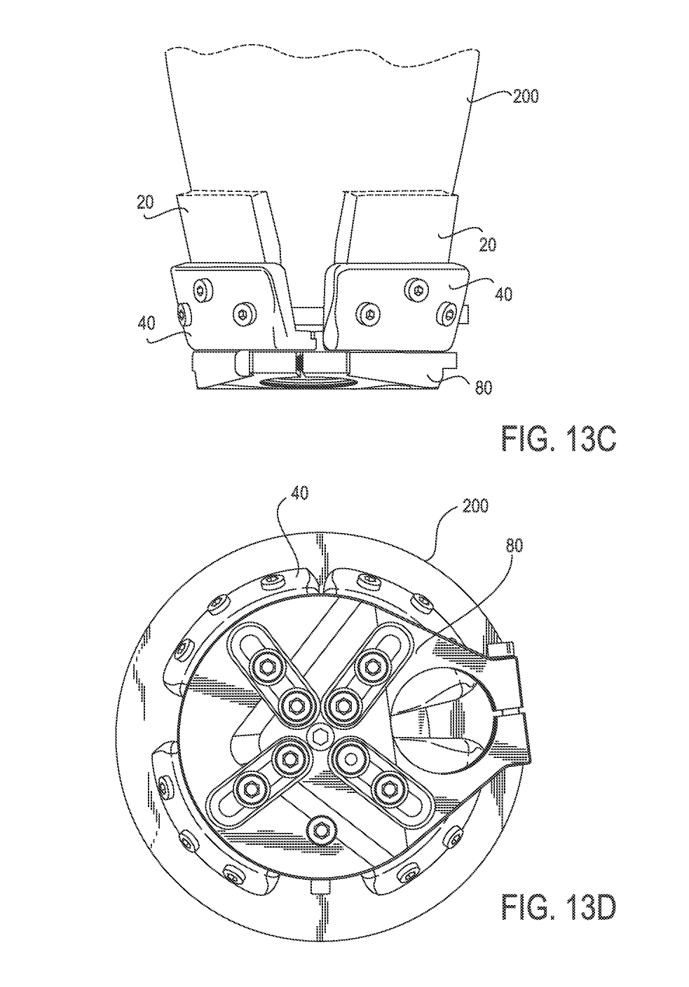

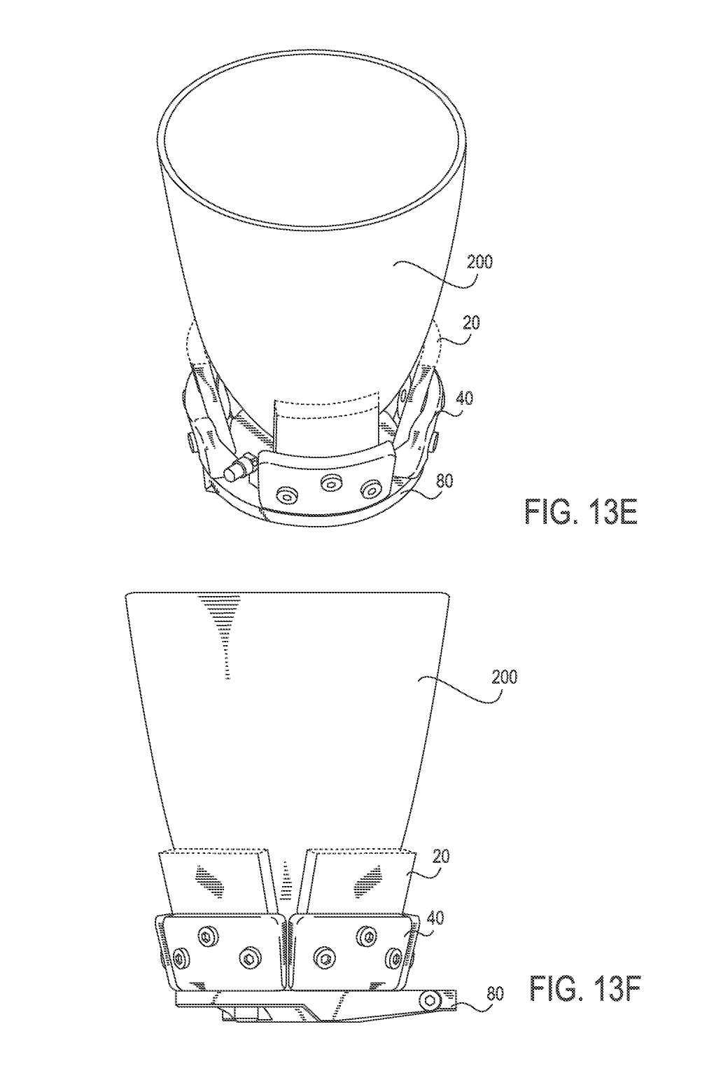

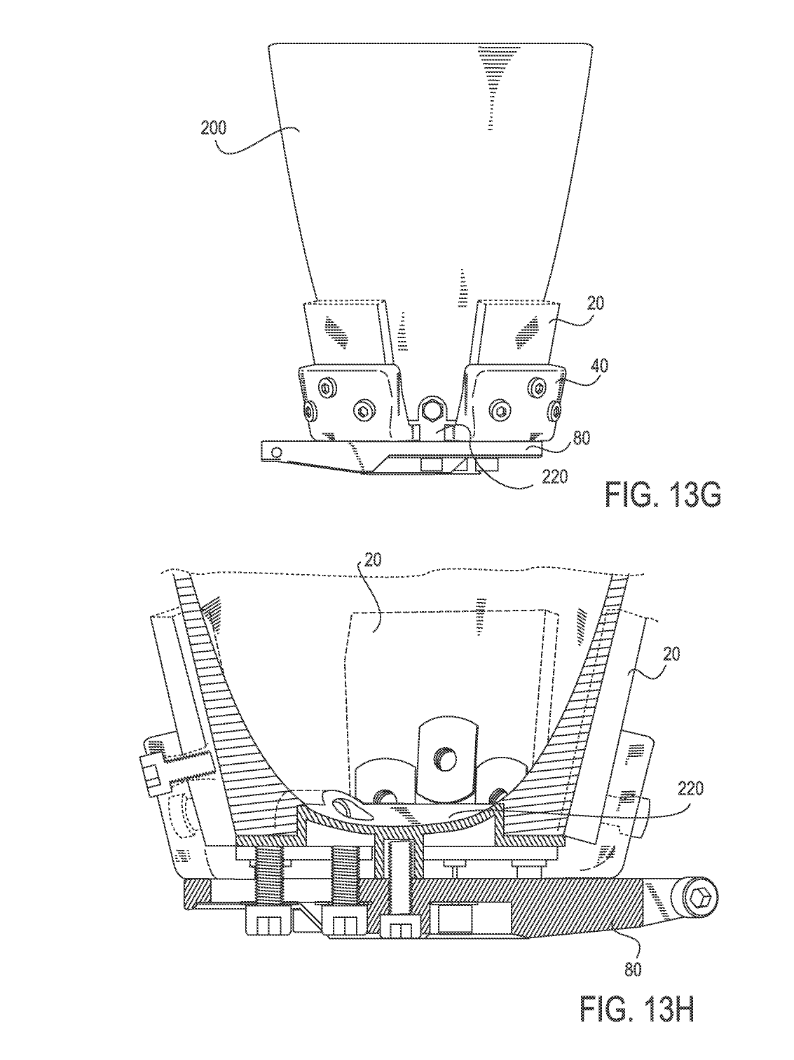

This section now turns to structural aspects of a flexible distal cup 200 and to an anchoring insert piece that can be embedded therein or bonded thereto, at a distal end 208 of the flexible distal cup. Flexible distal cups typically conform to the distal end of a residual limb, and are configured to be disposed into the distal end of a prosthetic socket. Some embodiments of a flexible distal cup are particularly adapted to being connected to structural components of a prosthetic socket. Aspects and embodiments of flexible distal cup 100 are shown in FIGS. 12A-13H.

Flexible distal cup embodiments are generally cup-shaped, with an open distal end and a closed proximal end. They typically have a conical or distally tapering profile, but may be fabricated into a variety of shapes and sizes, as, for example, could be included in an inventory. Label identifier 200 will refer to any flexible distal cup, regardless of particulars of size and shape. Some embodiments have a relatively short form 200B, and enclose only the most distal portion of a residual limb. Other embodiments have a relatively long form 200A, and enclose a substantial portion of the length of a residual limb, as the limb is disposed within a prosthetic socket. Flexible distal cup embodiments 200 include circumferentially complete wall with an internal surface 202 and an external surface 204, an open proximal end 206, and a distal end 208 at which an anchoring insert 202 is disposed. The flexible distal cup wall varies in thickness, being relatively thin at the proximal end and substantially thicker at the distal end.

Anchoring insert 220 is disc-shaped, having a proximal surface 221 and a distal surface 222. Proximal surface 221 has a central or inner circular region 221-I and an outer circumferential region 221-O. As disposed at the distal end 208 of flexible distal cup 200, central region 221-I is exposed to the cavity internal within the confines of flexible distal cup 220, while outer region 221-O is bonded against the distal end of flexible distal cup 200. In particular embodiments, anchoring insert 220 is fabricated from acrylonitrile butadiene styrene (ABS).

Two valves are incorporated into the illustrated embodiment of anchoring insert 220 to handle movement of air in and out of the confines of flexible distal cup 200 when being worn (or when being donned or removed) by a patient. A 1-way expulsion valve 230 is disposed on the lateral side 223L of anchoring insert 220, and a 2-way button-controlled valve 232 (button 233) is disposed on the medial side 223M of anchoring insert 220. In alternative embodiments, theses valve functionalities could be incorporated into a single valve, and located in other positions.

The 1-way valve 230 allows expulsion of air from the confines of the flexible distal cup when the patient is donning the flexible distal cup, and also allows expulsion of air as it can accumulate during normal wear. The 2-way valve opens at the push of a button, and allows movement of air in both directions. Allowing the entry of air into the confines of flexible distal cup 200 is helpful for the patient when removing the flexible distal cup, which otherwise can be resistant to removal because of the vacuum created by the sealing effect of the flexible distal cup against the residual limb. Valve 230 has an entry disposed within the inner region 221-I of the anchoring insert 220, exposed to the environment within the flexible distal cup 200, and a exit opening on lateral edge 223L, which is exposed to the external environment. Valve 232 has an entry/exit opening disposed within the inner region 221-I of the anchoring insert 220, exposed to the environment within the flexible distal cup 200, and an entry/exit opening on medial edge 223M, which is exposed to the external environment.

Anchoring insert 220 further includes a central mounting pedestal 240C projecting distally from distal surface 222 that includes a bolt through hole, as well as at least two peripheral mounting pedestals 240P with bolt through holes. These mounting pedestals align with central bolt hole 86 and peripheral bolt holes 87 of distal base 80. When assembled, bolts enter bolt holes 86 and 87 from the distal side 82 of distal base 80 and then thread into bolt holes within mounting pedestals 240C and 240P of anchoring insert 220 (of flexible distal cup 200). When tightened, these bolts secure flexible distal cup 200 against distal base 80.

D. Translating Residual Limb Data (Size, Shape, and Tissue Density) into Custom Shaped Struts for Assembly into a Modular Prosthetic Socket.

U.S. Patent Provisional Application No. 62/007,742 of Geshlider et al. provides embodiments of a technology related to translating residual limb digital data (such as data that captures any of size, shape, and/or tissue density) into custom-shaped struts for assembly into a modular prosthetic socket. That application described a method that includes the following steps: acquiring digital data that characterizes the residual limb; packaging the digital limb data for downstream use; orienting the digital data onto a prosthetic socket structure; and applying the digital data to a thermal reforming of a stock strut to render a reformed strut, the reformed strut being complementary to a portion of the residual limb. In some embodiments, the acquired digital data provide a profile of an external physical boundary of the residual limb. In some embodiments, the acquired digital data may provide a profile of compliance of tissue underlying an external physical boundary of the residual limb.

U.S. Provisional Patent Application No. 62/007,742 describes approaches of acquiring digital data that are informative of residual limb physical aspects. As related therein, acquiring such data may occur by any suitable approach, including any one or more of scanning, photographing, casting, mapping with a three-dimensional point reference device a three-dimensional digital or physical representation of the residual limb, or by manual measurement. It is advantageous to map the residual limb to have digital data informative of the overall shape of the residual limb at a surface level. U.S. Provisional Pat. App. No. 62/007,742 further describes, in particular detail, an approach to acquiring digital data informative of the tissue underlying the surface, data that can differentiate among hard tissue, such as bone, dense tissue such as muscle, and soft tissue such as fat. Accordingly, in one embodiment, a method is directed acquiring spatial and compliance date from a fitting sock having one or more populations of sensors.

In a still further approach to acquiring digital data that characterizes a residual limb, photogrammetry may be applied. Photogrammetry is the practice of making measurements from photographs, especially for recovering the exact positions of surface points on a target object. The application of photogrammetry to deriving a useful 3D data of a target object such as a residual limb, as applied to creating an individualized, custom-built prosthetic socket, includes the use of multiple conventional 2D photographs of a residual limb. It is further anticipated that video data may be applicable to the method. The photogrammetric process is optimized by using a sufficiently large number of photographs, having the photographs taken from a sufficiently dense distribution of 3D perspectives, having a sufficiently dense set of unique markers on the target object, and/or having a sufficiently dense number of unique markers in the background.

Accordingly, per embodiments of a photogrammetric method to be applied to acquiring a 3D digital representation of a residual limb, an elastic sock is worn over the residual limb during a photographic session, the elastic sock having a surface with a high density of unique visual features. The sock is sufficiently elastic that it closely conforms, compliantly, to the residual limb in its natural form, but not sufficiently compressive that it significantly alters the natural form of the residual limb.

Further, per embodiments of a photogrammetric method, a sufficient number of photographs are taken of the residual limb. Further, such photographs are taken in a systematic pattern that converges on the residual limb from a substantially spherical perspective. Further, still, the photographs from the spherical perspective are taken at regular angular intervals.

Any suitable software application may be used to render 2D photographs into a 3D model. Currently available examples of appropriate software include Autodesk's ImageModeler™ or 123D Catch.

The present disclosure now expands on particular aspects of an approach to capturing patient-specific scan data and from such data, ultimately, generating strut surfaces for custom reforming of thermoplastic-fiber composite struts on a CNC controlled actuator.

A typical 3D format makes use of either a “point cloud” of X, Y, and Z coordinates in 3D space, a mesh connecting those coordinates, or any of the various file formats such as an STL, OBJ, PLY, VRML, etc., is used to contain this 3D information. In the example described here, scan data are from a Sense 3D Scanner unit (3D Systems, Inc.).

Scans of the residual limb can be taken in numerous different ways such as: (a) directly scanning the residual limb of a live patient, (b) scanning a positive plaster of Paris or solid material cast of a residual limb, (as traditionally used in the prosthetic socket industry), or (c) scanning a negative cast of a residual limb such as those commonly used in prosthetics practice. Scans can be performed on a reference surface with axial markings to provide an accurate XY alignment. Scans can also utilize various reference objects to further contribute to properly aligning the scan. One example would be to use a dowel positioned on the end of the residual limb.

The raw 3D scan file (for example, an STL file) is brought into a software application designed to manipulate such data, for the purposes of alignment and trimming. Some examples of this software are the Rhinoceros application (Robert McNeel & Associates) and Meshmixer (Autodesk Corporation).

One step in the process requires digitally moving and organizing parts of the modular prosthetic socket device with respect to the specific patient anatomical data. This can be done in various 3D CAD programs such Solidworks or Autodesk Inventor. Thus, one goal during the process is to prepare the 3D scan data for use in these programs. Conversion to surface formats such as IGS files satisfies this goal.

Another way to bring 3D scan data into programs such as Solidworks is to trim the scan to just a few key areas. The key areas of the scan data can be saved to use as a guide in the Solidworks application to create an abridged STL file, a subset of the larger STL file. (Solidworks cannot handle large scan data, thus, only a small amount of the file is saved.)

Further trimming and file manipulation is done to prepare the STL file for creating surface data, such as that available in Rhinoceros's Resurf Plug-In RhinoResurf is then used to create 2 surface files one of the upper portion and one of the lower portion. These are in IGS format. The Rhino file at this point contains both the mesh and the surface data within it. The two surface files (in IGS format) are brought into Solidworks and then joined into a single Solidworks part file.

The single Solidworks part is brought into an assembly in Solidworks that includes some of the parts of the modular prosthetic socket device as well as the abridged guide mesh discussed above. In this assembly, the hardware parts are moved around until in the correct place. Then four surfaces are extracted by saving the assembly as a Solidworks part file, and then by using various surfacing tools in Solidworks. These surfaces will become the physical struts to contribute to the formation of a modular prosthetic socket assembly. If desired, the four strut files can then be placed back into the original assembly to see how the device will look with the four struts in place.

The four strut files are saved as IGS surface files for use in CAM software such as InventorCAM (Autodesk, Inc). CAM software translates the surface data into machine language suitable for CNC machines such as that used for forming thermoplastic struts. The CAM software outputs text files for importing into the CNC machine.

### E. Custom Fitting of an Adjustable Ischial Seat

Custom-fitting or individual fitting of an ischial seat component of a prosthetic socket, as described herein, may occur by various approaches. In one approach, a height adjustable (or adjustable height) mechanism such as an ischial strut cap assembly 300 with a strut cap base 310 and an ischial seat pad 340 (FIGS. 7A-7O) allows a patient to control the height or elevation of an ischial seat above a distal base of a prosthetic socket. Such an adjustability mechanism is easily manipulated by a patient or a prosthetist, and the variation in height afforded by the mechanism is a high-resolution adjustment, as described in detail above. The height to which an ischial seat pad is elevated above the base of a socket is an important aspect the fit of a socket; such fit having a bearing on how effectively body weight load is preferentially directed through the patient's ischium, rather than through the distal end of the patient's residual limb. In addition to elevational adjustability, embodiments of the ischial seat pad 340 may also be laterally adjustable with respect to embodiments of strut cap base 310 (FIGS. 7R-7S), per embodiments of the invention.

In another approach, the particular specifications of ischial seat pad 340 may be custom-fitted to an individual patient. In contrast to the elevation or height of the ischial seat factor noted above, this particular aspect of custom fitting of an ischial seat relates to prosthetic socket effectively engaging the ischium conformationally, and taking full advantage of the dimensional and contouring aspects of the ischial tuberosity and the immediately surrounding muscular and skeletal features of that region of the pelvis. Custom fitting may also include any of adjusting the durometer, modulus of elasticity, or fill density of an ischial seat pad all of which may be controlled by parameters of a 3D printing process, per embodiments of the invention. FIGS. 8R-8Z, as noted above, show various ischial seat pad embodiments that have internal regions that vary in durometer. Further examples are described below in the context of FIGS. 15J-15L.

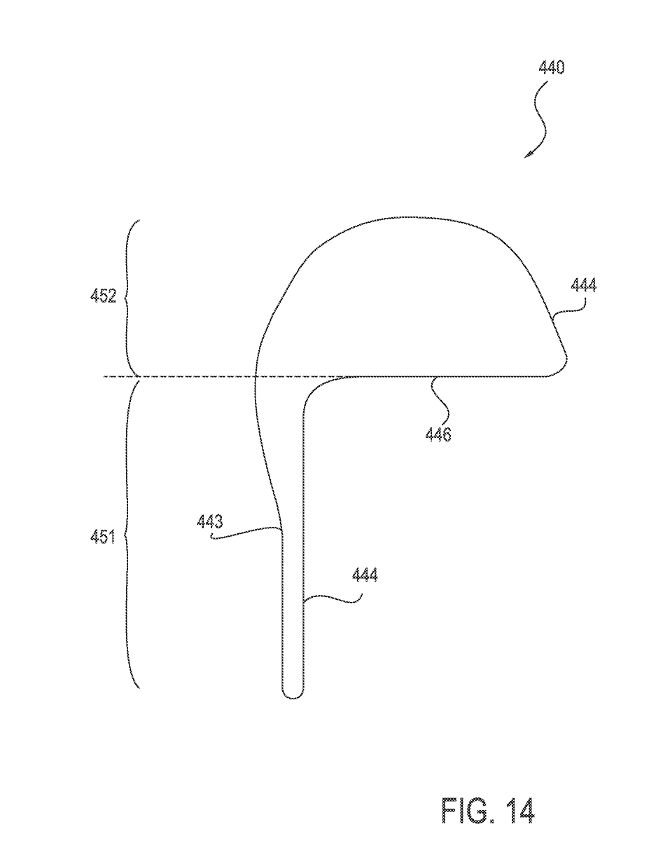

Custom fitting of devices or device components, in general, may be arrived at by more than one approach. In one approach, an entire device or component is fully custom-made (made specifically for an individual patient, based on a digital profile of the relevant body portion). In another approach, custom fitting is enabled by drawing components from an inventory of components that incorporate sufficient variable factors to multiplicatively provide a highly diverse set of size and shape options. Embodiments of a CAD model of an ischial seat that can be used as a driver in a 3D printing process, as described below, typically align with this second approach. However, a digital profile of the pelvic region of a patient centered on the ischium, may be also used to control the parametric variables associated with model shaping steps such that a model effectively becomes fully custom made. Aspects of embodiments of the invention that relate to providing a highly diverse inventory of ischial seat pads by way of a CAD model that can be shaped by a multitude of shaping steps are described further below in the context of FIGS. 14-18.

### Ischial Seat Pad Model Shaping and 3D Printing of Models to Produce Ischial Seat Embodiments

FIG. 14 shows a central cross sectional view of an embodiment of a CAD model of an ischial seat model 440 that has a constant portion 451 and a variable portion 452; embodiments of this model may be used to drive the 3D printing of ischial seat embodiments as seen in FIGS. 7A-8Z. CAD ischial seat model 440 is understandably very similar in appearance to an actual ischial seat 340 (as seen, for example, in FIG. 8A). With reference to FIG. 14, a constant portion 451 of CAD model 440 disposed below or distal to the distal horizontal surface 446 is substantially constant, constrained, or conserved throughout the various shaping steps (FIGS. 15B-15I) as this portion is configured to engage strut cap base 310 (FIGS. 7A-7O). Constant portion 451 is peninsula shaped, having an in internal surface 443 (facing internally into a prosthetic socket on which the adjustable height ischial strut cap assembly and seat pad is mounted) and an external surface (facing externally from the prosthetic socket) or strut-aligning surface 444. Constant portion 451 is complementary to a mounting element such as strut cap base 310, which serves to support ischial seat pad 340 on a prosthetic socket in such a location that seat pad 340 is positioned proximate the ischial tuberosity of the patient wearing the socket. It is the need to be complementary to such a mounting element that enforces the need for the constant portion 451 of CAD model 440 to be constant.

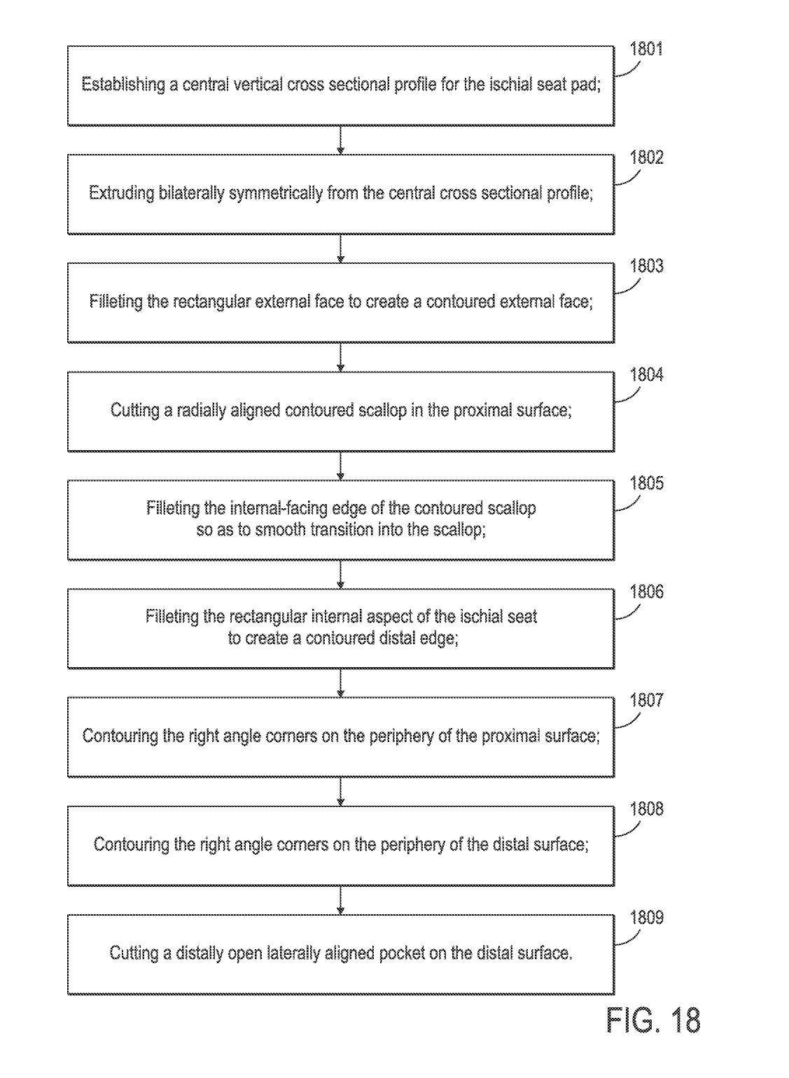

In contrast, a variable portion 452 of the model is disposed above the distal horizontal surface 446, such variability arising as a consequence of the parameters of the shaping steps described further below. The angles depicted in FIG. 15A (using FIG. 8A as a detailed reference): the 25° angle of projected vertex of the distal surface and the ischium contacting face, and the 90° angle of the intersection of the ischium contacting face and the external face) are merely examples of such angles, and may be varied within a collection of models, but remain constant as any single model progresses (FIGS. 15B-15I) through various shaping steps (FIG. 18).

Variable portion 452 of CAD model 440 includes an upper or proximal ischium contacting face 442, and external face 444, and a horizontal distal surface 446. The constant portion 451 of CAD model 440 includes an internal surface 443 and a strut aligning surface 447. The terms internal and external, as used herein, refer to the orientation of an ischial seat as it would be oriented when positioned atop a medial strut of an assembled socket (FIGS. 7N, 7O, 8A, 9D, and 9E). Per embodiments of the invention, CAD model 440 fully integrates constant portion 451 and variable portion 452.

FIGS. 15A-15I show a CAD model of an ischial seat pad 440 at various stages in its shaping. FIG. 15A shows an initial step of establishing a founding central vertical cross sectional profile for an ischial seat model 440A. This is a side view of that central vertical cross sectional profile.

FIG. 15B shows ischial seat model 440B, a product of a step of extruding bilaterally from the central cross sectional profile of the ischial seat model being prepared. This is a top perspective view of an internal aspect of the model. Variable parameters associated with the step include the absolute distance of extrusion on either side of the central cross sectional profile. Such extrusions can either be symmetrical or asymmetrical.

In FIGS. 15C-15I, dotted lines indicate the location of a cutting or filleting step that has occurred, and striped surfaces indicate surfaces newly exposed or created by the cutting or filleting step. FIG. 15C shows ischial seat model 440C, a product of a step of filleting the rectangular external face of the ischial seat model being prepared to create a contoured external face. This is a top perspective view of an internal aspect of the model. A variable parameter associated with this filleting step includes the portion of an arc encompassed by the fillet.

FIG. 15D shows ischial seat model 440D, a product of Va step of cutting a radially aligned scallop in the proximal surface of the ischial seat model being prepared. This is a top perspective view of an internal aspect of the model. A variable parameter associated with this cutting step includes the depth of the cut.

FIG. 15E shows ischial seat model 440E, a product of a step of filleting the internal-facing edge of the contoured scallop so as to smooth transition into the radially aligned scallop. This is a top perspective view of an internal aspect of the model. A variable parameter associated with this filleting step includes the distance into the fillet of FIG. 15D which is scalloped away.

FIG. 15F shows ischial seat model 440F, a product of a step of filleting a distal internal rectangular aspect of the ischial seat to create a contoured distal edge. This is a top perspective view of an internal aspect of the model. A variable parameter associated with this filleting step includes the portion of an arc encompassed by the fillet.

FIG. 15G shows ischial seat model 440G, a product of a step of contouring the right angle corners on the periphery of the proximal surface of the ischial seat being prepared. This is a top perspective view of an internal aspect of the model. A variable parameter associated with this contouring step includes the depth to which the right angle is shaved away.

FIG. 15H shows ischial seat model 440H, a product of a step of contouring the right angle corners on the periphery of the distal surface of the ischial seat being prepared. This is a bottom perspective view of the model. A variable parameter associated with this contouring step includes the depth to which the right angle is shaved away.

FIG. 15I shows a completed ischial seat model 440, a product of a step of cutting a distally open laterally aligned pocket on the distal surface of the ischial seat being prepared. This is a bottom perspective view of the model. Although the configuration of this cut may vary, it is substantially conserved because it is configured to engage (see FIGS. 7I-7M) positioning receptacle or features 347 on the proximal surface 325 of ischial seat pad 340.

FIGS. 15J-15L show examples of various applications of 3D printing technology to control internal distribution of regions of varying durometer by way of variable fill or variable density of 3D printed thermoplastic media. These embodiments are described in further detail below, in the context of such aspects of 3D printing and advantageous application of optional approaches to creating regions of variable density.

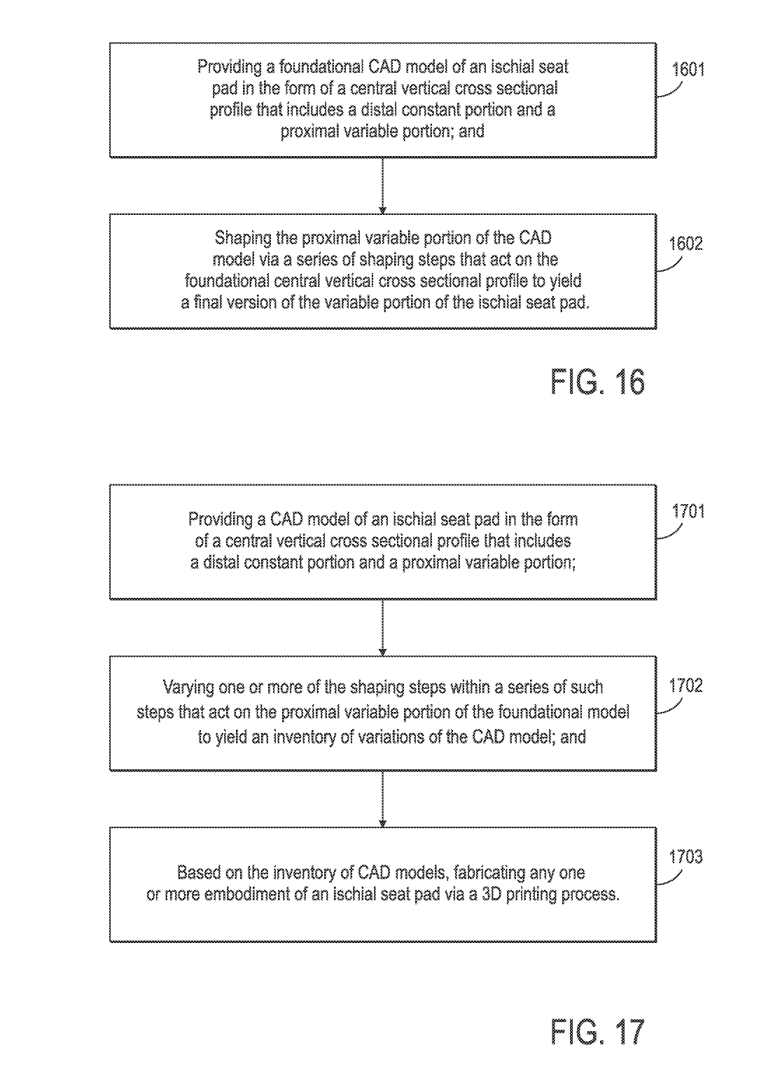

FIG. 16 is a flow diagram of an embodiment of a method of creating a CAD model of an ischial seat pad suitable for driving a 3D printing process to print embodiments of an ischial seat 340.
Step 1601 Providing a foundational CAD model of an ischial seat pad in the form of a central vertical cross sectional profile that includes a distal constant portion and a proximal variable portion; and
Step 1602 Shaping the proximal variable portion of the CAD model via a series of shaping steps that act on the foundational central vertical cross sectional profile to yield a final version of the variable portion of the ischial seat pad.

FIG. 17 is a flow diagram of an embodiment of a method of creating an inventory of CAD models of an ischial seat pad 440 that vary in form that are suitable for 3D printing of an inventory of ischial seat pads 340.
Step 1701 Providing a CAD model of an ischial seat pad in the form of a central vertical cross sectional profile that includes a distal constant portion and a proximal variable portion; and
Step 1702 Varying one or more shaping steps within a series of shaping steps that act on the proximal variable portion of the foundational model to yield an inventory of variations of the CAD model.
Step 1703 Based on the inventory of CAD models, fabricating any one or more embodiments of an ischial seat pad via a 3D printing process.

Embodiments of the shaping steps for the variable portion of the seat pad models in both FIGS. 16 and 17 may include steps such as those depicted in FIG. 18, and described below.

FIG. 18 is a flow diagram of an embodiment of a method of preparing an integral model of an ischial seat pad suitable for driving a 3D printing process. Steps of this method embodiment may include the following:
Step 1801 Establishing or selecting from an inventory a suitable baseline central vertical cross sectional profile for the ischial seat;
Step 1802 Extruding bilaterally symmetrically from the central cross sectional profile;
Step 1803 Filleting the rectangular external face to create a contoured external face;
Step 1804 Cutting a radially aligned contoured scallop in the proximal surface;
Step 1805 Filleting the internal-facing edge of the contoured scallop so as to smooth transition into the scallop;
Step 1806 Filleting the rectangular internal aspect of the ischial seat to create a contoured distal edge;
Step 1807 Contouring the right angle corners on the periphery of the proximal surface;
Step 1808 Contouring the right angle corners on the periphery of the distal surface; and
Step 1809 Cutting a distally open laterally aligned pocket on the distal surface.

With regard to the method steps recited above and depicted in FIG. 18, these are merely examples of steps, in an exemplary order. Other steps that start with a foundational model and result in or contribute to a finished ischial seat model embodiment are included in the scope of the invention. Not all steps listed above may be necessary, and the order of steps, in some instances, can be varied. Merely by way example, Step 1801 typically precedes all further steps, and Step 1802 needs to precede Step 1805, Step 1804 needs to precede step 1805. However, and by way of example, Step 1806 could precede Step 1803.

Each of the method steps, per this described embodiment, include a quantitative aspect, as for example, the depth of a cut or a filet in either absolute or relative terms, or an angle. Further, at least some aspect of each step is centered around the central vertical cross sectional profile established in step 1801. Typically, the action exercised in each step is symmetric across that central vertical cross sectional profile, but, alternatively any step may be asymmetric.

By varying quantitative details of the various steps shown in FIG. 18, such as by varying the angle of a filleting step or a contouring step, or varying the depth of a cut, or varying the shape or angle of the distal contoured surface of a founding vertical cross sectional profile, a large matrixed inventory of ischial seats can be created. FIGS. 8A-8Z depict an example of an inventory of ischial seats that vary in shape and/or dimensionality.

An inventory can take on one or more forms. In some embodiments, an inventory of this type can be built out as physical products and held in a conventional warehouse inventory. In some embodiments, a “virtual” inventory can be represented in a catalogue. In the instance of a “virtual” inventory, any particular ischial seat may be manufactured on demand by 3D printing methods, as described herein. Given the common aspects of manufacturing ischial seats to be held in a physical inventory and manufacturing an ischial seat on demand, the manufacturing on demand approach is practical, and likely to be cost effective.

The various model shaping steps, as shown in FIG. 18, can each be seen as multiplying factors that together create a standard inventory with a very large range of optional sizes and shapes of ischial seats. Inventors expect that such a large range of sizes and shapes will permit a fully satisfactory fitting for a substantial majority of patients. However, there also may be a population of patients who cannot be fitted by such a range of options. In those instances, per embodiments of the invention, by varying quantitative (e.g., dimensions, angles, depth) aspects of shaping method steps here described, if desired, a custom-fitted ischial seat may be created based on an acquired digital profile of a patient's ischium and surrounding pelvic bone structure. Any of the parameters of manufacturing a “standard” inventory of components can be varied so as to improve the fit of an ischial seat for a patient for whom a device drawn from the standard inventory is not satisfactory.

3D Printing of Ischial Seat Embodiments with a Variable Modulus of Elasticity

Returning now to an elaboration of using 3D printing technology and the fabrication of articles, such as an ischial seat pad 340 that have internal regions that vary in durometer in order to provide specific advantages related to comfort and therapeutic effectiveness, and which can be exploited for patient-specific customization purposes, as described above in the context of FIGS. 8R-8Z.

Embodiments of ischial seat pad 340 include articles that have regions that vary in durometer. FIGS. 15J-15L show cross sectional cutaway views of ischial seat embodiments that illustrate various approaches, per embodiments of the invention, to creating regions of variable fill density of 3D printed thermoplastic. One approach is to vary the relative presence of void volumes, as shown by ischial seat pad 340J in FIG. 15J. In this embodiment, spheroidal voids 340V within the printed fill are larger in the proximal region of ischial seat pad 340J, and gradually become smaller in the distal region. This distribution creates a relatively low durometer proximal region that absorbs force easily by way of compression, but as an impinging force compresses the seat further, the more thermoplastic-dense distal regions provide greater force resistance that prevents a bottoming out of the pad. Another approach, as shown by ischial seat pad 340K (FIG. 15K) where rather than a graduated change in durometer (as embodiment 15J of FIG. 15J), there are sharp demarcations into regions of different durometer such that a region a low durometer region 350 is disposed proximally high durometer region 352 is disposed distally and (as also shown earlier in FIG. 8W, for example). Ischial seat pad embodiment 340L of FIG. 15L shows yet another variation of variable thermoplastic media fill density capabilities. In this embodiment, an interior region 340I of relatively low durometer is overlaid by a “skin” 340S of higher fill density, and accordingly a high durometer.

As noted above, this application claims priority (among others) to U.S. Provisional Patent Application Ser. No. 62/163,717, filed May 19, 2015, entitled “Prosthetic socket distal cup with a strap lanyard suspension mechanism and a variable elastic modulus cushion”, which describes the 3D printing of therapeutic pads and cushions with a variable modulus of elasticity in detail, and which is incorporated herein. The terms “elastic” and “durometer” describe similar properties of material; an article that has a high modulus of elasticity (Young's modulus “E”) has a high resistance to being non-permanently deformed. An article having a high durometer has a high degree of hardness. Thus, generally, an article having a high modulus of elasticity also has a high durometer. Both terms (elasticity and durometer) are applicable to material properties that are associated with fill density. Modulus of elasticity includes directionality features. Durometer generally refers to hardness of an object, as a whole, an article may have moduli of elasticity that vary according to the direction of the deforming force applied to it. Accordingly, this property of directionality can be controlled through the design delivered by way of 3D printing, and such controllable directionality of varying elasticity (or hardness) can be exploited for therapeutic advantage.

Variable elastic modulus pads or cushions have widespread applicability to devices that interface with the body, particularly at sites where a graded quality of padding is appropriate or preferable, or when the site being padded is one where the interfacing body part is sensitive or vulnerable, and/or where the site is one through which the transfer of forces relates to functionality of the padding article. It is a challenge for a pad or cushion to elastically engage at low levels of impinging force and not “bottom out” at a high level of impinging force. If a pad bottoms out, then it actually no longer is acting as shock absorbing pad. And, on the other hand, if a pad is too hard (albeit not bottoming out), it also does not really fulfill its mission or potential as a shock absorbing pad. Thus, embodiments of a variable elastic modulus ischial seat pad 340, as provided herein, typically have a low elastic modulus on a surface that engages the body, and the elastic modulus increases with increasing depth within the pad. The effect of such embodiments is that they provide a graded range of elasticity or hardness, and one that can be controlled by the design of the pattern and density of the 3D printed thermoplastic fill.

The embodiments of a variable elastic modulus ischial seat pad 340 provided herein represent but one of many examples of the utility of such pad. As a non-comprehensive listing, other examples include sites in prosthetic devices other than the distal cup of a prosthetic socket, orthotic devices, exoskeletal devices, gripping elements in tools or utensils, and sites on walkers, canes, wheelchairs, stationary chairs, and beds. Typically, variable elastic modulus ischial seat pad embodiments 340 are formed by 3D printing process. Accordingly, such pad can also assume custom-shaping aspects or surface that conform to a body portion. Aspects of custom shaping prosthetic socket components by way of mass customization methods of manufacturing are described in U.S. patent application Ser. No. 14/572,571, as filed on Dec. 16, 2014, and U.S. Provisional Patent Application No. 62/007,742, as filed on Jun. 4, 2014. 3D printing methods offer a number of advantages over conventional approaches to manufacturing a variable elastic modulus pad or similar product that might approximate these structures, included among the advantages is the absence of requiring molds, which themselves, consume resource.

### Alternative Embodiments of an Adjustable Height Ischial Seat

FIG. 19A shows a top perspective view of a circumferentially configured laminated prosthetic socket 500 with a height adjustable ischial seat assembly 501 disposed on its medial aspect at an adjustable height ischial seat assembly mounting site 502 and a distal base 503. FIG. 19B shows a side face view of the circumferentially configured laminated prosthetic socket frame 500 with a height adjustable ischial seat 501 disposed on its medial aspect. Mechanical aspects of height adjustable seat assembly 501 substantially as described above in context of FIGS. 7A-7O.

Although in this patent application embodiments of an adjustable height ischial seat assembly 300 are described and shown (FIG. 4B) generally in the context of a modular prosthetic socket 10, and more particularly disposed at the proximal end of a medial strut 20M, embodiments of an adjustable height ischial seat may be disposed in other and, indeed, in most types of prosthetic socket. In this particular example (FIGS. 19A-19B), an adjustable height ischial seat assembly 501 may be appropriately disposed with a circumferentially configured laminated plastic prosthetic socket frame 500. Adjustable height ischial seat assembly 501 is disposed on socket frame 500 at a location (as determined by the overall geometry of socket frame 500) where a proximal aspect of ischial seat assembly 501 will engage the ischium of a patient wearing the socket. By means of adjusting a height adjustable mechanism of ischial seat assembly 501, the distance between (or elevation of) the proximal aspect of ischial seat and distal base 503 can be adjusted and personally optimized by a patient, as described elsewhere herein, in context of describing embodiments depicted in FIGS. 7A-7O.

Although this invention has been disclosed in the context of certain embodiments and examples, the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. A modular prosthetic socket for a residual limb of a lower extremity of a patient, the socket comprising:
a base plate, comprising multiple peripherally disposed strut connecting sites;
multiple longitudinal struts, each strut comprising a thermoplastic-fiber composite material, a proximal end and a distal end, wherein the distal end of each strut is connected to the base plate at one of the peripherally disposed strut connecting sites, and wherein one of the struts is a medial strut, configured to occupy a medial position with respect to the residual limb when the prosthetic socket is worn by the patient; and
an ischial seat pad adjustably coupled with the proximal end of the medial strut, such that the ischial seat pad is vertically adjustable relative to the medial strut to adjust a total length of the medial strut measured from a proximal end of the ischial seat pad to the distal end of the medial strut, wherein the ischial seat pad is configured to engage an ischium of the patient when the prosthetic socket is worn by the patient.

2. The modular prosthetic socket of claim 1, wherein the ischial seat pad comprises one component of an adjustable-height medial strut cap assembly of the socket, and wherein the strut cap assembly further includes a strut cap base attached to the ischial seat pad for adjustably coupling the ischial seat pad with the medial strut.

3. The modular prosthetic socket of claim 2, wherein the strut cap base is drawn from a collection of strut cap bases that includes multiple different sizes and shapes of strut cap bases configured to fit over multiple sizes and shapes of medial struts.

4. The modular prosthetic socket of claim 1, wherein the ischial seat pad is drawn from a collection of ischial seat pads that includes multiple different sizes and shapes of ischial seat pads.

5. The modular prosthetic socket of claim 1, further comprising a strut cap base for coupling the ischial seat pad with the medial strut, the strut cap base comprising an adjustable mechanism that allows a vertical position of the strut cap base relative to the medial strut to be adjusted and locked.

6. The modular prosthetic socket of claim 5, wherein the ischial seat pad is horizontally slidable with respect to the strut cap base.

7. The prosthetic socket of claim 5, wherein the strut cap base fits over the proximal end of the medial strut, wherein the strut cap base comprises an internal face, an external face, a proximal end, and a distal end comprising an opening to accommodate the proximal end of the medial strut, and wherein the ischial seat pad fits over the proximal end of the strut cap base and comprises a contoured proximal ischium-contacting face, a distally extending internal portion, and a distal surface configured to align against the proximal end of the strut cap base.

8. The modular prosthetic socket of claim 1, further comprising a sensor in the ischial seat pad for sensing an amount of force exerted on the ischial seat pad by the patient during use of the prosthetic socket.

9. A height adjustable strut cap assembly for a prosthetic socket for a residual limb of a lower extremity of a patient, the strut cap assembly comprising:
a strut cap base comprising an adjustable, lockable attachment mechanism for attaching to a medial strut of the prosthetic socket such that the strut cap base is vertically adjustable relative to the medial strut and is configured to be locked in position at a desired position relative to the medial strut; and
an ischial seat pad comprising a distal surface configured to be securable to a proximal aspect of the strut cap base and a proximal surface configured to engage an ischium of a patient wearing the prosthetic socket.

10. The strut cap assembly of claim 9, wherein the strut cap assembly comprises an open distal end sized and configured to fit over a proximal end of the medial strut.

11. The strut cap assembly of claim 9, wherein the adjustment mechanism comprises a friction-based arrangement, such that the strut cap base can be releasably pressed against the medial strut, wherein in the absence of friction the strut cap base can move vertically with respect the medial strut, and wherein when friction is applied, the strut cap base is locked into a fixed vertical position on the medial strut.

12. The strut cap assembly of claim 9, further comprising a sensor in the ischial seat pad for sensing an amount of force exerted on the ischial seat pad by the patient during use of the prosthetic socket.

13. The strut cap assembly of claim 9, wherein the ischial seat pad comprises a contoured proximal ischium-contacting face, a distally extending internal portion sized and configured to align against an internal face of a proximal portion of the medial strut, and a distal surface configured to align against the proximal end of the strut cap base.

14. A method for generating a computer-aided design (CAD) model of an ischial seat pad for a prosthetic socket that is suitable for 3D printing, the method comprising:
generating a constant portion of the CAD model of the ischial seat pad, wherein the constant portion comprises a shape complementary to a mounting element configured to allow mounting of the ischial seat pad on the prosthetic socket; and
generating a variable portion of the CAD model of the ischial seat pad, wherein the variable portion is variable according to at least one of quantitative or geometric parameters within each of a series of shaping steps that are applied to a founding central vertical cross sectional profile for an ischial seat pad model, the central vertical cross sectional profile comprising a proximal surface with an angle of inclination.

15. The method of claim 12, wherein generating the variable portion comprises applying the series of shaping steps, and wherein the series of shaping steps comprises:
establishing the founding central vertical cross sectional profile for the ischial seat pad model;
extruding bilaterally symmetrically from the central cross sectional profile to create an in-progress 3D model of the ischial seat pad;
filleting a rectangular external face of the ischial seat pad model being prepared to create a contoured external face on the in-progress 3D model;
cutting a radially aligned scallop in the proximal surface of the in-progress 3D model;
filleting an internal-facing edge of the contoured scallop of the in-progress 3D model so as to smooth a transition into the radially aligned scallop;
filleting a distal internal rectangular aspect of the in-progress 3D model to create a contoured distal edge;
contouring right angle corners on a periphery of a proximal surface of the in-progress 3D model;
contouring right angle corners on a periphery of a distal surface of the in-progress 3D model; and
cutting a distally open laterally aligned pocket on the distal surface of the ischial seat pad to complete the 3D model of the ischial seat pad.

16. The method of claim 15, further comprising manufacturing the ischial seat pad by printing, using 3D printing technology, the ischial seat pad from the 3D model.

17. The method of claim 15, further comprising repeating the series of shaping steps while varying at least one of a dimensionality or an angulation of at least one of the series of shaping steps to provide a different 3D model for a different ischial seat pad.

18. The method of claim 17, further comprising manufacturing the ischial seat pad and the different ischial seat pad by printing, using 3D printing technology, the ischial seat pad and the different ischial seat pad from the 3D model and the different 3D model, respectively.

19. A method for manufacturing an ischial seat pad for a prosthetic socket, using an ischial seat pad model that is suitable for 3D printing, the method comprising:
generating a constant portion of the CAD model of the ischial seat pad, wherein the constant portion comprises a shape complementary to a mounting element configured to allow mounting of the ischial seat pad on the prosthetic socket;
generating a variable portion of the CAD model of the ischial seat pad, wherein the variable portion is variable according to at least one of quantitative or geometric parameters within each of a series of shaping steps that are applied to a founding central vertical cross sectional profile for an ischial seat pad model, the central vertical cross sectional profile comprising a proximal surface with an angle of inclination; and
using the CAD model to manufacture the ischial seat pad.

20. The method of claim 19, wherein using the CAD model comprises printing the ischial seat pad from the CAD model using a 3D printing technology.

21. The method of claim 19, further comprising, before generating the variable portion of the CAD model:
acquiring a 3D digital profile of a region of a patient's pelvis surrounding an ischial tuberosity in the form of an STL file; and
importing the STL file into a CAD application.
